(19) United States
(12) Patent Application Publication
(10) Pub. No.: US 2019/0055235 A1
(43) Pub. Date: Feb. 21, 2019

(54) SUBSTITUTED BICYCLIC COMPOUNDS AS BROMODOMAIN INHIBITORS

(71) Applicant: ZENITH EPIGENETICS LTD., Calgary (CA)

(72) Inventors: Samuel David Brown, San Carlos, CA (US); Craig Alan COBURN, San Rafael, CA (US)

(21) Appl. No.: 15/534,165

(22) PCT Filed: Dec. 16, 2015
(86) PCT No.: PCT/IB2015/002522

§ 371 Date: Jun. 8, 2017

Related U.S. Application Data
(60) Provisional application No. 62/093,386, filed on Dec. 17, 2014.

(51) Int. Cl. C07D 471/04; A61K 31/437; C07D 413/14; A61K 31/454; A61K 31/496; A61K 31/5377; A61K 45/06; A61P 35/00

(52) U.S. Cl. CPC C07D 471/04; A61K 31/437; C07D 413/14; A61P 35/00; A61K 31/496; A61K 31/5377; A61K 45/06; A61K 31/454

(57) ABSTRACT

The present disclosure relates to substituted bicyclic compounds, which are useful for inhibition of BET protein function by binding to bromodomains, and their use in therapy.

This application claims priority from U.S. Provisional Patent Application No. 62/093,386, filed Dec. 17, 2014, which is hereby incorporated by reference in its entirety.

The invention provides novel compounds, pharmaceutical compositions containing such compounds, and their use in prevention and treatment of diseases and conditions associated with bromodomain and extra terminal domain (BET) proteins.

Post-translational modifications (PTMs) of histones are involved in regulation of gene expression and chromatin organization in eukaryotic cells. Histone acetylation at specific lysine residues is a PTM that is regulated by histone acetylases (HATs) and deacetylases (HDACs). Peserico, A. and C. Simone, “Physical and functional HAT/HDAC interplay regulates protein acetylation balance,” J Biomed Biotechnol, 2011:371832 (2011). Small molecule inhibitors of HDACs and HATs are being investigated as cancer therapy. Hoshino, I. and H. Matsubara, “Recent advances in histone deacetylase targeted cancer therapy” Surg Today 40(9):809-15 (2010); Vernarecci, S., F. Tosi, and P. Filetici, “Tuning acetylated chromatin with HAT inhibitors: a novel tool for therapy” Epigenetics 5(2):105-11 (2010); Bandyopadhyay, K., et al., “Spermidinyl-CoA-based HAT inhibitors block DNA repair and provide cancer-specific chemo- and radiosensitization,” Cell Cycle 8(17):2779-88 (2009); Arif, M., et al., “Protein lysine acetylation in cellular function and its role in cancer manifestation,” Biochim Biophys Acta 1799(10-12):702-16 (2010). Histone acetylation controls gene expression by recruiting protein complexes that bind directly to acetylated lysine via bromodomains. Sanchez, R. and M. M. Zhou, “The role of human bromodomains in chromatin biology and gene transcription,” Curr Opin Drug Discov Devel 12(5):659-65 (2009). One such family, the bromodomain and extra terminal domain (BET) proteins, comprises Brd2, Brd3, Brd4, and BrdT, each of which contains two bromodomains in tandem that can independently bind to acetylated lysines, as reviewed in Wu, S. Y. and C. M. Chiang, “The double bromodomain-containing chromatin adaptor Brd4 and transcriptional regulation,” J Biol Chem 282(18):13141-5 (2007).

Interfering with BET protein interactions via bromodomain inhibition results in modulation of transcriptional programs that are often associated with diseases characterized by dysregulation of cell cycle control, inflammatory cytokine expression, viral transcription, hematopoietic differentiation, insulin transcription, and adipogenesis. Belkina, A. C. and G. V. Denis, “BET domain co-regulators in obesity, inflammation and cancer,” Nat Rev Cancer 12(7):465-77 (2012). BET inhibitors are believed to be useful in the treatment of diseases or conditions related to systemic or tissue inflammation, inflammatory responses to infection or hypoxia, cellular activation and proliferation, lipid metabolism, fibrosis, and the prevention and treatment of viral infections. Belkina, A. C. and G. V. Denis, “BET domain co-regulators in obesity, inflammation and cancer,” Nat Rev Cancer 12(7):465-77 (2012); Prinjha, R. K., J. Witherington, and K. Lee, “Place your BETs: the therapeutic potential of bromodomains,” Trends PharmacolSci 33(3):146-53 (2012).

Autoimmune diseases, which are often chronic and debilitating, are a result of a dysregulated immune response, which leads the body to attack its own cells, tissues, and organs. Pro-inflammatory cytokines including IL-1β, TNF-α, IL-6, MCP-1, and IL-17 are overexpressed in autoimmune disease. IL-17 expression defines the T cell subset known as Th17 cells, which are differentiated, in part, by IL-6, and drive many of the pathogenic consequences of autoimmune disease. Thus, the IL-6/Th17 axis represents an important, potentially druggable target in autoimmune disease therapy. Kimura, A. and T. Kishimoto, “IL-6: regulator of Treg/Th17 balance,” Eur J Immunol 40(7):1830-5 (2010). BET inhibitors are expected to have anti-inflammatory and immunomodulatory properties. Belkina, A. C. and G. V. Denis, “BET domain co-regulators in obesity, inflammation and cancer,” Nat Rev Cancer 12(7):465-77 (2012); Prinjha, R. K., J. Witherington, and K. Lee, “Place your BETs: the therapeutic potential of bromodomains,” Trends Pharmacol Sci 33(3):146-53 (2012). BET inhibitors have been shown to have a broad spectrum of anti-inflammatory effects in vitro including the ability to decrease expression of pro-inflammatory cytokines such as IL-1β, MCP-1, TNF-α, and IL-6 in activated immune cells. Mirguet, O., et al., “From ApoA1 upregulation to BET family bromodomain inhibition: discovery of I-BET151,” Bioorg Med Chem Lett 22(8):2963-7 (2012); Nicodeme, E., et al., “Suppression of inflammation by a synthetic histone mimic,” Nature 468(7327):1119-23 (2010); Seal, J., et al., “Identification of a novel series of BET family bromodomain inhibitors: binding mode and profile of I-BET151 (GSK1210151A),” Bioorg Med Chem Lett 22(8):2968-72 (2012). The mechanism for these anti-inflammatory effects may involve BET inhibitor disruption of Brd4 co-activation of NF-κB-regulated pro-inflammatory cytokines and/or displacement of BET proteins from cytokine promoters, including IL-6. Nicodeme, E., et al., “Suppression of inflammation by a synthetic histone mimic,” Nature 468(7327):1119-23 (2010); Zhang, G., et al., “Down-regulation of NF-kappaB Transcriptional Activity in HIVassociated Kidney Disease by BRD4 Inhibition,” J Biol Chem, 287(34):8840-51 (2012); Zhou, M., et al., “Bromodomain protein Brd4 regulates human immunodeficiency virus transcription through phosphorylation of CDK9 at threonine 29,” J Virol 83(2):1036-44 (2009). In addition, because Brd4 is involved in T-cell lineage differentiation, BET inhibitors may be useful in inflammatory disorders characterized by specific programs of T cell differentiation. Zhang, W. S., et al., “Bromodomain-Containing-Protein 4 (BRD4) Regulates RNA Polymerase II Serine 2 Phosphorylation in Human CD4+ T Cells,” J Biol Chem (2012).

The anti-inflammatory and immunomodulatory effects of BET inhibition have also been confirmed in vivo. A BET inhibitor prevented endotoxin- or bacterial sepsis-induced death and cecal ligation puncture-induced death in mice, suggesting utility for BET inhibitors in sepsis and acute inflammatory disorders. Nicodeme, E., et al., “Suppression of inflammation by a synthetic histone mimic,” Nature 468(7327):1119-23 (2010). A BET inhibitor has been shown to ameliorate inflammation and kidney injury in HIV-1 transgenic mice, an animal model for HIV-associated nephropathy, in part through inhibition of Brd4 interaction with NF-κB. Zhang, G., et al., “Down-regulation of NF-kappaB Transcriptional Activity in HIV associated Kidney Disease by BRD4 Inhibition,” J Biol Chem 287(34):8840-51 (2012). The utility of BET inhibition in autoimmune disease was demonstrated in a mouse model of multiple sclerosis, where BET inhibition resulted in abrogation of clinical signs of disease, in part, through inhibition of IL-6 and IL-17. R. Jahagirdar, S. M. et al., “An Orally Bioavailable Small Molecule RVX-297 Significantly Decreases Disease in a Mouse Model of Multiple Sclerosis,” World Congress of Inflammation, Paris, France (2011). These results were supported in a similar mouse model where it was shown that treatment with a BET inhibitor inhibited T cell differentiation into pro-autoimmune Th1 and Th17 subsets in vitro, and further abrogated disease induction by pro-inflammatory Th1 cells. Bandukwala, H. S., et al., “Selective inhibition of CD4+ T-cell cytokine production and autoimmunity by BET protein and c-Myc inhibitors,” Proc Natl Acad Sci USA 109(36):14532-7 (2012).

BET inhibitors may be useful in the treatment of a variety of chronic autoimmune inflammatory conditions. Thus, one aspect of the invention provides compounds, compositions, and methods for treating autoimmune and/or inflammatory diseases by administering one or more compounds of the invention or pharmaceutical compositions comprising one or more of those compounds. Examples of autoimmune and inflammatory diseases, disorders, and syndromes that may be treated using the compounds and methods of the invention include but are not limited to, inflammatory pelvic disease, urethritis, skin sunburn, sinusitis, pneumonitis, encephalitis, meningitis, myocarditis, nephritis (Zhang, G., et al., “Down-regulation of NF-kappaB Transcriptional Activity in HIVassociated Kidney Disease by BRD4 Inhibition,” J Biol Chem 287(34):8840-51 (2012)), osteomyelitis, myositis, hepatitis, gastritis, enteritis, dermatitis, gingivitis, appendicitis, pancreatitis, cholecystitis, agammaglobulinemia, psoriasis, allergy, Crohn's disease, irritable bowel syndrome, ulcerative colitis (Prinjha, R. K., J. Witherington, and K. Lee, “Place your BETs: the therapeutic potential of bromodomains,” Trends Pharmacol Sci 33(3):146-53 (2012)), Sjogren's disease, tissue graft rejection, hyperacute rejection of transplanted organs, asthma, allergic rhinitis, chronic obstructive pulmonary disease (COPD), autoimmune polyglandular disease (also known as autoimmune polyglandular syndrome), autoimmune alopecia, pernicious anemia, glomerulonephritis, dermatomyositis, multiple sclerosis (Bandukwala, H. S., et al., “Selective inhibition of CD4+ T-cell cytokine production and autoimmunity by BET protein and c-Myc inhibitors,” Proc Natl Acad Sci USA 109(36):14532-7 (2012)), scleroderma, vasculitis, autoimmune hemolytic and thrombocytopenic states, Goodpasture's syndrome, atherosclerosis, Addison's disease, Parkinson's disease, Alzheimer's disease, Type I diabetes (Belkina, A. C. and G. V. Denis, “BET domain co-regulators in obesity, inflammation and cancer,” Nat Rev Cancer 12(7):465-77 (2012)), septic shock (Zhang, G., et al., “Down-regulation of NF-kappaB Transcriptional Activity in HIVassociated Kidney Disease by BRD4 Inhibition,” J Biol Chem 287(34):8840-51 (2012)), systemic lupus erythematosus (SLE) (Prinjha, R. K., J. Witherington, and K. Lee, “Place your BETs: the therapeutic potential of bromodomains,” Trends Pharmacol Sci 33(3):146-53 (2012)), rheumatoid arthritis (Denis, G. V., “Bromodomain coactivators in cancer, obesity, type 2 diabetes, and inflammation,” Discov Med 10(55):489-99 (2010)), psoriatic arthritis, juvenile arthritis, osteoarthritis, chronic idiopathic thrombocytopenic purpura, Waldenstrom macroglobulinemia, myasthenia gravis, Hashimoto's thyroiditis, atopic dermatitis, degenerative joint disease, vitiligo, autoimmune hypopituitarism, Guillain-Barre syndrome, Behcet's disease, uveitis, dry eye disease, scleroderma, mycosis fungoides, and Graves' disease.

BET inhibitors may be useful in the treatment of a wide variety of acute inflammatory conditions. Thus, one aspect of the invention provides compounds, compositions, and methods for treating inflammatory conditions including but not limited to, acute gout, nephritis including lupus nephritis, vasculitis with organ involvement, such as, e.g., glomerulonephritis, vasculitis, including giant cell arteritis, Wegener's granulomatosis, polyarteritis nodosa, Behcet's disease, Kawasaki disease, and Takayasu's arteritis.

BET inhibitors may be useful in the prevention and treatment of diseases or conditions that involve inflammatory responses to infections with bacteria, viruses, fungi, parasites, and their toxins, such as, but not limited to sepsis, sepsis syndrome, septic shock (Nicodeme, E., et al., “Suppression of inflammation by a synthetic histone mimic,” Nature 468(7327):1119-23 (2010)), systemic inflammatory response syndrome (SIRS), multi-organ dysfunction syndrome, toxic shock syndrome, acute lung injury, adult respiratory distress syndrome (ARDS), acute renal failure, fulminant hepatitis, burns, post-surgical syndromes, sarcoidosis, Herxheimer reactions, encephalitis, myelitis, meningitis, malaria, and SIRS associated with viral infections, such as, e.g., influenza, herpes zoster, herpes simplex, and coronavirus. Belkina, A. C. and G. V. Denis, “BET domain co-regulators in obesity, inflammation and cancer,” Nat Rev Cancer 12(7):465-77 (2012). Thus, one aspect of the invention provides compounds, compositions, and methods for treating these inflammatory responses to infections with bacteria, viruses, fungi, parasites, and their toxins described herein.

Cancer is a group of diseases caused by dysregulated cell proliferation. Therapeutic approaches aim to decrease the numbers of cancer cells by inhibiting cell replication or by inducing cancer cell differentiation or death, but there is still significant unmet medical need for more efficacious therapeutic agents. Cancer cells accumulate genetic and epigenetic changes that alter cell growth and metabolism, promoting cell proliferation and increasing resistance to programmed cell death, or apoptosis. Some of these changes include inactivation of tumor suppressor genes, activation of oncogenes, and modifications of the regulation of chromatin structure, including deregulation of histone PTMs. Watson, J. D., “Curing ‘incurable’ cancer,” Cancer Discov 1(6):477-80 (2011); Morin, R. D., et al., “Frequent mutation of histone-modifying genes in non-Hodgkin lymphoma” Nature 476(7360):298-303 (2011).

One aspect of the invention provides compounds, compositions, and methods for treating human cancer, including, but not limited to, cancers that result from aberrant translocation or overexpression of BET proteins (e.g., NUT midline carcinoma (NMC) (French, C. A., “NUT midline carcinoma,” Cancer Genet Cytogenet 203(1):16-20 (2010) and B-cell lymphoma (Greenwald, R J., et al., “E mu-BRD2 transgenic mice develop B-cell lymphoma and leukemia,” Blood 103(4):1475-84 (2004)). NMC tumor cell growth is driven by a translocation of the Brd4 or Brd3 gene to the nutlin 1 gene. Filippakopoulos, P., et al., “Selective inhibition of BET bromodomains,” Nature 468(7327):1067-73 (2010). BET inhibition has demonstrated potent antitumor activity in murine xenograft models of NMC, a rare but lethal form of cancer. The present disclosure provides a method for treating human cancers, including, but not limited to, cancers dependent on a member of the myc family of oncoproteins including c-myc, MYCN, and L-myc. Vita, M. and M. Henriksson, “The Myc oncoprotein as a therapeutic target for human cancer,” Semin Cancer Biol 16(4):318-30 (2006). These cancers include Burkitt's lymphoma, acute myelogenous leukemia, multiple myeloma, and aggressive human medulloblastoma. Vita, M. and M. Henriksson, “The Myc oncoprotein as a therapeutic target for human cancer,” Semin Cancer Biol 16(4):318-30 (2006). Cancers in which c-myc is overexpressed may be particularly susceptible to BET protein inhibition; it has been shown that treatment of tumors that have activation of c-myc with a BET inhibitor resulted in tumor regression through inactivation of c-myc transcription. Dawson, M. A., et al., “Inhibition of BET recruitment to chromatin as an effective treatment for MLL-fusion leukaemia,” Nature 478(7370):529-33 (2011); Delmore, I. E., et al., “BET bromodomain inhibition as a therapeutic strategy to target c-Myc,” Cell 146(6):904-17 (2010); Mertz, J. A., et al., “Targeting MYC dependence in cancer by inhibiting BET bromodomains,” Proc Natl Acad Sci USA 108(40):16669-74 (2011); Ott, C J., et al., “BET bromodomain inhibition targets both c-Myc and IL7R in highrisk acute lymphoblastic leukemia,” Blood 120(14):2843-52 (2012); Zuber, J., et al., “RNAi screen identifies Brd4 as a therapeutic target in acute myeloid leukaemia,” Nature 478(7370):524-8 (2011).

Embodiments of the invention include methods for treating human cancers that rely on BET proteins and pTEFb (Cdk9/CyclinT) to regulate oncogenes (Wang, S. and P. M. Fischer, “Cyclin-dependent kinase 9: a key transcriptional regulator and potential drug target in oncology, virology and cardiology,” Trends Pharmacol Sci 29(6):302-13 (2008)), and cancers that can be treated by inducing apoptosis or senescence by inhibiting Bcl2, cyclin-dependent kinase 6 (CDK6) (Dawson, M. A., et al., “Inhibition of BET recruitment to chromatin as an effective treatment for MLL-fusion leukaemia,” Nature 478(7370):529-33 (2011)), or human telomerase reverse transcriptase (hTERT) (Delmore, J. E., et al., “BET bromodomain inhibition as a therapeutic strategy to target c-Myc,” Cell 146(6):904-17 (2010); Ruden, M. and N. Purl, “Novel anticancer therapeutics targeting telomerase,” Cancer Treat Rev 39(5):444-456 (2012)).

Inhibition of BET proteins may also result in inhibition of enhancer and/or super-enhancer known to drive transcriptional programs associated with several human disease etiologies (Hnisz, D. et al. “Super-enhancers in the control of cell identity and disease,” Cell 155:934-947 (2013); Loven, J. et al. “Selective inhibition of tumor oncogenes by disruption of super-enhancers.” Cell 153: 320-334 (2013); Whyte, W. A. et al. “Master transcription factors and mediator establish super-enhancers at key cell identity genes,” Cell 153:307-319 (2013)). The MYC oncogene is an example of a gene associated with a super enhancer that is disrupted by BET-bromodomain inhibitors. See, e.g., Loven (2013). Thus, one aspect of the invention provides compounds, compositions, and methods for treating such diseases and disorders, including cancers associated with a super-enhancer or enhancer that may be disrupted with a BET inhibitor.

BET inhibitors may be useful in the treatment of cancers including, but not limited to, adrenal cancer, acinic cell carcinoma, acoustic neuroma, acral lentiginous melanoma, acrospiroma, acute eosinophilic leukemia, acute erythroid leukemia, acute lymphoblastic leukemia (see, e.g., Loven (2013)), acute megakaryoblastic leukemia, acute monocytic leukemia, acute myeloid leukemia (Dawson, M A., et al., “Inhibition of BET recruitment to chromatin as an effective treatment for MLL-fusion leukaemia,” Nature 478(7370):529-33 (2011); Mertz, J. A., et al., “Targeting MYC dependence in cancer by inhibiting BET bromodomains,” Proc Natl Acad Sci USA 108(40):16669-74 (2011); Zuber, J., et al., “RNAi screen identifies Brd4 as a therapeutic target in acute myeloid leukaemia,” Nature 478(7370):524-8 (2011)), adenocarcinoma, adenoid cystic carcinoma, adenoma, adenomatoid odontogenic tumor, adenosquamous carcinoma, adipose tissue neoplasm, adrenocortical carcinoma, adult T-cell leukemia/lymphoma (Wu, X. et al. “Bromodomain and extraterminal (BET) protein inhibition suppresses human T cell leukemia virus 1 (HTLV-1) Tax protein-mediated tumorigenesis by inhibiting nuclear factor kappaB (NF-kappaB) signaling,” J Biol Chem 288:36094-36105 (2013)), aggressive NK-cell leukemia, AIDS-related lymphoma, alveolar rhabdomyosarcoma, alveolar soft part sarcoma, ameloblastic fibroma, anaplastic large cell lymphoma, anaplastic thyroid cancer, angioimmunoblastic T-cell lymphoma (Knoechel, B. et al. “An epigenetic mechanism of resistance to targeted therapy in T cell acute lymphoblastic leukemia,” Not Genet 46:364-370 (2014); Loosveld, M. et al. “Therapeutic Targeting of c-Myc in T-Cell Acute Lymphoblastic Leukemia (T-ALL),” Oncotarget 5(10):3168-72 (2014); Reynolds, C. et al. “Repression of BIM mediates survival signaling by MYC and AKT in high-risk T-cell acute lymphoblastic leukemia,” Leukemia 28(9):1819-27 (2014); Roderick, J. E. et al. “c-Myc inhibition prevents leukemia initiation in mice and impairs the growth of relapsed and induction failure pediatric T-ALL cells,” Blood 123:1040-1050 (2014)), angiomyolipoma, angiosarcoma, astrocytoma, atypical teratoid rhabdoid tumor, B-cell acute lymphoblastic leukemia (Ott, C J., et al., “BET bromodomain inhibition targets both c-Myc and IL7R in highrisk acute lymphoblastic leukemia,” Blood 120(14):2843-52 (2012)), B-cell chronic lymphocytic leukemia, B-cell prolymphocytic leukemia, B-cell lymphoma (Greenwald, R. J., et al., “E mu-BRD2 transgenic mice develop B-cell lymphoma and leukemia,” Blood 103(4):1475-84 (2004)), basal cell carcinoma, biliary tract cancer, bladder cancer, blastoma, bone cancer (Lamoureux, F. et al. “Selective inhibition of BET bromodomain epigenetic signalling interferes with the bone-associated tumour vicious cycle,” Nature Comm 5:3511 (2014), Brenner tumor, Brown tumor, Burkitt's lymphoma (Mertz, J. A., et al., “Targeting MYC dependence in cancer by inhibiting BET bromodomains,” Proc Natl Acad Sci USA 108(40):16669-74 (2011)), breast cancer (Feng, Q. et al. “An epigenomic approach to therapy for tamoxifen-resistant breast cancer,” Cell Res 24:809-819 (2014); Nagarajan, S. et al. “Bromodomain Protein BRD4 is Required for Estrogen Receptor-Dependent Enhancer Activation and Gene Transcription,” Cell Rep 8:460-469 (2014); Shi, J. et al. “Disrupting the Interaction of BRD4 with Diacetylated Twist Suppresses Tumorigenesis in Basal-like Breast Cancer,” Cancer Cell 25:210-225 (2014)), brain cancer, carcinoma, carcinoma in situ, carcinosarcoma, cartilage tumor, cementoma, myeloid sarcoma, chondroma, chordoma, choriocarcinoma, choroid plexus papilloma, clear-cell sarcoma of the kidney, craniopharyngioma, cutaneous T-cell lymphoma, cervical cancer, colorectal cancer, Degos disease, desmoplastic small round cell tumor, diffuse large B-cell lymphoma (Chapuy, B. et al. “Discovery and characterization of super-enhancer-associated dependencies in diffuse large B cell lymphoma,” Cancer Cell 24:777-790 (2013); Trabucco, S. E. et al. “Inhibition of bromodomain proteins for the treatment of human diffuse large B-cell lymphoma,” Clin Can Res 21(1):113-122 (2015); Ceribelli, M. et al. “Blockade of oncogenic IkappaB kinase activity in diffuse large B-cell lymphoma by bromodomain and extraterminal domain protein inhibitors,” PNAS 111:11365-11370 (2014)), dysembryoplastic neuroepithelial tumor, dysgerminoma, embryonal carcinoma, endocrine gland neoplasm, endodermal sinus tumor, enteropathy-associated T-cell lymphoma, esophageal cancer, fetus in fetu, fibroma, fibrosarcoma, follicular lymphoma, follicular thyroid cancer, ganglioneuroma, gastrointestinal cancer, germ cell tumor, gestational choriocarcinoma, giant cell fibroblastoma, giant cell tumor of the bone, glial tumor, glioblastoma multiforme (Cheng, Z et al. “Inhibition of BET bromodomain targets genetically diverse glioblastoma,” Clin Con Res 19:1748-1759 (2013); Pastori, C. et al. “BET bromodomain proteins are required for glioblastoma cell proliferation,” Epigenetics 9:611-620 (2014)), glioma, gliomatosis cerebri, glucagonoma, gonadoblastoma, granulosa cell tumor, gynandroblastoma, gallbladder cancer, gastric cancer, hairy cell leukemia, hemangioblastoma, head and neck cancer, hemangiopericytoma, hematological malignancy, hepatoblastoma, hepatosplenic T-cell lymphoma, Hodgkin's lymphoma, non-Hodgkin's lymphoma (Lwin, T. et al. “A microenvironment-mediated c-Myc/miR-548m/HDAC6 amplification loop in non-Hodgkin B cell lymphomas,” J Clin Invest 123:4612-4626 (2013)), invasive lobular carcinoma, intestinal cancer, kidney cancer, laryngeal cancer, lentigo maligna, lethal midline carcinoma, leukemia, Leydig cell tumor, liposarcoma, lung cancer, lymphangioma, lymphangiosarcoma, lymphoepithelioma, lymphoma, acute lymphocytic leukemia, acute myelogenous leukemia (Mertz, J. A., et al., “Targeting MYC dependence in cancer by inhibiting BET bromodomains,” Proc Natl Acad Sci USA 108(40):16669-74 (2011)), chronic lymphocytic leukemia, liver cancer, small cell lung cancer, non-small cell lung cancer (Lockwood, W. W. et al. “Sensitivity of human lung adenocarcinoma cell lines to targeted inhibition of BET epigenetic signaling proteins,” PNAS 109:19408-19413 (2012); Shimamura, T. et al. “Efficacy of BET bromodomain inhibition in Kras-mutant non-small cell lung cancer,” Clin Can Res 19:6183-6192 (2013)) MALT lymphoma, malignant fibrous histiocytoma, malignant peripheral nerve sheath tumor (Baude, A. et al. “PRC2 loss amplifies Ras signaling in cancer,” Nat Genet 46:1154-1155 (2014); Patel, A J. et al. “BET bromodomain inhibition triggers apoptosis of NF1-associated malignant peripheral nerve sheath tumors through Bim induction,” Cell Rep 6:81-92 (2014)), malignant triton tumor, mantle cell lymphoma (Moros, A. et al. “Synergistic antitumor activity of lenalidomide with the BET bromodomain inhibitor CPI203 in bortezomib-resistant mantle cell lymphoma,” Leukemia 28:2049-2059 (2014)), marginal zone B-cell lymphoma, mast cell leukemia, mediastinal germ cell tumor, medullary carcinoma of the breast, medullary thyroid cancer, medulloblastoma (Bandopadhayay, P. et al. “BET bromodomain inhibition of MYC-amplified medulloblastoma,” Clin Can Res 20:912-925 (2014); Henssen, A. G. et al. “BET bromodomain protein inhibition is a therapeutic option for medulloblastoma” Oncotarget 4(11):2080-2089 (2013); Long, J. et al. “The BET bromodomain inhibitor I-BET151 acts downstream of Smoothened to abrogate the growth of Hedgehog driven cancers,” J Biol Chem 289(51):35494-35502 (2014); Tang, Y. et al. “Epigenetic targeting of Hedgehog pathway transcriptional output through BET bromodomain inhibition,” Nat Med 20(7):732-40 (2014); Venataraman, S. et al. “Inhibition of BRD4 attenuates tumor cell self-renewal and suppresses stem cell signaling in MYC driven medulloblastoma,” Oncotarget 5(9):2355-71 (2014)) melanoma (Segura et al, “BRD4 is a novel therapeutic target in melanoma,” Cancer Res 72(8): Supplement 1 (2012)), meningioma, Merkel cell cancer, mesothelioma, metastatic urothelial carcinoma, mixed Mullerian tumor, mixed lineage leukemia (Dawson, M. A., et al., “Inhibition of BET recruitment to chromatin as an effective treatment for MLL-fusion leukaemia,” Nature 478(7370):529-33 (2011)), mucinous tumor, multiple myeloma (Delmore, J. E., et al., “BET bromodomain inhibition as a therapeutic strategy to target c-Myc,” Cell 146(6):904-17 (2010)), muscle tissue neoplasm, mycosis fungoides, myxoid liposarcoma, myxoma, myxosarcoma, nasopharyngeal carcinoma, neurinoma, neuroblastoma (Puissant, A. et al. “Targeting MYCN in neuroblastoma by BET bromodomain inhibition,” Cancer Discov 3:308-323 (2013); Wyce, A. et al. “BET inhibition silences expression of MYCN and BCL2 and induces cytotoxicity in neuroblastoma tumor models,” PLoS One 8:e72967 (2014)), neurofibroma, neuroma, nodular melanoma, NUT-midline carcinoma (Filippakopoulos, P., et al., “Selective inhibition of BET bromodomains,” Nature 468(7327):1067-73 (2010)), ocular cancer, oligoastrocytoma, oligodendroglioma, oncocytoma, optic nerve sheath meningioma, optic nerve tumor, oral cancer, osteosarcoma (Lamoureux, F. et al. “Selective inhibition of BET bromodomain epigenetic signalling interferes with the bone-associated tumour vicious cycle” Nat Commun 5:3511 (2014); Lee, D. H. et al. “Synergistic effect of JQ1 and rapamycin for treatment of human osteosarcoma,” Int J Cancer 136(9):2055-2064 (2014)), ovarian cancer, Pancoast tumor, papillary thyroid cancer, paraganglioma, pinealoblastoma, pineocytoma, pituicytoma, pituitary adenoma, pituitary tumor, plasmacytoma, polyembryoma, precursor T-lymphoblastic lymphoma, primary central nervous system lymphoma, primary effusion lymphoma (Tolani, B. et al. “Targeting Myc in KSHV-associated primary effusion lymphoma with BET bromodomain inhibitors,” Oncogene 33:2928-2937 (2014)), primary peritoneal cancer, prostate cancer (Asangani, I. A. et al. “Therapeutic targeting of BET bromodomain proteins in castration-resistant prostate cancer,” Nature 510:278-282 (2014); Cho, H. et al. “RapidCaP, a novel GEM model for metastatic prostate cancer analysis and therapy, reveals myc as a driver of Pten-mutant metastasis,” Cancer Discov 4:318-333 (2014); Gao, L. et al. “Androgen receptor promotes ligand-independent prostate cancer progression through c-Myc upregulation,” PLoS One 8:e63563 (2013): Wyce, A. et al. “Inhibition of BET bromodomain proteins as a therapeutic approach in prostate cancer,” Oncotarget 4:2419-2429 (2013)), pancreatic cancer (Sahai, V. et al. “BET bromodomain inhibitors block growth of pancreatic cancer cells in three-dimensional collagen,” Mol Cancer Ther 13:1907-1917 (2014)), pharyngeal cancer, pseudomyxoma peritonei, renal cell carcinoma, renal medullary carcinoma, retinoblastoma, rhabdomyoma, rhabdomyosarcoma, Richter's transformation, rectal cancer, sarcoma, Schwannomatosis, seminoma, Sertoli cell tumor, sex cord-gonadal stromal tumor, signet ring cell carcinoma, skin cancer, small blue round cell tumors, small cell carcinoma, soft tissue sarcoma, somatostatinoma, soot wart, spinal tumor, splenic marginal zone lymphoma, squamous cell carcinoma, synovial sarcoma, Sezary's disease, small intestine cancer, squamous carcinoma, stomach cancer, testicular cancer, thecoma, thyroid cancer, transitional cell carcinoma, throat cancer, urachal cancer, urogenital cancer, urothelial carcinoma, uveal melanoma, uterine cancer, verrucous carcinoma, visual pathway glioma, vulvar cancer, vaginal cancer, Waldenstrom's macroglobulinemia, Warthin's tumor, and Wilms' tumor. Thus, one aspect of the inventions provides compounds, compositions, and methods for treating such cancers.

BET inhibitors of the invention may be useful in the treatment of cancers that are resistant to current and future cancer treatments, as BET proteins are involved in the mechanisms of resistance of several anti-cancer treatment, including chemotherapy (Feng, Q., et al. “An epigenomic approach to therapy for tamoxifen-resistant breast cancer” Cell Res 24:809-819 (2014)), immunotherapy (Emadali, A., et al. “Identification of a novel BET bromodomain inhibitor-sensitive, gene regulatory circuit that controls Rituximab response and tumour growth in aggressive lymphoid cancers,” EMBO Mol Med 5:1180-1195 (2013)), hormone-deprivation therapies (Asangani, I. A. et al. “Therapeutic targeting of BET bromodomain proteins in castration-resistant prostate cancer,” Nature 510:278-282 (2014)), or other molecules ((Knoechel, B. et al. “An epigenetic mechanism of resistance to targeted therapy in T cell acute lymphoblastic leukemia,” Not Genet 46:364-370 (2014)). In these instances, the BET proteins are involved in the resistance mechanism to the cancer therapy, and treatment with a BET inhibitor could either restore sensitivity to the treatment, inhibit proliferation or induce cell death or senescence, either alone or in combination with other therapies (Moros, A. et al. “Synergistic antitumor activity of lenalidomide with the BET bromodomain inhibitor CPI203 in bortezomib-resistant mantle cell lymphoma,” Leukemia 28:2049-2059 (2014)).

BET inhibitors may be useful in the treatment of benign proliferative and fibrotic disorders, including benign soft tissue tumors, bone tumors, brain and spinal tumors, eyelid and orbital tumors, granuloma, lipoma, meningioma, multiple endocrine neoplasia, nasal polyps, pituitary tumors, prolactinoma, pseudotumor cerebri, seborrheic keratoses, stomach polyps, thyroid nodules, cystic neoplasms of the pancreas, hemangiomas, vocal cord nodules, polyps, and cysts, Castleman disease, chronic pilonidal disease, dermatofibroma, pilar cyst, pyogenic granuloma, juvenile polyposis syndrome, idiopathic pulmonary fibrosis, renal fibrosis, post-operative stricture, keloid formation, scleroderma, and cardiac fibrosis. Tang, X. et al., “Assessment of Brd4 Inhibition in Idiopathic Pulmonary Fibrosis Lung Fibroblasts and in Vivo Models of Lung Fibrosis,” Am J Pathology 183(2):470-479 (2013). Thus, one aspect of the invention provides compounds, compositions, and methods for treating such benign proliferative and fibrotic disorders.

Cardiovascular disease (CVD) is the leading cause of mortality and morbidity in the United States. Roger, V. L, et al., “Heart disease and stroke statistics-2012 update: a report from the American Heart Association,” Circulation 125(1):e2-e220 (2012). Atherosclerosis, an underlying cause of CVD, is a multifactorial disease characterized by dyslipidemia and inflammation. BET inhibitors are expected to be efficacious in atherosclerosis and associated conditions because of aforementioned anti-inflammatory effects as well as ability to increase transcription of ApoA-I, the major constituent of HDL. Mirguet, O., et al., “From ApoA1 upregulation to BET family bromodomain inhibition: discovery of I-BET151,” Bioorg Med Chem Lett 22(8):2963-7 (2012); Chung, C. W., et al., “Discovery and characterization of small molecule inhibitors of the BET family bromodomains,” J Med Chem 54(11):3827-38 (2011). Accordingly, one aspect of the invention provides compounds, compositions, and methods for treating cardiovascular disease, including but not limited to atherosclerosis.

Up-regulation of ApoA-I is considered to be a useful strategy in treatment of atherosclerosis and CVD. Degoma, E. M. and D. J. Rader, “Novel HDL-directed pharmacotherapeutic strategies,” Nat Rev Cardiol 8(5):266-77 (2011). BET inhibitors have been shown to increase ApoA-I transcription and protein expression. Mirguet, O., et al., “From ApoA1 upregulation to BET family bromodomain inhibition: discovery of I-BET151,” Bioorg Med Chem Lett 22(8):2963-7 (2012); Chung, C. W., et al., “Discovery and characterization of small molecule inhibitors of the BET family bromodomains,” J Med Chem 54(11):3827-38 (2011). It has also been shown that BET inhibitors bind directly to BET proteins and inhibit their binding to acetylated histones at the ApoA-1 promoter, suggesting the presence of a BET protein repression complex on the ApoA-1 promoter, which can be functionally disrupted by BET inhibitors. It follows that, BET inhibitors may be useful in the treatment of disorders of lipid metabolism via the regulation of ApoA-I and HDL such as hypercholesterolemia, dyslipidemia, atherosclerosis (Degoma, E. M. and D J. Rader, “Novel HDL-directed pharmacotherapeutic strategies,” Nat Rev Cordial 8(5):266-77 (2011)), and Alzheimer's disease and other neurological disorders. Elliott, D. A., et al., “Apolipoproteins in the brain: implications for neurological and psychiatric disorders,” Clin Lipidol 51(4):555-573 (2010). Thus, one aspect of the invention provides compounds, compositions, and methods for treating cardiovascular disorders by upregulation of ApoA-1.

BET inhibitors may be useful in the prevention and treatment of conditions associated with ischemia-reperfusion injury such as, but not limited to, myocardial infarction, stroke, acute coronary syndromes (Prinjha, R. K., J. Witherington, and K. Lee, “Place your BETs: the therapeutic potential of bromodomains,” Trends Pharmacol Sci 33(3):146-53 (2012)), renal reperfusion injury, organ transplantation, coronary artery bypass grafting, cardio-pulmonary bypass procedures, hypertension, pulmonary, renal, hepatic, gastro-intestinal, or peripheral limb embolism. Accordingly, one aspect of the invention provides compounds, compositions, and methods for prevention and treatment of conditions described herein that are associated with ischemia-reperfusion injury.

Obesity-associated inflammation is a hallmark of type II diabetes, insulin resistance, and other metabolic disorders. Belkina, A. C. and G. V. Denis, “BET domain co-regulators in obesity, inflammation and cancer,” Nat Rev Cancer 12(7):465-77 (2012); Denis, G. V., “Bromodomain coactivators in cancer, obesity, type 2 diabetes, and inflammation,” Discov Med 10(55):489-99 (2010). Consistent with the ability of BET inhibitors to inhibit inflammation, gene disruption of Brd2 in mice ablates inflammation and protects animals from obesity-induced insulin resistance. Wang, F., et al., “Brd2 disruption in mice causes severe obesity without Type 2 diabetes,” Biochem J 425(1):71-83 (2010). It has been shown that Brd2 interacts with PPARγ and opposes its transcriptional function. Knockdown of Brd2 in vitro promotes transcription of PPARγ-regulated networks, including those controlling adipogenesis. Denis, G. V., et al, “An emerging role for bromodomain-containing proteins in chromatin regulation and transcriptional control of adipogenesis,” FEBS Lett 584(15):3260-8 (2010). In addition Brd2 is highly expressed in pancreatic l-cells and regulates proliferation and insulin transcription. Wang, F., et al., “Brd2 disruption in mice causes severe obesity without Type 2 diabetes,” Biochem J 425(1):71-83 (2010). Taken together, the combined effects of BET inhibitors on inflammation and metabolism decrease insulin resistance and may be useful in the treatment of pre-diabetic and type II diabetic individuals as well as patients with other metabolic complications. Belkina, A. C. and G. V. Denis, “BET domain co-regulators in obesity, inflammation and cancer,” Nat Rev Cancer 12(7):465-77 (2012). Accordingly, one aspect of the invention provides compounds, compositions, and methods for treatment and prevention of metabolic disorders, including but not limited to obesity-associated inflammation, type II diabetes, and insulin resistance.

BET inhibitors may be useful in the prevention and treatment of episome-based DNA viruses including, but not limited to, human papillomavirus, herpes virus, Epstein-Barr virus, human immunodeficiency virus (Belkina, A. C. and G. V. Denis, “BET domain co-regulators in obesity, inflammation and cancer,” Nat Rev Cancer 12(7):465-77 (2012)), adenovirus, poxvirus, hepatitis B virus, and hepatitis C virus. Host-encoded BET proteins have been shown to be important for transcriptional activation and repression of viral promoters. Brd4 interacts with the E2 protein of human papilloma virus (HPV) to enable E2 mediated transcription of E2-target genes. Gagnon, D., et al., “Proteasomal degradation of the papillomavirus E2 protein is inhibited by overexpression of bromodomain-containing protein 4,” J Viral 83(9):4127-39 (2009). Similarly, Brd2, Brd3, and Brd4 all bind to latent nuclear antigen 1 (LANA1), encoded by Kaposi's sarcoma-associated herpes virus (KSHV), promoting LANA1-dependent proliferation of KSHV-infected cells. You, J., et al., “Kaposi's sarcoma-associated herpesvirus latency-associated nuclear antigen interacts with bromodomain protein Brd4 on host mitotic chromosomes,” J Viral 80(18):8909-19 (2006). A BET inhibitor has been shown to inhibit the Brd4-mediated recruitment of the transcription elongation complex pTEFb to the Epstein-Barr virus (EBV) viral C promoter, suggesting therapeutic value for EBV-associated malignancies. Palermo, R. D., et al., “RNA polymerase II stalling promotes nucleosome occlusion and pTEFb recruitment to drive immortalization by Epstein-Barr virus,” PLoS Pathog 7(10):e1002334 (2011). Also, a BET inhibitor reactivated HIV in models of latent T cell infection and latent monocyte infection, potentially allowing for viral eradication by complementary anti-retroviral therapy. Zhu, J., et al., “Reactivation of Latent HIV-1 by Inhibition of BRD4,” Cell Rep 2(4):807-816 (2012); Banerjee, C., et al., “BET bromodomain inhibition as a novel strategy for reactivation of HIV-1,” J Leukoc Biol 92(6):1147-1154 (2012); Bartholomeeusen, K., et al., “BET bromodomain inhibition activates transcription via a transient release of P-TEFb from 7SK snRNP,” J Biol Chem 287(43):36609-36616 (2012); Li, Z., et al., “The BET bromodomain inhibitor JQ1 activates HIV latency through antagonizing Brd4 inhibition of Tat-transactivation,” Nucleic Acids Res (2012). Thus, the invention also provides compounds, compositions, and methods for treatment and prevention of episome-based DNA virus infections. In particular, one aspect of the invention provides compounds, compositions, and methods for treatment and/or prevention of a viral infection, including, but not limited to infection by HPV, KSHV, EBV, HIV, HBV, HCV, adenovirus, poxvirus herpes virus, or a malignancy associated with that infection.

Some central nervous system (CNS) diseases are characterized by disorders in epigenetic processes. Brd2 haplo-insufficiency has been linked to neuronal deficits and epilepsy. Velisek, L., et al., “GABAergic neuron deficit as an idiopathic generalized epilepsy mechanism: the role of BRD2 haploinsufficiency in juvenile myoclonic epilepsy,” PLoS One 6(8): e23656 (2011). SNPs in various bromodomain-containing proteins have also been linked to mental disorders including schizophrenia and bipolar disorders. Prinjha, R. K., J. Witherington, and K. Lee, “Place your BETs: the therapeutic potential of bromodomains,” Trends Pharmacol Sci 33(3):146-53 (2012). In addition, the ability of BET inhibitors to increase ApoA-I transcription may make BET inhibitors useful in Alzheimer's disease therapy considering the suggested relationship between increased ApoA-I and Alzheimer's disease and other neurological disorders. Elliott, D. A., et al., “Apolipoproteins in the brain: implications for neurological and psychiatric disorders,” Clin Lipidol 51(4):555-573 (2010). Accordingly, one aspect of the invention provides compounds, compositions, and methods for treating such CNS diseases and disorders.

BRDT is the testis-specific member of the BET protein family which is essential for chromatin remodeling during spermatogenesis. Gaucher, J., et al., “Bromodomain-dependent stage-specific male genome programming by Brdt,” EMBO J 31(19):3809-20 (2012); Shang, E., et al., “The first bromodomain of Brdt, a testis-specific member of the BET sub-family of double-bromodomain-containing proteins, is essential for male germ cell differentiation,” Development 134(19):3507-15 (2007). Genetic depletion of BRDT or inhibition of BRDT interaction with acetylated histones by a BET inhibitor resulted in a contraceptive effect in mice, which was reversible when small molecule BET inhibitors were used. Matzuk, M. M., et al., “Small-Molecule Inhibition of BRDT for Male Contraception,” Cell 150(4):673-684 (2012); Berkovits, B. D., et al., “The testis-specific double bromodomain-containing protein BRDT forms a complex with multiple spliceosome components and is required for mRNA splicing and 3′-UTR truncation in round spermatids,” Nucleic Acids Res 40(15):7162-75 (2012). These data suggest potential utility of BET inhibitors as a novel and efficacious approach to male contraception. Thus, another aspect of the invention provides compounds, compositions, and methods for male contraception.

Monocyte chemotactic protein-1 (MCP-1, CCL2) plays an important role in cardiovascular disease. Niu, J. and P. E. Kolattukudy, “Role of MCP-1 in cardiovascular disease: molecular mechanisms and clinical implications,” Clin Sci (Lond) 117(3):95-109 (2009). MCP-1, by its chemotactic activity, regulates recruitment of monocytes from the arterial lumen to the subendothelial space, where they develop into macrophage foam cells, and initiate the formation of fatty streaks which can develop into atherosclerotic plaque. Dawson, J., et al., “Targeting monocyte chemoattractant protein-1 signalling in disease,” Expert Opin Ther Targets 7(1):35-48 (2003). The critical role of MCP-1 (and its cognate receptor CCR2) in the development of atherosclerosis has been examined in various transgenic and knockout mouse models on a hyperlipidemic background. Boring, L, et al., “Decreased lesion formation in CCR2−/− mice reveals a role for chemokines in the initiation of atherosclerosis,” Nature 394(6696):894-7 (1998); Gosling, J., et al., “MCP-1 deficiency reduces susceptibility to atherosclerosis in mice that overexpress human apolipoprotein B,” J Clin Invest 103(6):773-8 (1999); Gu, L., et al., “Absence of monocyte chemoattractant protein-1 reduces atherosclerosis in low density lipoprotein receptor-deficient mice,” Mol Cell 2(2):275-81 (1998); Aiello, R J., et al., “Monocyte chemoattractant protein-1 accelerates atherosclerosis in apolipoprotein E-deficient mice,” Arterioscler Thromb Vasc Biol 19(6):1518-25 (1999). These reports demonstrate that abrogation of MCP-1 signaling results in decreased macrophage infiltration to the arterial wall and decreased atherosclerotic lesion development.

The association between MCP-1 and cardiovascular disease in humans is well-established. Niu, J. and P. E. Kolattukudy, “Role of MCP-1 in cardiovascular disease: molecular mechanisms and clinical implications,” Clin Sci (Lond) 117(3):95-109 (2009). MCP-1 and its receptor are overexpressed by endothelial cells, smooth muscle cells, and infiltrating monocytes/macrophages in human atherosclerotic plaque. Nelken, N. A., et al., “Monocyte chemoattractant protein-1 in human atheromatous plaques,” J Clin Invest 88(4):1121-7 (1991). Moreover, elevated circulating levels of MCP-1 are positively correlated with most cardiovascular risk factors, measures of coronary atherosclerosis burden, and the incidence of coronary heart disease (CHD). Deo, R., et al., “Association among plasma levels of monocyte chemoattractant protein-1, traditional cardiovascular risk factors, and subclinical atherosclerosis,” J Am Coll Cardiol 44(9):1812-8 (2004). CHD patients with among the highest levels of MCP-1 are those with acute coronary syndrome (ACS). de Lemos, J. A., et al., “Association between plasma levels of monocyte chemoattractant protein-1 and long-term clinical outcomes in patients with acute coronary syndromes,” Circulation 107(5):690-5 (2003). In addition to playing a role in the underlying inflammation associated with CHD, MCP-1 has been shown to be involved in plaque rupture, ischemic/reperfusion injury, restenosis, and heart transplant rejection. Niu, J. and P. E. Kolattukudy, “Role of MCP-1 in cardiovascular disease: molecular mechanisms and clinical implications,” Clin Sci (Lond) 117(3):95-109 (2009).

MCP-1 also promotes tissue inflammation associated with autoimmune diseases including rheumatoid arthritis (RA) and multiple sclerosis (MS). MCP-1 plays a role in the infiltration of macrophages and lymphocytes into the joint in RA, and is overexpressed in the synovial fluid of RA patients. Koch, A. E., et al., “Enhanced production of monocyte chemoattractant protein-1 in rheumatoid arthritis,” J Clin Invest 90(3):772-9 (1992). Blockade of MCP-1 and MCP-1 signaling in animal models of RA have also shown the importance of MCP-1 to macrophage accumulation and proinflammatory cytokine expression associated with RA. Brodmerkel, C. M., et al., “Discovery and pharmacological characterization of a novel rodent-active CCR2 antagonist, INCB3344,” J Immunol 175(8):5370-8 (2005); Bruhl, H., et al., “Dual role of CCR2 during initiation and progression of collagen-induced arthritis: evidence for regulatory activity of CCR2+ T cells,” J Immunol 172(2):890-8 (2004); Gong, J. H., et al., “An antagonist of monocyte chemoattractant protein 1 (MCP-1) inhibits arthritis in the MRL-lpr mouse model,” J Exp Med 186(1):131-7 (1997); 65. Gong, J. H., et al., “Post-onset inhibition of murine arthritis using combined chemokine antagonist therapy,” Rheumatology (Oxford 43(1): 39-42 (2004).

Overexpression of MCP-1, in the brain, cerebrospinal fluid (CSF), and blood, has also been associated with chronic and acute MS in humans. Mahad, D. J. and R. M. Ransohoff, “The role of MCP-1 (CCL2) and CCR2 in multiple sclerosis and experimental autoimmune encephalomyelitis (EAE),” Semin Immunol 15(1):23-32 (2003). MCP-1 is overexpressed by a variety of cell types in the brain during disease progression and contributes to the infiltration of macrophages and lymphocytes which mediate the tissue damage associated with MS. Genetic depletion of MCP-1 or CCR2 in the experimental autoimmune encephalomyelitis (EAE) mouse model, a model resembling human MS, results in resistance to disease, primarily because of decreased macrophage infiltration to the CNS. Fife, B. T., et al., “CC chemokine receptor 2 is critical for induction of experimental autoimmune encephalomyelitis,” J Exp Med 192(6):899-905 (2000); Huang, D. R., et al., “Absence of monocyte chemoattractant protein 1 in mice leads to decreased local macrophage recruitment and antigen-specific T helper cell type 1 immune response in experimental autoimmune encephalomyelitis,” J Exp Med 193(6):713-26 (2001).

Preclinical data have suggested that small- and large-molecule inhibitors of MCP-1 and CCR2 have potential as therapeutic agents in inflammatory and autoimmune indications. Thus, one aspect of the invention provides compounds, compositions, and methods for treating cardiovascular, inflammatory, and autoimmune conditions associated with MCP-1 and CCR2.

Accordingly, the invention provides compounds that are useful for inhibition of BET protein function by binding to bromodomains, pharmaceutical compositions comprising one or more of those compounds, and use of these compounds or compositions in the treatment and prevention of diseases and conditions, including, but not limited to, cancer, autoimmune, and cardiovascular diseases.

One aspect of the invention includes compounds of Formula I:

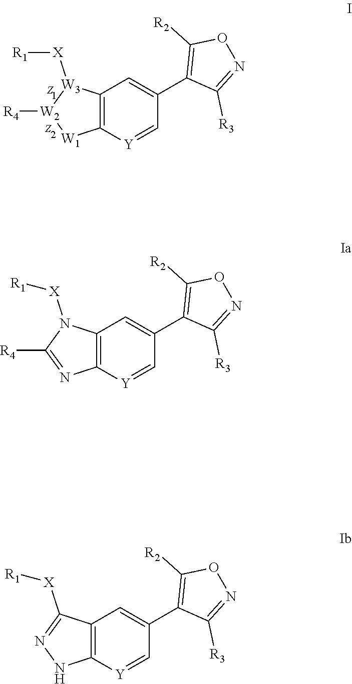

and stereoisomers, tautomers, pharmaceutically acceptable salts, and hydrates thereof,
wherein:
W₁ is N or NH;
W₂ and W₃ are selected from C and N;
Z₁ and Z₂ are selected from a single bond and double bond;
R₁ is selected from carbocycle (C₅-C₁₀) and heterocycle (C₂-C₁₀) optionally substituted with 1 to 5 groups independently selected from R₅;
R₂ is selected from hydrogen, alkyl (C₁-C₆) optionally substituted with halogen and hydroxyl;
R₃ is selected from alkyl (C₁-C₆) optionally substituted with halogen and hydroxyl, with the proviso that if R₂ and R₃ are methyls, then R₁ is different from:

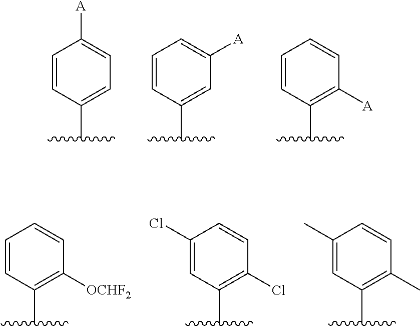

where A is selected from hydrogen, halogen, methoxy, —CN, —NO₂, —COOMe, and —CONMe₂;
R₄ if present, is selected from hydrogen, alkyl (C₁-C₁₀), carbocycle (C₃-C₁₀) and heterocycle (C₂-C₁₀) optionally substituted with 1 to 5 groups independently selected from R₅;
each R₅ is independently selected from deuterium, alkyl(C₁-C₆) (such as, e.g., methyl, ethyl, propyl, isopropyl, butyl), alkoxy(C₁-C₆) (such as, e.g., methoxy, ethoxy, isopropoxy), amino (such as, e.g., —NH₂, —NHMe, —NHEt, —NHiPr, —NHBu —NMe₂, NMeEt, —NEt₂, —NEtBu), —NHC(O)NH-alkyl(C₁-C₆), halogen (such as, e.g., F, Cl), amide (such as, e.g., —NHC(O)Me, —NHC(O)Et, —C(O)NHMe, —C(O)NEt₂, —C(O)NiPr), —CF₃, —CN, —N₃, ketone (C₁-C₆) (such as, e.g., acetyl, —C(O)Et, —C(O)Pr), —S(O)-alkyl(C₁-C₄) (such as, e.g., —S(O)Me, —S(O)Et), —SO₂-alkyl(C₁-C₆) (such as, e.g., —SO₂Me, —SO₂Et, —SO₂Pr), thioalkyl(C₁-C₆) (such as, e.g., —SMe, —SEt, —SPr, —SBu), —COOH, and ester (such as, e.g., —C(O)OMe, —C(O)OEt, —C(O)OBu), each of which may be optionally substituted with hydrogen, F, Cl, Br, —OH, —NH₂, —NHMe, —OMe, —SMe, oxo, and/or thio-oxo;
X is selected from —CH₂— optionally substituted with 1 to 2 groups independently selected from R₅;
Y is selected from N and CH;
wherein if W₃ is C then W₂ is N, W₁ is NH, Z₁ is a double bond, Z₂ is a single bond, and R₄ is absent; and
wherein if W₃ is N then W₂ is C, W₁ is N, Z₁ is a single bond, and Z₂ is a double bond.

In another aspect of the invention, a pharmaceutical composition comprising a compound of Formula I or a stereoisomer, tautomer, pharmaceutically acceptable salt, or hydrate thereof and one or more pharmaceutically acceptable carrier, diluent or excipient is provided.

In yet another aspect of the invention there is provided a compound of Formula I or a stereoisomer, tautomer, pharmaceutically acceptable salt, or hydrate thereof for use in therapy, in particular in the treatment of diseases or conditions for which a bromodomain inhibitor is indicated. Thus, one aspect of the invention comprises administering a therapeutically effective amount of a compound of Formula I, including a compound of Formula Ia or Formula Ib, or a stereoisomer, tautomer, pharmaceutically acceptable salt, or hydrate thereof, to a mammal (e.g., a human) in need thereof.

Another aspect of the invention provides methods of administering a therapeutically effective amount of a compound of Formula I or a stereoisomer, tautomer, pharmaceutically acceptable salt, or hydrate thereof, to a mammal (e.g., a human) in need thereof.

Another aspect of the invention provides for the use of a compound of Formula I or a stereoisomer, tautomer, pharmaceutically acceptable salt, or hydrate thereof in the manufacture of a medicament for the treatment of diseases or conditions for which a bromodomain inhibitor is indicated.

### Definitions

As used in the present specification, the following words, phrases and symbols are generally intended to have the meanings as set forth below, except to the extent that the context in which they are used indicates otherwise. The following abbreviations and terms have the indicated meanings throughout.

As used herein, “cardiovascular disease” refers to diseases, disorders and conditions of the heart and circulatory system that are mediated by BET inhibition. Exemplary cardiovascular diseases, including cholesterol- or lipid-related disorders, include, but are not limited to, acute coronary syndrome, angina, arteriosclerosis, atherosclerosis, carotid atherosclerosis, cerebrovascular disease, cerebral infarction, congestive heart failure, congenital heart disease, coronary heart disease, coronary artery disease, coronary plaque stabilization, dyslipidemias, dyslipoproteinemias, endothelium dysfunctions, familial hypercholesterolemia, familial combined hyperlipidemia, hypoalphalipoproteinemia, hypertriglyceridemia, hyperbetalipoproteinemia, hypercholesterolemia, hypertension, hyperlipidemia, intermittent claudication, ischemia, ischemia reperfusion injury, ischemic heart diseases, cardiac ischemia, metabolic syndrome, multi-infarct dementia, myocardial infarction, obesity, peripheral vascular disease, reperfusion injury, restenosis, renal artery atherosclerosis, rheumatic heart disease, stroke, thrombotic disorder, transitory ischemic attacks, and lipoprotein abnormalities associated with Alzheimer's disease, obesity, diabetes mellitus, syndrome X, and impotence.

As used herein, “inflammatory diseases” refers to diseases, disorders, and conditions that are mediated by BET inhibition. Exemplary inflammatory diseases, include, but are not limited to, arthritis, asthma, dermatitis, psoriasis, cystic fibrosis, post transplantation late and chronic solid organ rejection, multiple sclerosis, systemic lupus erythematosus, inflammatory bowel diseases, autoimmune diabetes, diabetic retinopathy, diabetic nephropathy, diabetic vasculopathy, ocular inflammation, uveitis, rhinitis, ischemia-reperfusion injury, post-angioplasty restenosis, chronic obstructive pulmonary disease (COPD), glomerulonephritis, Graves disease, gastrointestinal allergies, conjunctivitis, atherosclerosis, coronary artery disease, angina, and small artery disease.

As used herein, “cancer” refers to diseases, disorders, and conditions that are mediated by BET inhibition. Exemplary cancers, include, but are not limited to, chronic lymphocytic leukemia and multiple myeloma, follicular lymphoma, diffuse large B cell lymphoma with germinal center phenotype, Burkitt's lymphoma, Hodgkin's lymphoma, follicular lymphomas and activated, anaplastic large cell lymphoma, neuroblastoma and primary neuroectodermal tumor, rhabdomyosarcoma, prostate cancer, breast cancer, NMC (NUT-midline carcinoma), acute myeloid leukemia (AML), acute B lymphoblastic leukemia (B-ALL), Burkitt's Lymphoma, B-cell lymphoma, melanoma, mixed lineage leukemia, multiple myeloma, pro-myelocytic leukemia (PML), non-Hodgkin's lymphoma, neuroblastoma, medulloblastoma, lung carcinoma (NSCLC, SCLC), and colon carcinoma.

“Subject” refers to an animal, such as a mammal, that has been or will be the object of treatment, observation, or experiment. The methods described herein may be useful for both human therapy and veterinary applications. In one embodiment, the subject is a human.

As used herein, “treatment” or “treating” refers to an amelioration of a disease or disorder, or at least one discernible symptom thereof. In another embodiment, “treatment” or “treating” refers to an amelioration of at least one measurable physical parameter, not necessarily discernible by the patient. In yet another embodiment, “treatment” or “treating” refers to inhibiting the progression of a disease or disorder, either physically, e.g., stabilization of a discernible symptom, physiologically, e.g., stabilization of a physical parameter, or both. In yet another embodiment, “treatment” or “treating” refers to delaying the onset of a disease or disorder. For example, treating a cholesterol disorder may comprise decreasing blood cholesterol levels.

As used herein, “prevention” or “preventing” refers to a reduction of the risk of acquiring a given disease or disorder.

A dash (“-”) that is not between two letters or symbols is used to indicate a point of attachment for a substituent. For example, —CONH₂ is attached through the carbon atom.

By “optional” or “optionally” is meant that the subsequently described event or circumstance may or may not occur, and that the description includes instances where the event or circumstance occurs and instances in which is does not. For example, “optionally substituted aryl” encompasses both “aryl” and “substituted aryl” as defined below. It will be understood by those skilled in the art, with respect to any group containing one or more substituents, that such groups are not intended to introduce any substitution or substitution patterns that are sterically impractical, synthetically non-feasible and/or inherently unstable.

As used herein, the term “hydrate” refers to a crystal form with either a stoichiometric or non-stoichiometric amount of water is incorporated into the crystal structure.

The term “alkenyl” as used herein refers to an unsaturated straight or branched hydrocarbon having at least one carbon-carbon double bond, such as, e.g., a straight or branched group of 2-8 carbon atoms, referred to herein as (C₂-C₈)alkenyl. Exemplary alkenyl groups include, but are not limited to, vinyl, allyl, butenyl, pentenyl, hexenyl, butadienyl, pentadienyl, hexadienyl, 2-ethylhexenyl, 2-propyl-2-butenyl, and 4-(2-methyl-3-butene)-pentenyl.

The term “alkoxy” as used herein refers to an alkyl group attached to an oxygen (—O— alkyl-). “Alkoxy” groups also include an alkenyl group attached to an oxygen (“alkenyloxy”) or an alkynyl group attached to an oxygen (“alkynyloxy”) groups. Exemplary alkoxy groups include, but are not limited to, groups with an alkyl, alkenyl or alkynyl group of 1-8 carbon atoms, referred to herein as (C₁-C₈)alkoxy. Exemplary alkoxy groups include, but are not limited to methoxy and ethoxy.

The term “alkyl” as used herein refers to a saturated straight or branched hydrocarbon, such as, e.g., a straight or branched group of 1-8 carbon atoms, referred to herein as (C₁. Ca)alkyl. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, and octyl.

The term “alkynyl” as used herein refers to an unsaturated straight or branched hydrocarbon having at least one carbon-carbon triple bond, such as, e.g., a straight or branched group of 2-8 carbon atoms, referred to herein as (C₂-C₈)alkynyl. Exemplary alkynyl groups include, but are not limited to, ethynyl, propynyl, butynyl, pentynyl, hexynyl, methylpropynyl, 4-methyl-1-butynyl, 4-propyl-2-pentynyl, and 4-butyl-2-hexynyl.

The term “amide” as used herein refers to the form —NRₐC(O)(R_{b})— or —C(O)NR_{b}R_{c}, wherein Rₐ, R_{b} and R_{c} are each independently selected from alkyl, alkenyl, alkynyl, aryl, arylalkyl, cycloalkyl, haloalkyl, heteroaryl, heterocyclyl, and hydrogen. The amide can be attached to another group through the carbon, the nitrogen, R_{b}, or R_{c}. The amide also may be cyclic, for example R_{b} and R_{c}, may be joined to form a 3- to 8-membered ring, such as, e.g., 5- or 6-membered ring. The term “amide” encompasses groups such as, e.g., sulfonamide, urea, ureido, carbamate, carbamic acid, and cyclic versions thereof. The term “amide” also encompasses an amide group attached to a carboxy group, e.g., -amide-COOH or salts such as, e.g., -amide-COONa, an amino group attached to a carboxy group (e.g., -amino-COOH or salts such as, e.g., -amino-COONa).

The term “amine” or “amino” as used herein refers to the form —NR_{d}Rₑ or —N(R_{d})Rₑ—, where R_{d} and Rₑ are independently selected from alkyl, alkenyl, alkynyl, aryl, arylalkyl, carbamate, cycloalkyl, haloalkyl, heteroaryl, heterocycle, and hydrogen. The amino can be attached to the parent molecular group through the nitrogen. The amino also may be cyclic, for example any two of R_{d} and Rₑ may be joined together or with the N to form a 3- to 12-membered ring (e.g., morpholino or piperidinyl). The term amino also includes the corresponding quaternary ammonium salt of any amino group. Exemplary amino groups include alkylamino groups, wherein at least one of R_{d} or Rₑ is an alkyl group. In some embodiments R_{d} and Rₑ each may be optionally substituted with hydroxyl, halogen, alkoxy, ester, or amino.

The term “aryl” as used herein refers to a mono-, bi-, or other multi-carbocyclic, aromatic ring system. The aryl group can optionally be fused to one or more rings selected from aryls, cycloalkyls, and heterocyclyls. The aryl groups of this present disclosure can be substituted with groups selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone. Exemplary aryl groups include, but are not limited to, phenyl, tolyl, anthracenyl, fluorenyl, indenyl, azulenyl, and naphthyl, as well as benzo-fused carbocyclic moieties such as, e.g., 5,6,7,8-tetrahydronaphthyl. Exemplary aryl groups also include, but are not limited to a monocyclic aromatic ring system, wherein the ring comprises 6 carbon atoms, referred to herein as “(C₆)aryl.”

The term “arylalkyl” as used herein refers to an alkyl group having at least one aryl substituent (e.g., -aryl-alkyl-). Exemplary arylalkyl groups include, but are not limited to, arylalkyls having a monocyclic aromatic ring system, wherein the ring comprises 6 carbon atoms, referred to herein as “(C₆)arylalkyl.”

The term “carbamate” as used herein refers to the

form —R_{g}OC(O)N(Rₕ)—, —R_{g}OC(O)N(Rₕ)Rᵢ—, or —OC(O)NRₕRᵢ, wherein R_{g}, Rₕ and Rᵢ are each independently selected from alkyl, alkenyl, alkynyl, aryl, arylalkyl, cycloalkyl, haloalkyl, heteroaryl, heterocyclyl, and hydrogen. Exemplary carbamates include, but are not limited to, arylcarbamates or heteroaryl carbamates (e.g., wherein at least one of R_{g}, Rₕ and Rᵢ are independently selected from aryl or heteroaryl, such as, e.g., pyridine, pyridazine, pyrimidine, and pyrazine).

The term “carbocycle” as used herein refers to an aryl or cycloalkyl group.

The term “carboxy” as used herein refers to —COOH or its corresponding carboxylate salts (e.g., —COONa). The term carboxy also includes “carboxycarbonyl,” e.g. a carboxy group attached to a carbonyl group, e.g., —C(O)—COOH or salts, such as, e.g., —C(O)—COONa.

The term “cyano” as used herein refers to —CN.

The term “cycloalkoxy” as used herein refers to a cycloalkyl group attached to an oxygen.

The term “cycloalkyl” as used herein refers to a saturated or unsaturated cyclic, bicyclic, or bridged bicyclic hydrocarbon group of 3-12 carbons, or 3-8 carbons, referred to herein as “(C₃—C)cycloalkyl,” derived from a cycloalkane. Exemplary cycloalkyl groups include, but are not limited to, cyclohexanes, cyclohexenes, cyclopentanes, and cyclopentenes. Cycloalkyl groups may be substituted with alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Cycloalkyl groups can be fused to other cycloalkyl saturated or unsaturated, aryl, or heterocyclyl groups.

The term “dicarboxylic acid” as used herein refers to a group containing at least two carboxylic acid groups such as, e.g., saturated and unsaturated hydrocarbon dicarboxylic acids and salts thereof. Exemplary dicarboxylic acids include alkyl dicarboxylic acids. Dicarboxylic acids may be substituted with alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydrogen, hydroxyl, ketone, nitro, phosphate, sulfide, sulfinyl, sulfonyl, sulfonic acid, sulfonamide and thioketone. Dicarboxylic acids include, but are not limited to succinic acid, glutaric acid, adipic acid, suberic acid, sebacic acid, azelaic acid, maleic acid, phthalic acid, aspartic acid, glutamic acid, malonic acid, fumaric acid, (+)/(−)-malic acid, (+)/(−) tartaric acid, isophthalic acid, and terephthalic acid. Dicarboxylic acids further include carboxylic acid derivatives thereof, such as, e.g., anhydrides, imides, hydrazides (for example, succinic anhydride and succinimide).

The term “ester” refers to the structure —C(O)O—, —C(O)O—Rⱼ₋, —RₖC(O)O—Rⱼ₋, or —RₖC(O)O—, where 0 is not bound to hydrogen, and Rⱼ and Rₖ can independently be selected from alkoxy, aryloxy, alkyl, alkenyl, alkynyl, amide, amino, aryl, arylalkyl, cycloalkyl, ether, haloalkyl, heteroaryl, and heterocyclyl. Rₖ can be a hydrogen atom, but Rⱼ cannot be a hydrogen atom. The ester may be cyclic, for example the carbon atom and Rⱼ, the oxygen atom and Rₖ, or Rⱼ and Rₖ may be joined to form a 3- to 12-membered ring. Exemplary esters include, but are not limited to, alkyl esters wherein at least one of Rⱼ or Rₖ is alkyl, such as, e.g., —O—C(O)-alkyl, —C(O)—O-alkyl-, and -alkyl-C(O)—O— alkyl-. Exemplary esters also include aryl or heteoraryl esters, e.g. wherein at least one of Rⱼ or Rₖ is a heteroaryl group such as, e.g., pyridine, pyridazine, pyrimidine and pyrazine, such as, e.g., a nicotinate ester. Exemplary esters also include reverse esters having the structure —RₖC(O)O—, where the oxygen is bound to the parent molecule. Exemplary reverse esters include succinate, D-argininate, L-argininate, L-lysinate and D-lysinate. Esters also include carboxylic acid anhydrides and acid halides.

The terms “halo” or “halogen” as used herein refer to F, Cl, Br, or I.

The term “haloalkyl” as used herein refers to an alkyl group substituted with one or more halogen atoms. “Haloalkyls” also encompass alkenyl or alkynyl groups substituted with one or more halogen atoms.

The term “heteroaryl” as used herein refers to a mono-, bi-, or multi-cyclic, aromatic ring system containing one or more heteroatoms, for example 1-3 heteroatoms, such as, e.g., nitrogen, oxygen, and sulfur. Heteroaryls can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Heteroaryls can also be fused to non-aromatic rings. Illustrative examples of heteroaryl groups include, but are not limited to, pyridinyl, pyridazinyl, pyrimidyl, pyrazyl, triazinyl, pyrrolyl, pyrazolyl, imidazolyl, (1,2,3)- and (1,2,4)-triazolyl, pyrazinyl, pyrimidilyl, tetrazolyl, furyl, thienyl, isoxazolyl, thiazolyl, furyl, phenyl, isoxazolyl, and oxazolyl. Exemplary heteroaryl groups include, but are not limited to, a monocyclic aromatic ring, wherein the ring comprises 2-5 carbon atoms and 1-3 heteroatoms, referred to herein as “(C₂-C₅)heteroaryl.”

The terms “heterocycle,” “heterocyclyl,” or “heterocyclic” as used herein refer to a saturated or unsaturated 3-, 4-, 5-, 6- or 7-membered ring containing one, two, or three heteroatoms independently selected from nitrogen, oxygen, and sulfur. Heterocycles can be aromatic (heteroaryls) or non-aromatic. Heterocycles can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Heterocycles also include bicyclic, tricyclic, and tetracyclic groups in which any of the above heterocyclic rings is fused to one or two rings independently selected from aryls, cycloalkyls, and heterocycles. Exemplary heterocycles include acridinyl, benzimidazolyl, benzofuryl, benzothiazolyl, benzothienyl, benzoxazolyl, biotinyl, cinnolinyl, dihydrofuryl, dihydroindolyl, dihydropyranyl, dihydrothienyl, dithiazolyl, furyl, homopiperidinyl, imidazolidinyl, imidazolinyl, imidazolyl, indolyl, isoquinolyl, isothiazolidinyl, isothiazolyl, isoxazolidinyl, isoxazolyl, morpholinyl, oxadiazolyl, oxazolidinyl, oxazolyl, piperazinyl, piperidinyl, pyranyl, pyrazolidinyl, pyrazinyl, pyrazolyl, pyrazolinyl, pyridazinyl, pyridyl, pyrimidinyl, pyrimidyl, pyrrolidinyl, pyrrolidin-2-onyl, pyrrolinyl, pyrrolyl, quinolinyl, quinoxaloyl, tetrahydrofuryl, tetrahydroisoquinolyl, tetrahydropyranyl, tetrahydroquinolyl, tetrazolyl, thiadiazolyl, thiazolidinyl, thiazolyl, thienyl, thiomorpholinyl, thiopyranyl, and triazolyl.

The terms “hydroxy” and “hydroxyl” as used herein refer to —OH.

The term “hydroxyalkyl” as used herein refers to a hydroxy attached to an alkyl group.

The term “hydroxyaryl” as used herein refers to a hydroxy attached to an aryl group.

The term “ketone” as used herein refers to the structure —C(O)—Rn (such as, e.g., acetyl, —C(O)CH₃) or —Rₙ₋C(O)—Rₒ₋. The ketone can be attached to another group through Rₙ or Rₒ. Rₙ or Rₒ can be alkyl, alkenyl, alkynyl, cycloalkyl, heterocyclyl or aryl, or Rₙ or Rₒ can be joined to form a 3- to 12-membered ring.

The term “monoester” as used herein refers to an analogue of a dicarboxylic acid wherein one of the carboxylic acids is functionalized as an ester and the other carboxylic acid is a free carboxylic acid or salt of a carboxylic acid. Examples of monoesters include, but are not limited to, to monoesters of succinic acid, glutaric acid, adipic acid, suberic acid, sebacic acid, azelaic acid, oxalic and maleic acid.

The term “phenyl” as used herein refers to a 6-membered carbocyclic aromatic ring. The phenyl group can also be fused to a cyclohexane or cyclopentane ring. Phenyl can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, phosphate, sulfide, sulfinyl, sulfonyl, sulfonic acid, sulfonamide and thioketone.

The term “thioalkyl” as used herein refers to an alkyl group attached to a sulfur (—S— alkyl-).

“Alkyl,” “alkenyl,” “alkynyl”, “alkoxy”, “amino” and “amide” groups can be optionally substituted with or interrupted by or branched with at least one group selected from alkoxy, aryloxy, alkyl, alkenyl, alkynyl, amide, amino, aryl, arylalkyl, carbamate, carbonyl, carboxy, cyano, cycloalkyl, ester, ether, formyl, halogen, haloalkyl, heteroaryl, heterocyclyl, hydroxyl, ketone, phosphate, sulfide, sulfinyl, sulfonyl, sulfonic acid, sulfonamide, thioketone, ureido and N. The substituents may be branched to form a substituted or unsubstituted heterocycle or cycloalkyl.

As used herein, a suitable substitution on an optionally substituted substituent refers to a group that does not nullify the synthetic or pharmaceutical utility of the compounds of the present disclosure or the intermediates useful for preparing them. Examples of suitable substitutions include, but are not limited to: C₁₋₈ alkyl, alkenyl or alkynyl; C₁₋₆ aryl, C₂₋₅ heteroaryl; C₃₇ cycloalkyl; C₁₋₈ alkoxy; C₆ aryloxy; —CN; —OH; oxo; halo, carboxy; amino, such as, e.g., —NH(C₁₋₈ alkyl), —N(C₁₋₈ alkyl)₂, —NH((C₆)aryl), or —N((C₆)aryl)₂; formyl; ketones, such as, e.g., —CO(C₁₋₈ alkyl), —CO((C₆ aryl) esters, such as, e.g., —CO₂(C₁₋₈ alkyl) and —CO₂ (C₆ aryl). One of skill in art can readily choose a suitable substitution based on the stability and pharmacological and synthetic activity of the compound of the present disclosure.

The term “pharmaceutically acceptable carrier” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutically acceptable composition” as used herein refers to a composition comprising at least one compound as disclosed herein formulated together with one or more pharmaceutically acceptable carriers.

The term “pharmaceutically acceptable prodrugs” as used herein represents those prodrugs of the compounds of the present disclosure that are, within the scope of sound medical judgment, suitable for use in contact with the tissues of humans and lower animals without undue toxicity, irritation, allergic response, commensurate with a reasonable benefit/risk ratio, and effective for their intended use, as well as the zwitterionic forms, where possible, of the compounds of the present disclosure. A discussion is provided in Higuchi et al., “Prodrugs as Novel Delivery Systems,” ACS Symposium Series, Vol. 14, and in Roche, E. B., ed. Bioreversible Carriers in Drug Design, American Pharmaceutical Association and Pergamon Press, 1987, both of which are incorporated herein by reference.

The term “pharmaceutically acceptable salt(s)” refers to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfate, citrate, matate, acetate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds included in the present compositions, that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

The compounds of the disclosure may contain one or more chiral centers and/or double bonds and, therefore, exist as stereoisomers, such as, e.g., geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present disclosure encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly.

Individual stereoisomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well-known methods, such as, e.g., chiral-phase gas chromatography, chiral-phase high performance liquid chromatography, crystallizing the compound as a chiral salt complex, or crystallizing the compound in a chiral solvent. Stereoisomers can also be obtained from stereomerically-pure intermediates, reagents, and catalysts by well-known asymmetric synthetic methods.

Geometric isomers can also exist in the compounds of the present disclosure. The present disclosure encompasses the various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “E” are used in accordance with IUPAC standards. Unless otherwise specified, structures depicting double bonds encompass both the E and Z isomers.

Substituents around a carbon-carbon double bond alternatively can be referred to as “cis” or “trans,” where “cis” represents substituents on the same side of the double bond and “trans” represents substituents on opposite sides of the double bond. The arrangements of substituents around a carbocyclic ring are designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

The compounds disclosed herein may exist as tautomers and both tautomeric forms are intended to be encompassed by the scope of the present disclosure, even though only one tautomeric structure is depicted.

### EXEMPLARY EMBODIMENTS OF THE INVENTION

In some embodiments, the compound of Formula I is a compound according to Formula Ia:

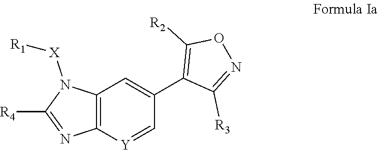

or a stereoisomer, tautomer, pharmaceutically acceptable salt, or hydrate thereof, wherein:
R₁ is selected from carbocycle (C₅-C₁₀) and heterocycle (C₂-C₁₀) optionally substituted with 1 to 5 groups independently selected from R₅;
R₂ is selected from hydrogen and alkyl (C₁-C₆) optionally substituted with halogen and hydroxyl;
R₃ is selected from alkyl (C₁-C₆) optionally substituted with halogen and hydroxyl, with the proviso that if R₂ and R₃ are methyls, then R₁ is different from:

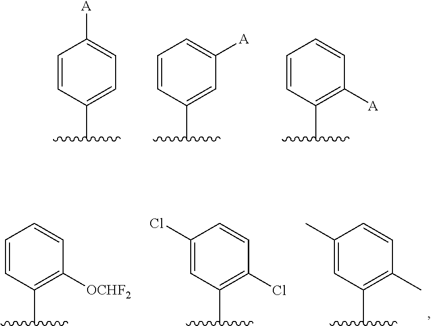

wherein A is selected from hydrogen, halogen, methoxy, —CN, —NO₂, —COOMe, and —CONMe₂;
R₄ is selected from hydrogen, alkyl (C₁-C₁₀), carbocycle (C₃-C₁₀), and heterocycle (C₂-C₁₀) optionally substituted with 1 to 5 groups independently selected from R₅;
each R₅ is independently selected from deuterium, alkyl(C₁-C₆) (such as, e.g., methyl, ethyl, propyl, isopropyl, butyl), alkoxy(C₁—C) (such as, e.g., methoxy, ethoxy, isopropoxy), amino (such as, e.g., —NH₂, —NHMe, —NHEt, —NHiPr, —NHBu —NMe₂, NMeEt, —NEt₂, —NEtBu), —NHC(O)NH-alkyl(C₁-C₆), halogen (such as, e.g., F, Cl), amide (such as, e.g., —NHC(O)Me, —NHC(O)Et, —C(O)NHMe, —C(O)NEt₂, —C(O)NiPr), —CF₃, —CN, —N₃, ketone (C₁-C₆) (such as, e.g., acetyl, —C(O)Et, —C(O)Pr), —S(O)-alkyl(C₁-C₄) (such as, e.g., —S(O)Me, —S(O)Et), —SO₂-alkyl(C₁-C₆) (such as, e.g., —SO₂Me, —SO₂Et, —SO₂Pr), thioalkyl(C₁-C₆) (such as, e.g., —SMe, —SEt, —SPr, —SBu), —COOH, and ester (such as, e.g., —C(O)OMe, —C(O)OEt, —C(O)OBu), each of which may be optionally substituted with hydrogen, F, Cl, Br, —OH, —NH₂, —NHMe, —OMe, —SMe, oxo, and/or thio-oxo;
X is selected from —CH₂— optionally substituted with 1 to 2 groups independently selected from R₅; and
Y is selected from N and CH.

In some embodiments, the compound of Formula I is a compound according to Formula Ib:

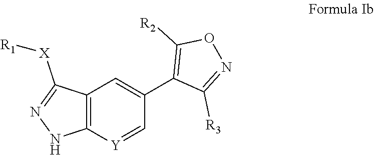

or a stereoisomer, tautomer, pharmaceutically acceptable salt, or hydrate thereof, wherein:
R₁ is selected from carbocycle (C₅-C₁₀) and heterocycle (C₂-C₁₀) optionally substituted with 1 to 5 groups independently selected from R₅;
R₂ is selected from hydrogen and alkyl (C₁-C₆) optionally substituted with halogen and hydroxyl;
R₃ is selected from alkyl (C₁-C₆) optionally substituted with halogen and hydroxyl, with the proviso that if R₂ and R₃ are methyls, then R₁ is different from:

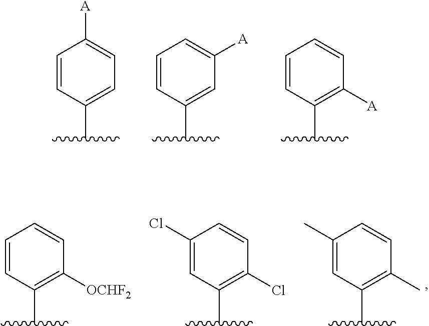

wherein A is selected from hydrogen, halogen, methoxy, —CN, —NO₂, —COOMe, and —CONMe₂;
each R₅ is independently selected from deuterium, alkyl(C₁-C₆) (such as, e.g., methyl, ethyl, propyl, isopropyl, butyl), alkoxy(C₁-C₆) (such as, e.g., methoxy, ethoxy, isopropoxy), amino (such as, e.g., —NH₂, —NHMe, —NHEt, —NHiPr, —NHBu —NMe₂, NMeEt, —NEt₂, —NEtBu), —NHC(O)NH-alkyl(C₁-C₆), halogen (such as, e.g., F, Cl), amide (such as, e.g., —NHC(O)Me, —NHC(O)Et, —C(O)NHMe, —C(O)NEt₂, —C(O)NiPr), —CF₃, —CN, —N₃, ketone (C₁-C₆) (such as, e.g., acetyl, —C(O)Et, —C(O)Pr), —S(O)-alkyl(C₁-C₄) (such as, e.g., —S(O)Me, —S(O)Et), —SO₂-alkyl(C₁-C₆) (such as, e.g., —SO₂Me, —SO₂Et, —SO₂Pr), thioalkyl(C₁-C₆) (such as, e.g., —SMe, —SEt, —SPr, —SBu), —COOH, and ester (such as, e.g., —C(O)OMe, —C(O)OEt, —C(O)OBu), each of which may be optionally substituted with hydrogen, F, Cl, Br, —OH, —NH₂, —NHMe, —OMe, —SMe, oxo, and/or thio-oxo;
X is selected from —CH₂— optionally substituted with 1 to 2 groups independently selected from R₅; and
Y is selected from N and CH.

In some embodiments, R₁ in the compound of Formula I, Formula Ia, or Formula Ib or a stereoisomer, tautomer, pharmaceutically acceptable salt, or hydrate thereof, is selected from carbocycle (C₅-C₁₀) optionally substituted with 1 to 5 groups independently selected from R₅; and W₁, W₂, W₃, Z₁, Z₂, R₂, R₃, R₄, R₅, X, and Y are as defined in any one or combination of paragraphs 83-118 herein.

In some embodiments, R₁ in the compound of Formula I, Formula Ia, or Formula Ib or a stereoisomer, tautomer, pharmaceutically acceptable salt, or hydrate thereof, is selected from phenyl groups optionally substituted with 1 to 5 groups independently selected from R₅; and W₁, W₂, W₃, Z₁, Z₂, R₂, R₃, R₄, R₅, X, and Y are as defined in any one or combination of paragraphs 83-118 herein.

In some embodiments, R₁ in the compound of Formula I, Formula Ia, or Formula Ib or a stereoisomer, tautomer, pharmaceutically acceptable salt, or hydrate thereof, is selected from phenyl groups substituted with 1 to 5 groups independently selected from thioalkyl(C₁-C₆) (such as, e.g., —SMe, —SEt, —SPr, —SBu), ester (such as, e.g., —C(O)OMe, —C(O)OEt, —C(O)OBu), —S(O)-alkyl(C₁-C₄) (such as, e.g., —S(O)Me, —S(O)Et), and —COOH; and W₁, W₂, W₃, Z₁, Z₂, R₂, R₃, R₄, R₅, X, and Y are as defined in any one or combination of paragraphs 83-118 herein.

In some embodiments, R₁ in the compound of Formula I, Formula Ia, or Formula Ib or a stereoisomer, tautomer, pharmaceutically acceptable salt, or hydrate thereof, is selected from phenyl groups substituted with 1 to 5 groups independently selected from —SMe, —C(O)OMe, —S(O)Me, and —COOH; and W₁, W₂, W₃, Z₁, Z₂, R₂, R₃, R₄, R₅, X, and Y are as defined in any one or combination of paragraphs 83-118 herein.

In some embodiments, R₁ in the compound of Formula I, Formula Ia, or Formula Ib or a stereoisomer, tautomer, pharmaceutically acceptable salt, or hydrate thereof, is unsubstituted phenyl; and W₁, W₂, W₃, Z₁, Z₂, R₂, R₃, R₄, R₅, X, and Y are as defined in any one or combination of paragraphs 83-118 herein.

In some embodiments, R₁ in the compound of Formula I, Formula Ia, or Formula Ib or a stereoisomer, tautomer, pharmaceutically acceptable salt, or hydrate thereof, is selected from heterocycles (C₂-C₀) optionally substituted with 1 to 5 groups independently selected from R₅; and W₁, W₂, W₃, Z₁, Z₂, R₂, R₃, R₄, R₅, X, and Y are as defined in any one or combination of paragraphs 83-118 herein.

In some embodiments, R₁ in the compound of Formula I, Formula Ia, or Formula Ib or a stereoisomer, tautomer, pharmaceutically acceptable salt, or hydrate thereof, is selected from:

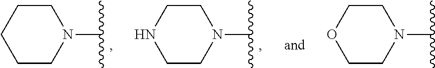 [SMILES: *N1CCCCC1 ; *N1CCNCC1 ; *N1CCOCC1]

optionally substituted with 1 to 5 groups independently selected from R₅; and W₁, W₂, W₃, Z₁, Z₂, R₂, R₃, R₄, R₅, X, and Y are as defined in any one or combination of paragraphs 83-118 herein.

In some embodiments, R₁ in the compound of Formula I, Formula Ia, or Formula Ib or a stereoisomer, tautomer, pharmaceutically acceptable salt, or hydrate thereof, is selected from:

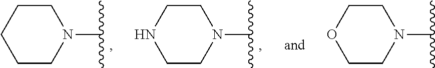 [SMILES: *N1CCCCC1 ; *N1CCNCC1 ; *N1CCOCC1]

optionally substituted with 1 to 5 groups independently selected from —CN, alkyl(C₁-C₆) (such as, e.g., methyl, ethyl, propyl, isopropyl, butyl), alkoxy(C₁-C₆) (such as, e.g., methoxy, ethoxy, isopropoxy), and halogen (such as, e.g., F, Cl); and W₁, W₂, W₃, Z₁, Z₂, R₂, R₃, R₄, R₅, X, and Y are as defined in any one or combination of paragraphs 83-118 herein.

In some embodiments, R₁ in the compound of Formula I, Formula Ia, or Formula Ib or a stereoisomer, tautomer, pharmaceutically acceptable salt, or hydrate thereof is selected from:

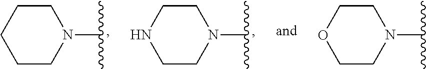

optionally substituted with 1 to 5 groups independently selected from —CN, methyl, methoxy, and F; and W₁, W₂, W₃, Z₁, Z₂, R₂, R₃, R₄, R₅, X, and Y are as defined in any one or combination of paragraphs 83-118 herein.

In some embodiments, R₁ in the compounds of Formula I, Formula Ia, or Formula Ib or a stereoisomer, tautomer, pharmaceutically acceptable salt, or hydrate thereof, is selected from:

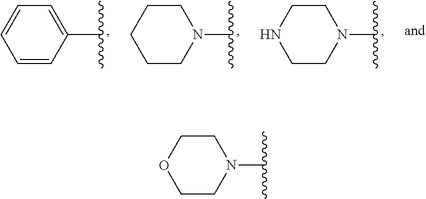

optionally substituted with 1 to 5 groups independently selected from R₅; and W₁, W₂, W₃, Z₁, Z₂, R₂, R₃, R₄, R₅, X, and Y are as defined in any one or combination of paragraphs 83-118 herein.

In some embodiments, R₁ in the compound of Formula I, Formula Ia, or Formula Ib or a stereoisomer, tautomer, pharmaceutically acceptable salt, or hydrate thereof, is selected from:

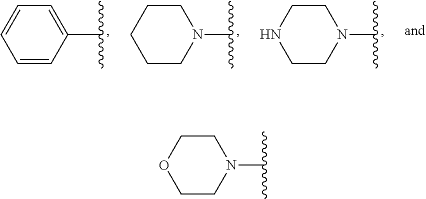

optionally substituted with 1 to 5 groups independently selected from thioalkyl(C₁-C₆) (such as, e.g., —SMe, —SEt, —SPr, —SBu), ester (such as, e.g., —C(O)OMe, —C(O)OEt, —C(O)OBu), —S(O)-alkyl(C₁-C₄) (such as, e.g., —S(O)Me, —S(O)Et), —COOH, —CN, alkyl(C₁-C₆) (such as, e.g., methyl, ethyl, propyl, isopropyl, butyl), alkoxy(C₁-C₆) (such as, e.g., methoxy, ethoxy, isopropoxy), and halogen (such as, e.g., F, Cl); and W₁, W₂, W₃, Z₁, Z₂, R₂, R₃, R₄, R₅, X, and Y are as defined in any one or combination of paragraphs 83-118 herein.

In some embodiments, R₁ in the compounds of Formula I, Formula Ia, or Formula Ib or a stereoisomer, tautomer, pharmaceutically acceptable salt, or hydrate thereof, is selected from:

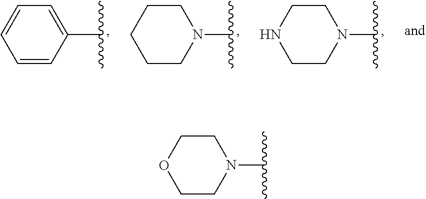

optionally substituted with 1 to 5 groups independently selected from —SMe, —C(O)OMe, —S(O)Me, —COOH, —CN, methyl, methoxy, and F; and W₁, W₂, W₃, Z₁, Z₂, R₂, R₃, R₄, R₅, X, and Y are as defined in any one or combination of paragraphs 83-118 herein.

In some embodiments, R₁ in the compound of Formula I, Formula Ia, or Formula Ib or a stereoisomer, tautomer, pharmaceutically acceptable salt, or hydrate thereof is selected from the following groups:

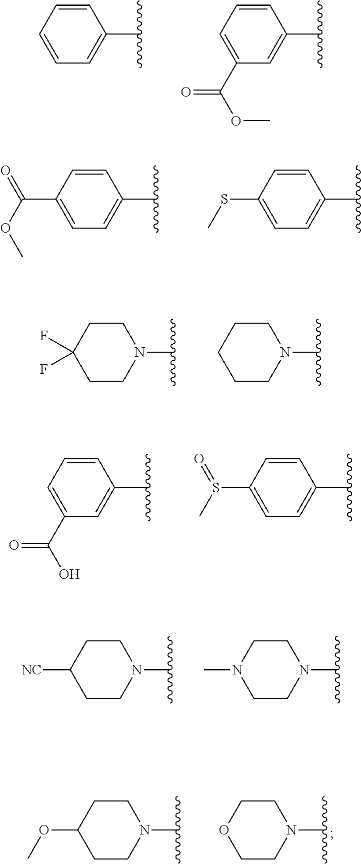

and W₁, W₂, W₃, Z₁, Z₂, R₂, R₃, R₄, R₅, X, and Y are as defined in any one or combination of paragraphs 83-118 herein.

In some embodiments, R₂ in the compound of Formula I, Formula Ia, or Formula Ib or a stereoisomer, tautomer, pharmaceutically acceptable salt, or hydrate thereof, is alkyl (C₁-C₆) optionally substituted with halogen and/or hydroxyl; and W₁, W₂, W₃, Z₁, Z₂, R₁, R₃, R₄, R₅, X, and Y are as defined in any one or combination of paragraphs 83-118 herein.

In some embodiments, R₂ in the compound of Formula I, Formula Ia, or Formula Ib or a stereoisomer, tautomer, pharmaceutically acceptable salt, or hydrate thereof, is a methyl group optionally substituted with halogen and/or hydroxyl; and W₁, W₂, W₃, Z₁, Z₂, R₁, R₃, R₄, R₅, X, and Y are as defined in any one or combination of paragraphs 83-118 herein.

In some embodiments, R₂ in the compound of Formula I, Formula Ia, or Formula Ib or a stereoisomer, tautomer, pharmaceutically acceptable salt, or hydrate thereof, is a methyl group; and W₁, W₂, W₃, Z₁, Z₂, R₁, R₃, R₄, R₅, X, and Y are as defined in any one or combination of paragraphs 83-118 herein.

In some embodiments, R₂ in the compound of Formula I, Formula Ia, or Formula Ib or a stereoisomer, tautomer, pharmaceutically acceptable salt, or hydrate thereof, is hydrogen; and W₁, W₂, W₃, Z₁, Z₂, R₁, R₃, R₄, R₅, X, and Y are as defined in any one or combination of paragraphs 83-118 herein.

In some embodiments, R₃ in the compound of Formula I, Formula Ia, or Formula Ib or a stereoisomer, tautomer, pharmaceutically acceptable salt, or hydrate thereof, is alkyl (C₁-C₆) optionally substituted with halogen and/or hydroxyl; and W₁, W₂, W₃, Z₁, Z₂, R₁, R₂, R₄, R₅, X, and Y are as defined in any one or combination of paragraphs 83-118 herein.

In some embodiments, R₃ in the compound of Formula I, Formula Ia, or Formula Ib or a stereoisomer, tautomer, pharmaceutically acceptable salt, or hydrate thereof, is a methyl group optionally substituted with halogen and/or hydroxyl; and W₁, W₂, W₃, Z₁, Z₂, R₁, R₂, R₄, R₅, X, and Y are as defined in any one or combination of paragraphs 83-118 herein.

In some embodiments, R₃ in the compound of Formula I, Formula Ia, or Formula Ib or a stereoisomer, tautomer, pharmaceutically acceptable salt, or hydrate thereof, is a methyl group; and W₁, W₂, W₃, Z₁, Z₂, R₁, R₂, R₄, R₅, X, and Y are as defined in any one or combination of paragraphs 83-118 herein.

In some embodiments, R₂ and R₃ in the compound of Formula I, Formula Ia, or Formula Ib or a stereoisomer, tautomer, pharmaceutically acceptable salt, or hydrate thereof, are methyl groups independently and optionally substituted with halogen and/or hydroxyl; and W₁, W₂, W₃, Z₁, Z₂, R₁, R₄, R₅, X, and Y are as defined in any one or combination of paragraphs 83-118 herein.

In some embodiments, R₂ and R₃ in the compound of Formula I, Formula Ia, or Formula Ib or a stereoisomer, tautomer, pharmaceutically acceptable salt, or hydrate thereof, are methyl groups; and W₁, W₂, W₃, Z₁, Z₂, R₁, R₄, R₅, X, and Y are as defined in any one or combination of paragraphs 83-118 herein.

In some embodiments, R₂ in the compound of Formula I, Formula Ia, or Formula Ib or a stereoisomer, tautomer, pharmaceutically acceptable salt, or hydrate thereof, is hydrogen, and R₃ is selected from alkyl (C₁-C₆) optionally substituted with halogen and/or hydroxyl; and W₁, W₂, W₃, Z₁, Z₂, R₁, R₄, R₅, X, and Y are as defined in any one or combination of paragraphs 83-118 herein.

In some embodiments, R₄ in the compound of Formula I or Formula Ia or a stereoisomer, tautomer, pharmaceutically acceptable salt, or hydrate thereof, is hydrogen; and W₁, W₂, W₃, Z₁, Z₂, R₁, R₂, R₃, R₅, X, and Y are as defined in any one or combination of paragraphs 83-118 herein.

In some embodiments, R₄ in the compound of Formula I or Formula Ia or a stereoisomer, tautomer, pharmaceutically acceptable salt, or hydrate thereof, is selected from alkyl (C₁-C₆) optionally substituted with 1 to 5 groups independently selected from R₅; and W₁, W₂, W₃, Z₁, Z₂, R₁, R₂, R₃, R₅, X, and Y are as defined in any one or combination of paragraphs 83-118 herein.

In some embodiments, R₄ in the compound of Formula I or Formula Ia or a stereoisomer, tautomer, pharmaceutically acceptable salt, or hydrate thereof, is selected from methyl optionally substituted with 1 to 5 groups independently selected from R₅; and W₁, W₂, W₃, Z₁, Z₂, R₁, R₂, R₃, R₅, X, and Y are as defined in any one or combination of paragraphs 83-118 herein.

In some embodiments, R₄ in the compound of Formula I or Formula Ia or a stereoisomer, tautomer, pharmaceutically acceptable salt, or hydrate thereof, is methyl; and W₁, W₂, W₃, Z₁, Z₂, R₁, R₂, R₃, R₅, X, and Y are as defined in any one or combination of paragraphs 83-118 herein.

In some embodiments, R₄ in the compound of Formula I or Formula Ia or a stereoisomer, tautomer, pharmaceutically acceptable salt, or hydrate thereof, is selected from heterocycle (C₂-C₆) optionally substituted with 1 to 5 groups independently selected from R₅; and W₁, W₂, W₃, Z₁, Z₂, R₁, R₂, R₃, R₅, X, and Y are as defined in any one or combination of paragraphs 83-118 herein.

In some embodiments, R₄ in the compound of Formula I or Formula Ia or a stereoisomer, tautomer, pharmaceutically acceptable salt, or hydrate thereof, is selected from carbocycle (C₃-C₁₀) optionally substituted with 1 to 5 groups independently selected from R₅; and W₁, W₂, W₃, Z₁, Z₂, R₁, R₂, R₃, R₅, X, and Y are as defined in any one or combination of paragraphs 83-118 herein.

In some embodiments, each R₅ in the compound of Formula I, Formula Ia, or Formula Ib or stereoisomer, tautomer, pharmaceutically acceptable salt, or hydrate thereof, is independently selected from deuterium, alkyl(C₁-C₆) (such as, e.g., methyl, ethyl, propyl, isopropyl, butyl), alkoxy(C₁-C₆) (such as, e.g., methoxy, ethoxy, isopropoxy), amino (such as, e.g., —NH₂, —NHMe, —NHEt, —NHiPr, —NHBu —NMe₂, NMeEt, —NEt₂, —NEtBu), —NHC(O)NH-alkyl(C₁-C₆), halogen (such as, e.g., F, Cl), amide (such as, e.g., —NHC(O)Me, —NHC(O)Et, —C(O)NHMe, —C(O)NEt₂, —C(O)NiPr), —CF₃, —CN, —N₃, ketone (C₁-C₆) (such as, e.g., acetyl, —C(O)Et, —C(O)Pr), —S(O)-alkyl(C₁-C₄) (such as, e.g., —S(O)Me, —S(O)Et), —SO2-alkyl(C₁-C₆) (such as, e.g., —SO₂Me, —SO₂Et, —SO₂Pr), and thioalkyl(C₁-C₆) (such as, e.g., —SMe, —SEt, —SPr, —SBu); and W₁, W₂, W₃, Z₁, Z₂, R₁, R₂, R₃, R₄, X, and Y are as defined in any one or combination of paragraphs 83-118 herein.

In some embodiments, each R₅ in the compound of Formula I, Formula Ia, or Formula Ib or a stereoisomer, tautomer, pharmaceutically acceptable salt, or hydrate thereof, is independently selected from thioalkyl(C₁-C₆) (such as, e.g., —SMe, —SEt, —SPr, —SBu), ester (such as, e.g., —C(O)OMe, —C(O)OEt, —C(O)OBu), —S(O)-alkyl(C₁-C₄) (such as, e.g., —S(O)Me, —S(O)Et), —COOH, —CN, alkyl(C₁-C₆) (such as, e.g., methyl, ethyl, propyl, isopropyl, butyl), alkoxy(C₁-C₆) (such as, e.g., methoxy, ethoxy, isopropoxy), and halogen (such as, e.g., F, Cl); and W₁, W₂, W₃, Z₁, Z₂, R₁, R₂, R₃, R₄, X, and Y are as defined in any one or combination of paragraphs 83-118 herein.

In some embodiment, Y is CH in the compound of Formula I, Formula Ia, or Formula Ib or a stereoisomer, tautomer, pharmaceutically acceptable salt, or hydrate thereof, and W₁, W₂, W₃, Z₁, Z₂, R₁, R₂, R₃, R₄, R₅, and X are defined in any one or combination of paragraphs 83-118 herein.

In some embodiments, Y is N in the compound of Formula I, Formula Ia, or Formula Ib or stereoisomer, tautomer, pharmaceutically acceptable salt, or hydrate thereof, and W₁, W₂, W₃, Z₁, Z₂, R₁, R₂, R₃, R₄, R₅, and X are as defined in any one or combination of paragraphs 83-118 herein.

In some embodiments, X is —CH₂— in the compound of Formula I, Formula Ia, or Formula Ib or stereoisomer, tautomer, pharmaceutically acceptable salt, or hydrate thereof, and W₁, W₂, W₃, Z₁, Z₂, R₁, R₂, R₃, R₄, R₅, and Y are as defined in any one or combination of paragraphs 83-118 herein.

In some embodiments, the compound of Formula I or Formula Ia is selected from:
3,5-Dimethyl-4-(2-methyl-1-(4-(methylthio)benzyl)-1H-imidazo[4,5-b]pyridin-6-yl)isoxazole (Example 1);
Methyl 3-((6-(3,5-dimethylisoxazol-4-yl)-2-methyl-1H-imidazo[4,5-b]pyridin-1-yl)methyl)benzoate (Example 2);
Methyl 4-((6-(3,5-dimethylisoxazol-4-yl)-2-methyl-1H-imidazo[4,5-b]pyridin-1-yl)methyl)benzoate (Example 3);
3,5-Dimethyl-4-(2-methyl-1-(3-(methylthio)benzyl)-1H-imidazo[4,5-b]pyridin-6-yl)isoxazole (Example 4);
3,5-Dimethyl-4-(2-methyl-1-(4-(methylsulfinyl)benzyl)-1H-imidazo[4,5-b]pyridin-6-yl)isoxazole (Example 5);
3-((6-(3,5-Dimethylisoxazol-4-yl)-2-methyl-1H-imidazo[4,5-b]pyridin-1-yl)methyl)benzoic acid (Example 6);
(4-(1-Benzyl-2-methyl-1H-imidazo[4,5-b]pyridin-6-yl)-3-methylisoxazol-5-yl)methanol (Example 7);
4-(1-Benzyl-2-methyl-1H-imidazo[4,5-b]pyridin-6-yl)-3-methylisoxazole (Example 8);
   and stereoisomers, tautomers, pharmaceutically acceptable salts, or hydrates thereof.

In some embodiments, the compound of Formula I or Formula Ib is selected from:
4-(3-((4,4-Difluoropiperidin-1-yl)methy)-1H-indazol-5-yl)-3, methylisoxazole (Example 9);
3,5-Dimethyl-4-[3-(1-piperidylmethyl)-1H-indazol-5-yl]isoxazole (Example 10);
3,5-Dimethyl-4-[3-[(4-methylpiperazin-1-yl)methyl]-1H-indazol-5-yl]isoxazole (Example 11);
1-[[5-(3,5-Dimethylisoxazol-4-yl)-1H-indazol-3-yl]methyl]piperidine-4-carbonitrile (Example 12);
4-[3-[(4-Methoxy-1-piperidyl)methyl]-1H-indazol-5-yl]-3,5-dimethyl-isoxazole (Example 13);
4-[[5-(3,5-Dimethylisoxazol-4-yl)-1H-indazol-3-yl]methyl]morpholine (Example 14);
   and stereoisomers, tautomers, pharmaceutically acceptable salts, or hydrates thereof.

Another aspect of the invention provides a method for inhibition of BET protein function by binding to bromodomains, and their use in the treatment and prevention of diseases and conditions in a mammal (e.g., a human) comprising administering a therapeutically effective amount of a compound of Formula I, Formula Ia, or Formula Ib, or a stereoisomer, tautomer, pharmaceutically acceptable salt, or hydrates thereof.

In one embodiment, because of potent effects of BET inhibitors in vitro on IL-6 and IL-17 transcription, BET inhibitor compounds of Formula I, Formula Ia, Formula Ib, or stereoisomers, tautomers, pharmaceutically acceptable salts, and hydrates thereof may be used as therapeutics for inflammatory disorders in which IL-6 and/or IL-17 have been implicated in disease. The following autoimmune diseases are amenable to therapeutic use of BET inhibition by administration of a compound of Formula I, Formula Ia, or Formula Ib or a stereoisomer, tautomer, pharmaceutically acceptable salt, or hydrate thereof because of a prominent role of IL-6 and/or IL-17: Acute Disseminated Encephalomyelitis (T. Ishizu et al., “CSF cytokine and chemokine profiles in acute disseminated encephalomyelitis,” J Neuroimmunol 175(1-2): 52-8 (2006)), Agammaglobulinemia (M. Gonzalez-Serrano, et al.,” Increased Pro-inflammatory Cytokine Production After Lipopolysaccharide Stimulation in Patients with X-linked Agammaglobulinemia,” J Clin Immunol 32(5):967-74 (2012)), Allergic Disease (L. McKinley et al., “TH17 cells mediate steroid-resistant airway inflammation and airway hyperresponsiveness in mice,” J Immunol 181(6):4089-97 (2008)), Ankylosing spondylitis (A. Taylan et al., “Evaluation of the T helper 17 axis in ankylosing spondylitis,” Rheumatol Int 32(8):2511-5 (2012)), Anti-GBM/Anti-TBM nephritis (Y. Ito et al., “Pathogenic significance of interleukin-6 in a patient with antiglomerular basement membrane antibody-induced glomerulonephritis with multinucleated giant cells,” Am J Kidney Dis 26(1):72-9 (1995)), Anti-phospholipid syndrome (P. Soltesz et al., “Immunological features of primary anti-phospholipid syndrome in connection with endothelial dysfunction,” Rheumatology (Oxford) 47(11):1628-34 (2008)), Autoimmune aplastic anemia (Y. Gu et al., “Interleukin (IL)-17 promotes macrophages to produce IL-8, IL-6 and tumour necrosis factor-alpha in aplastic anaemia,” Br J Haematol 142(1):109-14 (2008)), Autoimmune hepatitis (L Zhao et al., “Interleukin-17 contributes to the pathogenesis of autoimmune hepatitis through inducing hepatic interleukin-6 expression,” PLoS One 6(4):e18909 (2011)), Autoimmune inner ear disease (B. Gloddek et al., “Pharmacological influence on inner ear endothelial cells in relation to the pathogenesis of sensorineural hearing loss,” Adv Otorhinolaryngol 59:75-83 (2002)), Autoimmune myocarditis (T. Yamashita et al., “IL-6-mediated Th17 differentiation through RORgammat is essential for the initiation of experimental autoimmune myocarditis,” Cardiovasc Res 91(4):640-8 (2011)), Autoimmune pancreatitis (J. Ni et al., “Involvement of Interleukin-17A in Pancreatic Damage in Rat Experimental Acute Necrotizing Pancreatitis,” Inflammation (2012)), Autoimmune retinopathy (5. Hohki et al., “Blockade of interleukin-6 signaling suppresses experimental autoimmune uveoretinitis by the inhibition of inflammatory Th17 responses,” Exp Eye Res 91(2):162-70 (2010)), Autoimmune thrombocytopenic purpura (D. Ma et al., “Profile of Th17 cytokines (IL-17, TGF-beta, IL-6) and Th1 cytokine (IFN-gamma) in patients with immune thrombocytopenic purpura,” Ann Hematol 87(11):899-904 (2008)), Behcet's Disease (T. Yoshimura et al., “Involvement of Th17 cells and the effect of anti-IL-6 therapy in autoimmune uveitis,” Rheumatology (Oxford) 48(4):347-54 (2009)), Bullous pemphigoid (L. D'Auria et al., “Cytokines and bullous pemphigoid,” Eur Cytokine Netw 10(2):123-34 (1999)), Castleman's Disease (H. El-Osta and R. Kurzrock, “Castleman's disease: from basic mechanisms to molecular therapeutics,” Oncologist 16(4):497-511 (2011)), Celiac Disease (A. Lahdenpera et al., “Up-regulation of small intestinal interleukin-17 immunity in untreated coeliac disease but not in potential coeliac disease or in type 1 diabetes,” Clin Exp Immunol 167(2):226-34 (2012)), Churg-Strauss syndrome (A. Fujioka et al., “The analysis of mRNA expression of cytokines from skin lesions in Churg-Strauss syndrome,” J Dermatol 25(3):171-7 (1998)), Crohn's Disease (V. Holtta et al., “IL-23/IL-17 immunity as a hallmark of Crohn's disease,” Inflamm Bowel Dis 14(9):1175-84 (2008)), Cogan's syndrome (M. Shibuya et al., “Successful treatment with tocilizumab in a case of Cogan's syndrome complicated with aortitis,” Mod Rheumatol (2012)), Dry eye syndrome (C. De Paiva et al., “IL-17 disrupts corneal barrier following desiccating stress,” Mucosal Immunol 2(3):243-53 (2009)), Essential mixed cryoglobulinemia (A. Antonelli et al., “Serum levels of proinflammatory cytokines interleukin-1 beta, interleukin-6, and tumor necrosis factor alpha in mixed cryoglobulinemia,” Arthritis Rheum 60(12):3841-7 (2009)), Dermatomyositis (G. Chevrel et al., “Interleukin-17 increases the effects of IL-1 beta on muscle cells: arguments for the role of T cells in the pathogenesis of myositis,” J Neuroimmunol 137(1-2):125-33 (2003)), Devic's Disease (U. Linhares et al., “The Ex Vivo Production of IL-6 and IL-21 by CD4(+) T Cells is Directly Associated with Neurological Disability in Neuromyelitis Optica Patients,” J Clin Immunol (2012)), Encephalitis (D. Kyburz and M. Corr, “Th17 cells generated in the absence of TGF-beta induce experimental allergic encephalitis upon adoptive transfer,” Expert Rev Clin Immunol 7(3):283-5 (2011)), Eosinophlic esophagitis (P. Dias and G. Banerjee, “The Role of Th17/IL-17 on Eosinophilic Inflammation,” J Autoimmun (2012)), Eosinophilic fasciitis (P. Dias and G. Banerjee, J Autoimmun (2012)), Erythema nodosum (I. Kahawita and D. Lockwood, “Towards understanding the pathology of erythema nodosum leprosum,” Trans R Soc Trop Med Hyg 102(4):329-37 (2008)), Giant cell arteritis (J. Deng et al., “Th17 and Th1 T-cell responses in giant cell arteritis,” Circulation 121(7):906-15 (2010)), Glomerulonephritis (J. Ooi et al., “Review: T helper 17 cells: their role in glomerulonephritis,” Nephrology (Carlton) 15(5):513-21 (2010)), Goodpasture's syndrome (Y. Ito et al., “Pathogenic significance of interleukin-6 in a patient with antiglomerular basement membrane antibody-induced glomerulonephritis with multinucleated giant cells,” Am J Kidney Dis 26(1):72-9 (1995)), Granulomatosis with Polyangitis (Wegener's) (H. Nakahama et al., “Distinct responses of interleukin-6 and other laboratory parameters to treatment in a patient with Wegener's granulomatosis,” Intern Med 32(2):189-92 (1993)), Graves' Disease (S. Kim et al., “Increased serum interleukin-17 in Graves' ophthalmopathy,” Graefes Arch Clin Exp Ophthalmol 250(10):1521-6 (2012)), Guillain-Barre syndrome (M. Lu and J. Zhu, “The role of cytokines in Guillain-Barre syndrome,” J Neurol 258(4):533-48 (2011)), Hashimoto's thyroiditis (N. Figueroa-Vega et al., “Increased circulating pro-inflammatory cytokines and Th17 lymphocytes in Hashimoto's thyroiditis,” J Clin Endocrinol Metab 95(2):953-62 (2009)), Hemolytic anemia (L. Xu et al., “Critical role of Th17 cells in development of autoimmune hemolytic anemia,” Exp Hematol (2012)), Henoch-Schonlein purpura (H. Jen et al., “Increased serum interleukin-17 and peripheral Th17 cells in children with acute Henoch-Schonlein purpura,” Pediatr Allergy Immunol 22(8):862-8 (2011)), IgA nephropathy (F. Lin et al., “Imbalance of regulatory T cells to Th17 cells in IgA nephropathy,” Scand J Clin Lab Invest 72(3):221-9 (2012)), Inclusion body myositis (P. Baron et al., “Production of IL-6 by human myoblasts stimulated with Abeta: relevance in the pathogenesis of IBM,” Neurology 57(9):1561-5 (2001)), Type I diabetes (A. Belkina and G. Denis, Nat Rev Cancer 12(7):465-77 (2012)), Interstitial cystitis (L Lamale et al., “Interleukin-6, histamine, and methylhistamine as diagnostic markers for interstitial cystitis,” Urology 68(4):702-6 (2006)), Kawasaki's Disease (5. Jia et al., “The T helper type 17/regulatory T cell imbalance in patients with acute Kawasaki disease,” Clin Exp Immunol 162(1):131-7 (2010)), Leukocytoclastic vasculitis (Min, C. K., et al., “Cutaneous leucoclastic vasculitis (LV) following bortezomib therapy in a myeloma patient; association with pro-inflammatory cytokines,” Eur J Haematol 76(3):265-8 (2006)), Uchen planus (N. Rhodus et al., “Proinflammatory cytokine levels in saliva before and after treatment of (erosive) oral lichen planus with dexamethasone,” Oral Dis 12(2):112-6 (2006)), Lupus (SLE) (M. Mok et al., “The relation of interleukin 17 (IL-17) and IL-23 to Th1/Th2 cytokines and disease activity in systemic lupus erythematosus,” J Rheumatol 37(10):2046-52 (2010)), Microscopic polyangitis (A. Muller Kobold et al., “In vitro up-regulation of E-selectin and induction of interleukin-6 in endothelial cells by autoantibodies in Wegener's granulomatosis and microscopic polyangiitis,” Clin Exp Rheumatol 17(4):433-40 (1999)), Multiple sclerosis (F. Jadidi-Niaragh and A. Mirshafiey, “Th17 cell, the new player of neuroinflammatory process in multiple sclerosis,” Scand J Immunol 74(1):1-13 (2011)), Myasthenia gravis (R. Aricha et al., “Blocking of IL-6 suppresses experimental autoimmune myasthenia gravis,” J Autoimmun 36(2):135-41 (2011)), myositis (G. Chevrel et al., “Interleukin-17 increases the effects of IL-1 beta on muscle cells: arguments for the role of T cells in the pathogenesis of myositis,” J Neuroimmunol 137(1-2):125-33 (2003)), Optic neuritis (S. Icoz et al., “Enhanced IL-6 production in aquaporin-4 antibody positive neuromyelitis optica patients,” Int J Neurosci 120(1):71-5 (2010)), Pemphigus (E. Lopez-Robles et al., “TNFalpha and IL-6 are mediators in the blistering process of pemphigus,” Int J Dermatol 40(3):185-8 (2001)), POEMS syndrome (K. Kallen et al., “New developments in IL-6 dependent biology and therapy: where do we stand and what are the options?” Expert Opin Investig Drugs 8(9):1327-49 (1999)), Polyarteritis nodosa (T. Kawakami et al., “Serum levels of interleukin-6 in patients with cutaneous polyarteritis nodosa,” Acta Derm Venereal 92(3):322-3 (2012)), Primary biliary cirrhosis (K. Harada et al., “Periductal interleukin-17 production in association with biliary innate immunity contributes to the pathogenesis of cholangiopathy in primary biliary cirrhosis,” Clin Exp Immunol 157(2):261-70 (2009)), Psoriasis (S. Fujishima et al., “Involvement of IL-17F via the induction of IL-6 in psoriasis,” Arch Dermatol Res 302(7):499-505 (2010)), Psoriatic arthritis (S. Raychaudhuri et al., IL-17 receptor and its functional significance in psoriatic arthritis,” Mol Cell Biochem 359(1-2):419-29 (2012)), Pyoderma gangrenosum (T. Kawakami et al., “Reduction of interleukin-6, interleukin-8, and anti-phosphatidylserine-prothrombin complex antibody by granulocyte and monocyte adsorption apheresis in a patient with pyoderma gangrenosum and ulcerative colitis,” Am J Gastroenterol 104(9):2363-4 (2009)), Relapsing polychondritis (M. Kawai et al., “Sustained response to tocilizumab, anti-interleukin-6 receptor antibody, in two patients with refractory relapsing polychondritis,” Rheumatology (Oxford) 48(3):318-9 (2009)), Rheumatoid arthritis (Z. Ash and P. Emery, “The role of tocilizumab in the management of rheumatoid arthritis,” Expert Opin Biol Ther, 12(9):1277-89 (2012)), Sarcoidosis (F. Belli et al., “Cytokines assay in peripheral blood and bronchoalveolar lavage in the diagnosis and staging of pulmonary granulomatous diseases,” Int J Immunopathol Pharmacol 13(2):61-67 (2000)), Scleroderma (T. Radstake et al., “The pronounced Th17 profile in systemic sclerosis (SSc) together with intracellular expression of TGFbeta and IFNgamma distinguishes SSc phenotypes,” PLoS One, 4(6): e5903 (2009)), Sjogren's syndrome (G. Katsifis et al., “Systemic and local interleukin-17 and linked cytokines associated with Sjogren's syndrome immunopathogenesis,”Am J Pathol 175(3):1167-77 (2009)), Takayasu's arteritis (Y. Sun et al., “MMP-9 and IL-6 are potential biomarkers for disease activity in Takayasu's arteritis,” Int J Cardiol 156(2):236-8 (2012)), Transverse myelitis (J. Graber et al., “Interleukin-17 in transverse myelitis and multiple sclerosis,” J Neuroimmunol 196(1-2):124-32 (2008)), Ulcerative colitis (J. Mudter and M. Neurath, “11-6 signaling in inflammatory bowel disease: pathophysiological role and clinical relevance,” Inflamm Bowel Dis 13(8):1016-23 (2007)), Uveitis (H. Haruta et al., “Blockade of interleukin-6 signaling suppresses not only th17 but also interphotoreceptor retinoid binding protein-specific Th1 by promoting regulatory T cells in experimental autoimmune uveoretinitis,” Invest Ophthalmol Vis Sci 52(6):3264-71 (2011)), and Vitiligo (D. Bassiouny and O. Shaker, “Role of interleukin-17 in the pathogenesis of vitiligo,” Clin Exp Dermatol 36(3):292-7 115. (2011)). Thus, the invention includes compounds of Formula I (including Formula Ia and Formula Ib), stereoisomers, tautomers, pharmaceutically acceptable salts, or hydrates thereof; pharmaceutical compositions comprising one or more of those compounds; and methods of using those compounds or compositions for treating these diseases.

Acute and chronic (non-autoimmune) inflammatory diseases characterized by increased expression of pro-inflammatory cytokines, including IL-6, MCP-1, and IL-17, would also be amenable to therapeutic BET inhibition. These include, but are not limited to, sinusitis (D. Bradley and S. Kountakis, “Role of interleukins and transforming growth factor-beta in chronic rhinosinusitis and nasal polyposis,” Laryngoscope 115(4):684-6 (2005)), pneumonitis (Besnard, A. G., et al., “Inflammasome-IL-1-Th17 response in allergic lung inflammation” J Mol Cell Biol 4(1):3-10 (2012)), osteomyelitis (T. Yoshii et al., “Local levels of interleukin-1beta, -4, -6 and tumor necrosis factor alpha in an experimental model of murine osteomyelitis due to staphylococcus aureus,” Cytokine 19(2):59-65 2002), gastritis (T. Bayraktaroglu et al., “Serum levels of tumor necrosis factor-alpha, interleukin-6 and interleukin-8 are not increased in dyspeptic patients with Helicobacter pylori-associated gastritis,” Mediators Inflamm 13(1):25-8 (2004)), enteritis (K. Mitsuyama et al., “STAT3 activation via interleukin 6 trans-signalling contributes to ileitis in SAMP1/Yit mice,” Gut 55(9):1263-9. (2006)), gingivitis (R. Johnson et al., “Interleukin-11 and IL-17 and the pathogenesis of periodontal disease,” J Periodontol 75(1):37-43 (2004)), appendicitis (S. Latifi et al., “Persistent elevation of serum interleukin-6 in intraabdominal sepsis identifies those with prolonged length of stay,” J Pediatr Surg 39(10):1548-52 (2004)), irritable bowel syndrome (M. Ortiz-Lucas et al., “Irritable bowel syndrome immune hypothesis. Part two: the role of cytokines,” Rev Esp Enferm Dig 102(12):711-7 (2010)), tissue graft rejection (L Kappel et al., “IL-17 contributes to CD4-mediated graft-versus-host disease,” Blood 113(4):945-52 (2009)), chronic obstructive pulmonary disease (COPD) (S. Traves and L. Donnelly, “Th17 cells in airway diseases,” Curr Mol Med 8(5):416-26 (2008)), septic shock (toxic shock syndrome, SIRS, bacterial sepsis, etc) (E. Nicodeme et al., Nature 468(7327):1119-23 (2010)), osteoarthritis (L Chen et al., “IL-17RA aptamer-mediated repression of IL-6 inhibits synovium inflammation in a murine model of osteoarthritis,” Osteoarthritis Cartilage 19(6):711-8 (2011)), acute gout (W. Urano et al., “The inflammatory process in the mechanism of decreased serum uric acid concentrations during acute gouty arthritis,” J Rheumatol 29(9):1950-3 (2002)), acute lung injury (S. Traves and L. Donnelly, “Th17 cells in airway diseases,” Curr Mol Med 8(5):416-26 (2008)), acute renal failure (E. Simmons et al., “Plasma cytokine levels predict mortality in patients with acute renal failure,” Kidney Int 65(4):1357-65 (2004)), burns (P. Paquet and G. Pierard, “Interleukin-6 and the skin,” Int Arch Allergy Immunol 109(4):308-17 (1996)), Herxheimer reaction (G. Kaplanski et al., “Jarisch-Herxheimer reaction complicating the treatment of chronic Q fever endocarditis: elevated TNFalpha and IL-6 serum levels,” J Infect 37(1):83-4 (1998)), and SIRS associated with viral infections (A. Belkinaand G. Denis, Nat Rev Cancer 12(7):465-77 (2012)). Thus, the invention includes compounds of Formula I (including Formula Ia and Formula Ib), stereoisomers, tautomers, pharmaceutically acceptable salts, or hydrates thereof; pharmaceutical compositions comprising one or more of those compounds; and methods of using those compounds or compositions for treating these diseases.

In one embodiment, BET inhibitor compounds of Formula I, Formula Ia, or Formula Ib, or stereoisomers, tautomers, pharmaceutically acceptable salts, or hydrates thereof, or compositions comprising one or more of those compounds may be used for treating rheumatoid arthritis (RA) and multiple sclerosis (MS). Strong proprietary data exist for the utility of BET inhibitors in preclinical models of RA and MS. R. Jahagirdar et al., “An Orally Bioavailable Small Molecule RVX-297 Significantly Decreases Disease in a Mouse Model of Multiple Sclerosis,” World Congress of Inflammation, Paris, France (2011). Both RA and MS are characterized by a dysregulation of the IL-6 and IL-17 inflammatory pathways (A. Kimura and T. Kishimoto, “IL-6: regulator of Treg/Th17 balance,” Eur J Immunol 40(7):1830-5 (2010)) and thus would be especially sensitive to BET inhibition. In another embodiment, BET inhibitor compounds of Formula I, Formula Ia, or Formula Ib, or stereoisomers, tautomers, pharmaceutically acceptable salts, or hydrates thereof, or compositions comprising one or more of those compounds, may be used for treating sepsis and associated afflictions. BET inhibition has been shown to inhibit development of sepsis, in part, by inhibiting IL-6 expression, in preclinical models in both published (E. Nicodeme et al., Nature 468(7327):1119-23 (2010)) and proprietary data.

In one embodiment, BET inhibitor compound of Formula I, Formula Ia, or Formula Ib, or stereoisomers, tautomers, pharmaceutically acceptable salts, or hydrates thereof, or compositions comprising one or more of those compounds may be used to treat cancer. Cancers that have an overexpression, translocation, amplification, or rearrangement c-myc or other myc family oncoproteins (MYCN, L-myc) are particularly sensitive to BET inhibition. J. Delmore et al., Cell 146(6):904-17 (2010); J. Mertz et al., Proc Natl Acad Sci USA 108(40):16669-74 (2011). These cancers include, but are not limited to, B-acute lymphocytic leukemia, Burkitt's lymphoma, Diffuse large cell lymphoma, Multiple myeloma, Primary plasma cell leukemia, Atypical carcinoid lung cancer, Bladder cancer, Breast cancer, Cervix cancer, Colon cancer, Gastric cancer, Glioblastoma, Hepatocellular carcinoma, Large cell neuroendocrine carcinoma, Medulloblastoma, Melanoma, nodular, Melanoma, superficial spreading, Neuroblastoma, esophageal squamous cell carcinoma, Osteosarcoma, Ovarian cancer, Prostate cancer, Renal clear cell carcinoma, Retinoblastoma, Rhabdomyosarcoma, and Small cell lung carcinoma. M. Vita and M. Henriksson, Semin Cancer Biol 16(4):318-30 (2006).

In one embodiment, BET inhibitor compounds of Formula I, Formula Ia, or Formula Ib, or stereoisomers, tautomers, pharmaceutically acceptable salts, or hydrates thereof, or compositions comprising one or more of those compounds may be used to treat cancers that result from an aberrant regulation (overexpression, translocation, etc) of BET proteins. These include, but are not limited to, NUT midline carcinoma (Brd3 or Brd4 translocation to nutlin 1 gene) (C. French Cancer Genet Cytogenet 203(1):16-20 (2010)), B-cell lymphoma (Brd2 overexpression) (R. Greenwald et al., Blood 103(4):1475-84 (2004)), non-small cell lung cancer (BrdT overexpression) (C. Grunwald et al., “Expression of multiple epigenetically regulated cancer/germline genes in nonsmall cell lung cancer,” Int J Cancer 118(10):2522-8 (2006)), esophageal cancer and head and neck squamous cell carcinoma (BrdT overexpression) (M. Scanlan et al., “Expression of cancer-testis antigens in lung cancer: definition of bromodomain testis-specific gene (BRDT) as a new CT gene, CT9,” Cancer Lett 150(2):55-64 (2000)), and colon cancer (Brd4) (R. Rodriguez et al., “Aberrant epigenetic regulation of bromodomain BRD4 in human colon cancer,” J Mol Med (Berl) 90(5):587-95 (2012)).

In one embodiment, because BET inhibitors decrease Brd-dependent recruitment of pTEFb to genes involved in cell proliferation, BET inhibitor compounds of Formula I, Formula Ia, or Formula Ib, or stereoisomers, tautomers, pharmaceutically acceptable salts, or hydrates thereof, or compositions comprising one or more of those compounds may be used to treat cancers that rely on pTEFb (Cdk9/cyclin T) and BET proteins to regulate oncogenes. These cancers include, but are not limited to, chronic lymphocytic leukemia and multiple myeloma (W. Tong et al., “Phase I and pharmacologic study of SNS-032, a potent and selective Cdk2, 7, and 9 inhibitor, in patients with advanced chronic lymphocytic leukemia and multiple myeloma,” J Clin Oncol 28(18):3015-22 (2010)), follicular lymphoma, diffuse large B cell lymphoma with germinal center phenotype, Burkitt's lymphoma, Hodgkin's lymphoma, follicular lymphomas and activated, anaplastic large cell lymphoma (C. Bellan et al., “CDK9/CYCLIN T1 expression during normal lymphoid differentiation and malignant transformation,” J Pathol 203(4):946-52 (2004)), neuroblastoma and primary neuroectodermal tumor (G. De Falco et al., “Cdk9 regulates neural differentiation and its expression correlates with the differentiation grade of neuroblastoma and PNET tumors,” Cancer Biol Ther 4(3):277-81 (2005)), rhabdomyosarcoma (C. Simone and A. Giordano, “Abrogation of signal-dependent activation of the cdk9/cyclin T2a complex in human RD rhabdomyosarcoma cells,” Cell Death Differ 14(1):192-5 (2007)), prostate cancer (D. Lee et al., “Androgen receptor interacts with the positive elongation factor P-TEFb and enhances the efficiency of transcriptional elongation,” J Biol Chem 276(13):9978-84 (2001)), and breast cancer (K. Bartholomeeusen et al., “BET bromodomain inhibition activates transcription via a transient release of P-TEFb from 7SK snRNP,” J Biol Chem (2012)).

In one embodiment, BET inhibitor compounds of Formula I, Formula Ia, or Formula Ib, or stereoisomers, tautomers, pharmaceutically acceptable salts, or hydrates thereof, or compositions comprising one or more of those compounds may be used to treat cancers in which BET-responsive genes, such as CDK6, Bcl2, TYRO3, MYB, and hTERT are up-regulated. M. Dawson et al., Nature 478(7370):529-33 (2011); J. Delmore et al., Cell 146(6):904-17 (2010). These cancers include, but are not limited to, pancreatic cancer, breast cancer, colon cancer, glioblastoma, adenoid cystic carcinoma, T-cell prolymphocytic leukemia, malignant glioma, bladder cancer, medulloblastoma, thyroid cancer, melanoma, multiple myeloma, Barret's adenocarcinoma, hepatoma, prostate cancer, pro-myelocytic leukemia, chronic lymphocytic leukemia, mantle cell lymphoma, diffuse large B-cell lymphoma, small cell lung cancer, and renal carcinoma. M. Ruden and N. Purl, “Novel anticancer therapeutics targeting telomerase,” Cancer Treat Rev (2012); P. Kelly and A. Strasser, “The role of Bcl-2 and its pro-survival relatives in tumourigenesis and cancer therapy” Cell Death Differ 18(9):1414-24 (2011); T. Uchida et al., “Antitumor effect of bcl-2 antisense phosphorothioate oligodeoxynucleotides on human renal-cell carcinoma cells in vitro and in mice,” Mol Urol 5(2):71-8 (2001).

Published and proprietary data have shown direct effects of BET inhibition on cell proliferation in various cancers. In one embodiment, BET inhibitor compounds of Formula I, Formula Ia, Formula Ib, or stereoisomers, tautomers, pharmaceutically acceptable salts, or hydrates thereof, or compositions comprising one or more of those compounds may be used to treat cancers for which exist published and, for some, proprietary, in vivo and/or in vitro data showing a direct effect of BET inhibition on cell proliferation. These cancers include NMC (NUT-midline carcinoma), acute myeloid leukemia (AML), acute B lymphoblastic leukemia (B-ALL), Burkitt's Lymphoma, B-cell Lymphoma, Melanoma, mixed lineage leukemia, multiple myeloma, pro-myelocytic leukemia (PML), and non-Hodgkin's lymphoma. P. Filippakopoulos et al., Nature 468(7327):1067-73 (2010); M. Dawson et al., Nature 478(7370):529-33 (2011); Zuber, J., et al., “RNAi screen identifies Brd4 as a therapeutic target in acute myeloid leukaemia,” Nature 478(7370):524-8 (2011); M. Segura, et al., Cancer Research. 72(8): Supplement 1 (2012). The compounds of the invention have a demonstrated BET inhibition effect on cell proliferation in vitro for the following cancers: Neuroblastoma, Medulloblastoma, lung carcinoma (NSCLC, SCLC), and colon carcinoma.

In one embodiment, because of potential synergy or additive effects between BET inhibitors and other cancer therapy, BET inhibitor compounds of Formula I, Formula Ia, or Formula Ib, or stereoisomers, tautomers, pharmaceutically acceptable salts, or hydrates thereof, or compositions comprising one or more of those compounds may be combined with other therapies, chemotherapeutic agents, or anti-proliferative agents to treat human cancer and other proliferative disorders. The list of therapeutic agents which can be combined with BET inhibitors in cancer treatment includes, but is not limited to, ABT-737, Azacitidine (Vidaza), AZD1152 (Barasertib), AZD2281 (Olaparib), AZD6244 (Selumetinib), BEZ235, Bleomycin Sulfate, Bortezomib (Velcade), Busulfan (Myleran), Camptothecin, Cisplatin, Cyclophosphamide (Clafen), CYT387, Cytarabine (Ara-C), Dacarbazine, DAPT (GSI-IX), Decitabine, Dexamethasone, Doxorubicin (Adriamycin), Etoposide, Everolimus (RAD001), Flavopiridol (Alvocidib), Ganetespib (STA-9090), Gefitinib (Iressa), Idarubicin, Ifosfamide (Mitoxana), IFNa2a (Roferon A), Melphalan (Alkeran), Methazolastone (temozolomide), Metformin, Mitoxantrone (Novantrone), Paclitaxel, Phenformin, PKC412 (Midostaurin), PLX4032 (Vemurafenib), Pomalidomide (CC-4047), Prednisone (Deltasone), Rapamycin, Revlimid (Lenalidomide), Ruxolitinib (INCB018424), Sorafenib (Nexavar), SU11248 (Sunitinib), SU11274, Vinblastine, Vincristine (Oncovin), Vinorelbine (Navelbine), Vorinostat (SAHA), and WP1130 (Degrasyn).

In one embodiment, BET inhibitor compounds of Formula I, Formula Ia, or Formula Ib, or stereoisomers, tautomers, pharmaceutically acceptable salts, or hydrates thereof, or compositions comprising one or more of those compounds may be used to treat benign proliferative and fibrotic disorders, including benign soft tissue tumors, bone tumors, brain and spinal tumors, eyelid and orbital tumors, granuloma, lipoma, meningioma, multiple endocrine neoplasia, nasal polyps, pituitary tumors, prolactinoma, pseudotumor cerebri, seborrheic keratoses, stomach polyps, thyroid nodules, cystic neoplasms of the pancreas, hemangiomas, vocal cord nodules, polyps, and cysts, Castleman disease, chronic pilonidal disease, dermatofibroma, pilar cyst, pyogenic granuloma, juvenile polyposis syndrome, idiopathic pulmonary fibrosis, renal fibrosis, post-operative stricture, keloid formation, scleroderma, and cardiac fibrosis. X. Tanget al., Am J Pathology in press (2013).

In one embodiment, because of their ability to up-regulate ApoA-1 transcription and protein expression (O. Mirguet et al., Bioorg Med Chem Lett 22(8):2963-7 (2012); C. Chung et al., J Med Chem 54(11):3827-38 (2011)), BET inhibitor compounds of Formula I, Formula Ia, or Formula Ib, or stereoisomers, tautomers, pharmaceutically acceptable salts, or hydrates thereof, or compositions comprising one or more of those compounds may be used to treat cardiovascular diseases that are generally associated with including dyslipidemia, atherosclerosis, hypercholesterolemia, and metabolic syndrome (A. Belkina and G. Denis, Nat Rev Cancer 12(7):465-77 (2012); G. Denis Discov Med 10(55):489-99 (2010)). In another embodiment, BET inhibitor compounds of Formula I, Formula Ia, or Formula Ib, or stereoisomers, tautomers, pharmaceutically acceptable salts, or hydrates thereof may be used to treat non-cardiovascular disease characterized by deficits in ApoA-1, including Alzheimer's disease. D. Elliott et al., Clin Lipidol 51(4):555-573 (2010).

In one embodiment, BET inhibitor compounds of Formula I, Formula Ia, or Formula Ib, or stereoisomers, tautomers, pharmaceutically acceptable salts, or hydrates thereof, or compositions comprising one or more of those compounds may be used in patients with insulin resistance and type II diabetes. A. Belkina and G. Denis, Nat Rev Cancer 12(7):465-77 (2012); G. Denis Discov Med 10(55):489-99 (2010); F. Wang et al., Biochem J 425(1):71-83 (2010); G. Denis et al, FEBS Lett 584(15):3260-8 (2010). The anti-inflammatory effects of BET inhibition would have additional value in decreasing inflammation associated with diabetes and metabolic disease. K. Alexandraki et al., “Inflammatory process in type 2 diabetes: The role of cytokines,” Ann N Y Acad Sci 1084:89-117 (2006).

In one embodiment, because of their ability to down-regulate viral promoters, BET inhibitor compounds of Formula I, Formula Ia, or Formula Ib, or stereoisomers, tautomers, pharmaceutically acceptable salts, or hydrates thereof, or compositions comprising one or more of those compounds may be used as therapeutics for cancers that are associated with viruses including Epstein-Barr Virus (EBV), hepatitis virus (HBV, HCV), Kaposi's sarcoma associated virus (KSHV), human papilloma virus (HPV), Merkel cell polyomavirus, and human cytomegalovirus (CMV). D. Gagnon et al., J Virol 83(9):4127-39 (2009); J. You et al., J Viral 80(18):8909-19 (2006); R. Palermo et al., “RNA polymerase II stalling promotes nucleosome occlusion and pTEFb recruitment to drive immortalization by Epstein-Barr virus,” PLoS Pathog 7(10):e1002334 (2011); E. Poreba et al., “Epigenetic mechanisms in virus-induced tumorigenesis,” Clin Epigenetics 2(2):233-47. 2011. In another embodiment, because of their ability to reactivate HIV-1 in models of latent T cell infection and latent monocyte infection, BET inhibitors could be used in combination with anti-retroviral therapeutics for treating HIV. J. Zhu, et al., Cell Rep (2012); C. Banerjee et al., J Leukoc Biol (2012); K. Bartholomeeusen et al., J Biol Chem (2012); Z. Li et al., Nucleic Acids Res (2012.)

In one embodiment, because of the role of epigenetic processes and bromodomain-containing proteins in neurological disorders, BET inhibitor compounds of Formula I, Formula Ia, or Formula Ib, or stereoisomers, tautomers, pharmaceutically acceptable salts, or hydrates thereof, or compositions comprising one or more of those compounds may be used to treat diseases including, but not limited to, Alzheimer's disease, Parkinson's disease, Huntington disease, bipolar disorder, schizophrenia, Rubinstein-Taybi syndrome, and epilepsy. R. Prinjha et al., Trends Pharmacol Sci 33(3):146-53 (2012); S. Muller et al., “Bromodomains as therapeutic targets,” Expert Rev Mol Med 13:e29 (2011).

In one embodiment, because of the effect of BRDT depletion or inhibition on spermatid development, BET inhibitor compounds of Formula I, Formula Ia, or Formula Ib, or stereoisomers, tautomers, pharmaceutically acceptable salts, or hydrates thereof, or compositions comprising one or more of those compounds may be used as reversible, male contraceptive agents. M. Matzuk et al., “Small-Molecule Inhibition of BRDT for Male Contraception,” Cell 150(4): p. 673-684 (2012); B. Berkovits et al., “The testis-specific double bromodomain-containing protein BRDT forms a complex with multiple spliceosome components and is required for mRNA splicing and 3′-UTR truncation in round spermatids,” Nucleic Acids Res 40(15):7162-75 (2012).

### Pharmaceutical Compositions

Another aspect of the invention provides pharmaceutical compositions comprising at least one compound of Formula I, Formula Ia, or Formula Ib as described herein, or tautomer, stereoisomer, pharmaceutically acceptable salt or hydrate thereof formulated together with one or more pharmaceutically acceptable carriers. These formulations include those suitable for oral, rectal, topical, buccal and parenteral (e.g., subcutaneous, intramuscular, intradermal, or intravenous) administration. The most suitable form of administration in any given case will depend on the degree and severity of the condition being treated and on the nature of the particular compound being used.

Formulations suitable for oral administration may be presented in discrete units, such as, e.g., capsules, cachets, lozenges, or tablets, each containing a predetermined amount of a compound of the present disclosure as powder or granules; as a solution or a suspension in an aqueous or non-aqueous liquid; or as an oil-in-water or water-in-oil emulsion. As indicated, such formulations may be prepared by any suitable method of pharmacy which includes the step of bringing into association at least one compound of the present disclosure as the active compound and a carrier or excipient (which may constitute one or more accessory ingredients). The carrier must be acceptable in the sense of being compatible with the other ingredients of the formulation and must not be deleterious to the recipient. The carrier may be a solid or a liquid, or both, and may be formulated with at least one compound described herein as the active compound in a unit-dose formulation, for example, a tablet, which may contain from about 0.05% to about 95% by weight of the at least one active compound. Other pharmacologically active substances may also be present including other compounds. The formulations of the present disclosure may be prepared by any of the well-known techniques of pharmacy consisting essentially of admixing the components.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmacologically administrable compositions can, for example, be prepared by, for example, dissolving or dispersing, at least one active compound of the present disclosure as described herein and optional pharmaceutical adjuvants in an excipient, such as, for example, water, saline, aqueous dextrose, glycerol, ethanol, and the like, to thereby form a solution or suspension. In general, suitable formulations may be prepared by uniformly and intimately admixing the at least one active compound of the present disclosure with a liquid or finely divided solid carrier, or both, and then, if necessary, shaping the product. For example, a tablet may be prepared by compressing or molding a powder or granules of at least one compound of the present disclosure, which may be optionally combined with one or more accessory ingredients. Compressed tablets may be prepared by compressing, in a suitable machine, at least one compound of the present disclosure in a free-flowing form, such as, e.g., a powder or granules, which may be optionally mixed with a binder, lubricant, inert diluent and/or surface active/dispersing agent(s). Molded tablets may be made by molding, in a suitable machine, where the powdered form of at least one compound of the present disclosure is moistened with an inert liquid diluent.

Formulations suitable for buccal (sub-lingual) administration include lozenges comprising at least one compound of the present disclosure in a flavored base, usually sucrose and acacia or tragacanth, and pastilles comprising the at least one compound in an inert base such as, e.g., gelatin and glycerin or sucrose and acacia.

Formulations of the present disclosure suitable for parenteral administration comprise sterile aqueous preparations of at least one compound of Formula I, Formula Ia, or Formula Ib or a tautomer, stereoisomer, pharmaceutically acceptable salt, or hydrate thereof, which are approximately isotonic with the blood of the intended recipient. These preparations are administered intravenously, although administration may also be effected by means of subcutaneous, intramuscular, or intradermal injection. Such preparations may conveniently be prepared by admixing at least one compound described herein with water and rendering the resulting solution sterile and isotonic with the blood. Injectable compositions according to the present disclosure may contain from about 0.1 to about 5% w/w of the active compound.

Formulations suitable for rectal administration are presented as unit-dose suppositories. These may be prepared by admixing at least one compound as described herein with one or more conventional solid carriers, for example, cocoa butter, and then shaping the resulting mixture.

Formulations suitable for topical application to the skin may take the form of an ointment, cream, lotion, paste, gel, spray, aerosol, or oil. Carriers and excipients which may be used include Vaseline, lanoline, polyethylene glycols, alcohols, and combinations of two or more thereof. The active compound (i.e., at least one compound of Formula I, Formula Ia, or Formula Ib or tautomers, stereoisomers, pharmaceutically acceptable salts, and hydrates thereof) is generally present at a concentration of from about 0.1% to about 15% w/w of the composition, for example, from about 0.5 to about 2%.

The amount of active compound administered may be dependent on the subject being treated, the subject's weight, the manner of administration and the judgment of the prescribing physician. For example, a dosing schedule may involve the daily or semi-daily administration of the encapsulated compound at a perceived dosage of about 1 μg to about 1000 mg. In another embodiment, intermittent administration, such as, e.g., on a monthly or yearly basis, of a dose of the encapsulated compound may be employed. Encapsulation facilitates access to the site of action and allows the administration of the active ingredients simultaneously, in theory producing a synergistic effect. In accordance with standard dosing regimens, physicians will readily determine optimum dosages and will be able to readily modify administration to achieve such dosages.

A therapeutically effective amount of a compound or composition disclosed herein can be measured by the therapeutic effectiveness of the compound. The dosages, however, may be varied depending upon the requirements of the patient, the severity of the condition being treated, and the compound being used. In one embodiment, the therapeutically effective amount of a disclosed compound is sufficient to establish a maximal plasma concentration. Preliminary doses as, for example, determined according to animal tests, and the scaling of dosages for human administration is performed according to art-accepted practices.

Toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferable.

Data obtained from the cell culture assays or animal studies can be used in formulating a range of dosage for use in humans. Therapeutically effective dosages achieved in one animal model may be converted for use in another animal, including humans, using conversion factors known in the art (see, e.g., Freireich et al., Cancer Chemother. Reports 50(4):219-244 (1966) and Table 1 for Equivalent Surface Area Dosage Factors).

TABLE 1
Equivalent Surface Area Dosage Factors:

To:

| | Mouse | Rat | Monkey | Dog | Human |
|---|---|---|---|---|---|
| From: | (20 g) | (150 g) | (3.5 kg) | (8 kg) | (60 kg) |
| Mouse | 1 | ½ | ¼ | ⅙ | 1/12 |
| Rat | 2 | 1 | ½ | ¼ | 1/7 |
| Monkey | 4 | 2 | 1 | ⅗ | ⅓ |
| Dog | 6 | 4 | ⅗ | 1 | ½ |
| Human | 12 | 7 | 3 | 2 | 1 |

The dosage of such compounds lies preferably within a range of circulating concentrations that include the ED₅₀ with little or no toxicity. The dosage may vary within this range depending upon the dosage form employed and the route of administration utilized. Generally, a therapeutically effective amount may vary with the subject's age, condition, and gender, as well as the severity of the medical condition in the subject. The dosage may be determined by a physician and adjusted, as necessary, to suit observed effects of the treatment.

In one embodiment, a compound of Formula I, Formula Ia, or Formula Ib or a tautomer, stereoisomer, pharmaceutically acceptable salt or hydrate thereof, is administered in combination with another therapeutic agent. The other therapeutic agent can provide additive or synergistic value relative to the administration of a compound of the present disclosure alone. The therapeutic agent can be, for example, a statin; a PPAR agonist, e.g., a thiazolidinedione or fibrate; a niacin, a RVX, FXR or LXR agonist; a bile-acid reuptake inhibitor; a cholesterol absorption inhibitor; a cholesterol synthesis inhibitor; a cholesteryl ester transfer protein (CETP), an ion-exchange resin; an antioxidant; an inhibitor of AcylCoA cholesterol acytransferase (ACAT inhibitor); a tyrophostine; a sulfonylurea-based drug; a biguanide; an alpha-glucosidase inhibitor; an apolipoprotein E regulator; a HMG-CoA reductase inhibitor, a microsomal triglyceride transfer protein; an LDL-lowing drug; an HDL-raising drug; an HDL enhancer; a regulator of the apolipoprotein A-IV and/or apolipoprotein genes; or any cardiovascular drug.

In another embodiment, a compound of Formula I, Formula Ia, or Formula Ib or a tautomer, stereoisomer, pharmaceutically acceptable salt or hydrate thereof, is administered in combination with one or more anti-inflammatory agents. Anti-inflammatory agents can include immunosuppressants, TNF inhibitors, corticosteroids, non-steroidal anti-inflammatory drugs (NSAIDs), disease-modifying anti-rheumatic drugs (DMARDS), and the like. Exemplary anti-inflammatory agents include, for example, prednisone; methylprenisolone (Medrol®), triamcinolone, methotrexate (Rheumatrex®, Trexall®), hydroxychloroquine (Plaquenil®), sulfasalzine (Azulfidine®), leflunomide (Arava®), etanercept (Enbrel®), infliximab (Remicade®), adalimumab (Humira®), rituximab (Rituxan®), abatacept (Orencia®), interleukin-1, anakinra (Kineret™), ibuprofen, ketoprofen, fenoprofen, naproxen, aspirin, acetominophen, indomethacin, sulindac, meloxicam, piroxicam, tenoxicam, lornoxicam, ketorolac, etodolac, mefenamic acid, meclofenamic acid, flufenamic acid, tolfenamic acid, diclofenac, oxaprozin, apazone, nimesulide, nabumetone, tenidap, etanercept, tolmetin, phenylbutazone, oxyphenbutazone, diflunisal, salsalate, olsalazine, or sulfasalazine.

### LIST OF EXEMPLARY EMBODIMENTS

1. A compound of Formula I:

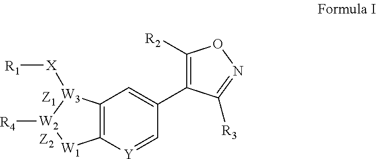

or a stereoisomer, tautomer, pharmaceutically acceptable salt, or hydrate thereof, wherein:
   W₁ is selected from N and NH;
   W₂, and W₃ are selected from C and N;
   Z₁ and Z₂ are selected from a single bond and a double bond;
   R₁ is selected from carbocycle (C₅-C₁₀) and heterocycle (C₂-C₁₀) optionally substituted with 1 to 5 groups independently selected from R₄;
   R₂ is selected from hydrogen and alkyl (C₁-C₆) optionally substituted with halogen and hydroxyl;
   R₃ is selected from alkyl (C₁-C₆) optionally substituted with halogen and hydroxyl, with the proviso that if R₂ and R₃ are methyls, then R₁ is different from:

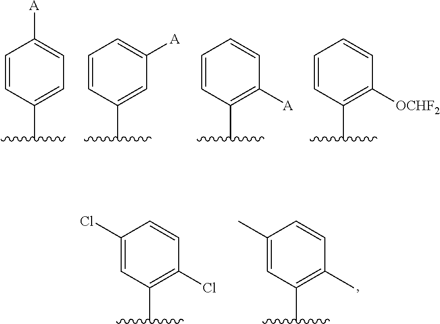

wherein A is selected from hydrogen, halogen, methoxy, —CN, —NO₂, —COOMe, and —CONMe₂;
R₄ if present, is selected from hydrogen, alkyl (C₁-C₁₀), carbocycle (C₃-C₁₀), and heterocycle (C₂-C₁₀) optionally substituted with 1 to 5 groups independently selected from R₅;
each R₅ is independently selected from deuterium, alkyl(C₁-C₆), alkoxy(C₁-C₆), amino, —NHC(O)NH-alkyl(C₁-C₆), halogen, amide, —CF₃, —CN, —N₃, ketone (C₁-C₆), —S(O)— alkyl(C₁-C₄), —SO₂-alkyl(C₁-C₆), thioalkyl(C₁-C₆), —COOH, and ester, each of which may be optionally substituted with hydrogen, F, Cl, Br, —OH, —NH₂, —NHMe, —OMe, —SMe, oxo, and/or thio-oxo;
X is selected from —CH₂— optionally substituted with 1 to 2 groups independently selected from R₅;
Y is selected from N and CH;
wherein if W₃ is C, then W₂ is N, W₁ is NH, Z₁ is a double bond, Z₂ is a single bond, and R₄ is absent; and
wherein if W₃ is N, then W₂ is C, W₁ is N, Z₁ is a single bond and Z₂ is a double bond.
2. The compound according to embodiment 1, wherein the compound is a compound of Formula Ia:

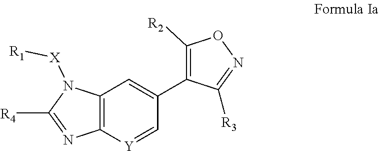

or a stereoisomer, tautomer, pharmaceutically acceptable salt, or hydrate thereof,
wherein:
   R₁ is selected from carbocycle (C₅-C₁₀) and heterocycle (C₂-C₁₀) optionally substituted with 1 to 5 groups independently selected from R₅;
   R₃ is selected from hydrogen and alkyl (C₁-C₆) optionally substituted with halogen and hydroxyl;
   R₃ is selected from alkyl (C₁-C₆) optionally substituted with halogen and hydroxyl, with the proviso that if R₂ and R₃ are methyls, then R₁ is different from:

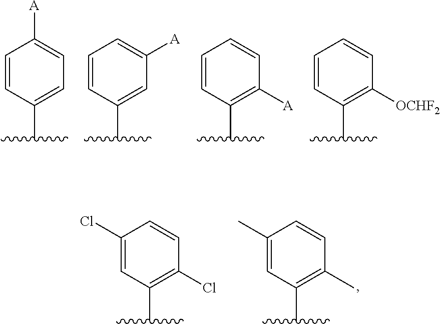

wherein A is selected from hydrogen, halogen, methoxy, —CN, —NO₂, —COOMe, and —CONMe₂;
R₄ is selected from hydrogen, alkyl (C₁-C₁₀), carbocycle (C₃-C₁₀), and heterocycle (C₂-C₁₀) optionally substituted with 1 to 5 groups independently selected from R₅;
each R₅ is independently selected from deuterium, alkyl(C₁-C₆), alkoxy(C₁-C₆), amino, —NHC(O)NH-alkyl(C₁-C₆), halogen, amide, —CF₃, —CN, —N₃, ketone (C₁-C₆), —S(O)— alkyl(C₁-C₄), —SO₂-alkyl(C₁-C₆), thioalkyl(C₁-C₆), —COOH, and ester, each of which may be optionally substituted with hydrogen, F, Cl, Br, —OH, —NH₂, —NHMe, —OMe, —SMe, oxo, and/or thio-oxo;
X is selected from —CH₂— optionally substituted with 1 to 2 groups independently selected from R₅; and
Y is selected from N and CH.
3. The compound according to embodiment 1, wherein the compound is a compound of Formula Ib:

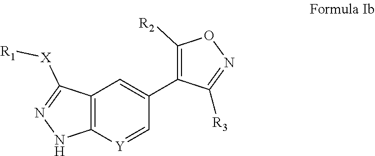

or a stereoisomer, tautomer, pharmaceutically acceptable salt, or hydrate thereof,
wherein:
   R₁ is selected from carbocycle (C₅-C₁₀) and heterocycle (C₂-C₁₀) optionally substituted with 1 to 5 groups independently selected from R₅;
   R₂ is selected from hydrogen and alkyl (C₁-C₆) optionally substituted with halogen and hydroxyl;
   R₃ is selected from alkyl (C₁-C₆) optionally substituted with halogen and hydroxyl, with the proviso that if R₂ and R₃ are methyls, then R₁ is different from:

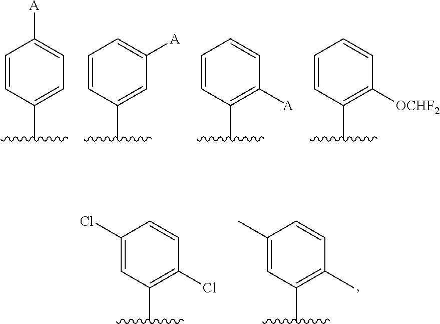

wherein A is selected from hydrogen, halogen, methoxy, —CN, —NO₂, —COOMe, and —CONMe₂;
each R₅ is independently selected from deuterium, alkyl(C₁-C₆), alkoxy(C₁-C₆), amino, —NHC(O)NH-alkyl(C₁-C₆), halogen, amide, —CF₃, —CN, —N₃, ketone (C₁-C₆), —S(O)— alkyl(C₁-C₄), —SO₂-alkyl(C₁-C₆), thioalkyl(C₁-C₆), —COOH, and ester, each of which may be optionally substituted with hydrogen, F, Cl, Br, —OH, —NH₂, —NHMe, —OMe, —SMe, oxo, and/or thio-oxo;
   X is selected from —CH₂— optionally substituted with 1 to 2 groups independently selected from R₅; and
   Y is selected from N and CH.
4. The compound according to any one of embodiments 1 to 3, wherein R₁ is selected from carbocycle (C₅-C₁₀) optionally substituted with 1 to 5 groups independently selected from R₅.
5. The compound according to any one of embodiments 1 to 4, wherein R₁ is selected from phenyl groups optionally substituted with 1 to 5 groups independently selected from R₅.
6. The compound according to any one of embodiments 1 to 5, wherein R₁ is selected from phenyl groups substituted with 1 to 5 groups independently selected from thioalkyl(C₁-C₆), ester, —S(O)-alkyl(C₁-C₄), and —COOH.
7. The compound according to any one of embodiments 1 to 6, wherein R₁ is selected from phenyl groups substituted with 1 to 5 groups independently selected from —SMe, —C(O)OMe, —S(O)Me, and —COOH.
8. The compound according to any one of embodiments 1 to 5, wherein R₁ is unsubstituted phenyl.
9. The compound according to any one of embodiments 1 to 3, wherein R₁ is selected from heterocycles (C₂-C₁₀) optionally substituted with 1 to 5 groups independently selected from R₅.
10. The compound according to any one of embodiments 1 to 3 and 9, wherein R₁ is selected from:

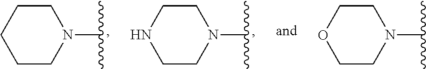

optionally substituted with 1 to 5 groups independently selected from R₅.
11. The compound according to any one of embodiments 1 to 3, 9, and 10, wherein R₁ is selected from:

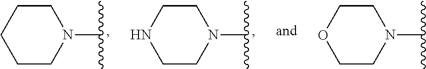

optionally substituted with 1 to 5 groups independently selected from —CN, alkyl(C₁-C₆), alkoxy(C₁-C₆), and halogen.
12. The compound according to any one of embodiments 1 to 3, and 9-11, wherein R₁ is selected from:

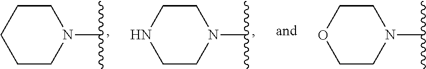

optionally substituted with 1 to 5 groups independently selected from —CN, methyl, methoxy, and F.
13. The compound according to any one of embodiments 1 to 12, wherein R₁ is selected from:

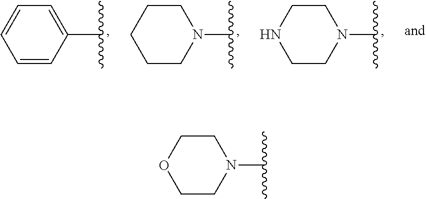

optionally substituted with 1 to 5 groups independently selected from R₅.
14. The compound according to any one of embodiments 1 to 13, wherein R₁ is selected from:

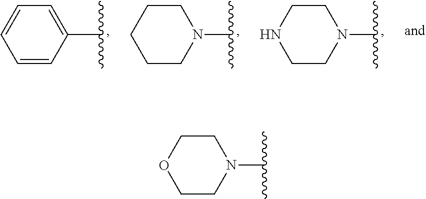

optionally substituted with 1 to 5 groups independently selected from thioalkyl(C₁-C₆), ester, —S(O)-alkyl(C₁-C₄), —COOH, —CN, alkyl(C₁-C₆), alkoxy(C₁-C₆), and halogen.
15. The compound according to any one of embodiments 1 to 14, wherein R₁ is selected from:

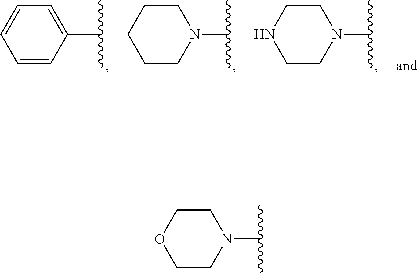

optionally substituted with 1 to 5 groups independently selected from —SMe, —C(O)OMe, —S(O)Me, —COOH, —CN, methyl, methoxy, and F.
16. The compound according to any one of embodiments 1 to 15, wherein R₁ is selected from the following groups:

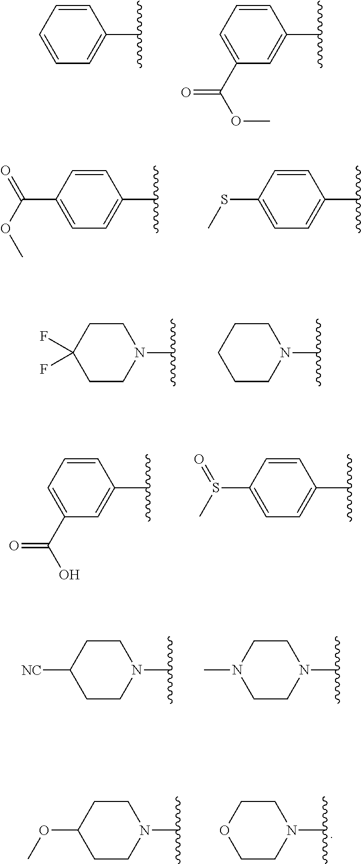

17. The compound according to any one of embodiments 1 to 16, wherein R₂ is selected from alkyl (C₁-C₆) optionally substituted with halogen and/or hydroxyl.
18. The compound according to any one of embodiments 1 to 17, wherein R₂ is is a methyl group optionally substituted with halogen and/or hydroxyl.
19. The compound according to any one of embodiments 1 to 17, wherein R₂ is is a methyl group.
20. The compound according to any one of embodiments 1 to 16, wherein R₂ is hydrogen.
21. The compound according to any one of embodiments 1 to 20, wherein R₃ is selected from alkyl (C₁-C₆) optionally substituted with halogen and/or hydroxyl.
22. The compound according to any one of embodiments 1 to 21, wherein R₃ is a methyl group optionally substituted with halogen and/or hydroxyl.
23. The compound according to any one of embodiments 1 to 22, wherein R₃ is a methyl group.
24. The compound according to any one of embodiments 1 to 19 and 21 to 23, wherein R₂ and R₃ are methyl groups independently and optionally substituted with halogen and/or hydroxyl.
25. The compound according to any one of embodiments 1 to 19 and 21 to 24, wherein R₂ and R₃ are methyl groups.
26. The compound according to any one of embodiments 1 to 16, 20, and 21 to 25, wherein R₂ is hydrogen and R₃ is selected from alkyl (C₁-C₆) optionally substituted with halogen and/or hydroxyl.
27. The compound according to any one of embodiments 1 to 16, 20, and 21 to 26, wherein R₂ is hydrogen and R₃ is selected from methyl optionally substituted with halogen and/or hydroxyl.
28. The compound according to any one of embodiments 1, 2, and 4 to 27, wherein R₄ is hydrogen.
29. The compound according to any one of embodiments 1, 2, and 4 to 27, wherein R₄ is selected from alkyl (C₁-C₆) optionally substituted with 1 to 5 groups independently selected from R₅.
30. The compound according to any one of embodiments 1, 2, 4 to 27, and 29, wherein R₄ is selected from methyl optionally substituted with 1 to 5 groups independently selected from R₅.
31. The compound according to any one of embodiments 1, 2, 4 to 27, 29, and 30, wherein R₄ is methyl.
32. The compound according to any one of embodiments 1, 2, and 4 to 27, wherein R₄ is selected from heterocycle (C₂-C₆) optionally substituted with 1 to 5 groups independently selected from R₅.
33. The compound according to any one of embodiments 1, 2, and 4 to 27, wherein R₄ is selected from carbocycle (C₃-C₁₀) optionally substituted with 1 to 5 groups independently selected from R₅.
34. The compound according to any one of embodiments 1 to 33, wherein each R₅ is independently selected from deuterium, alkyl(C₁—C), alkoxy(C₁-C₆), amino, —NHC(O)NH— alkyl(C₁-C₆), halogen, amide, —CF₃, —CN, —N₃, ketone (C₁-C₆), —S(O)-alkyl(C₁-C₄), —SO₂-alkyl(C₁-C₆), and thioalkyl(C₁-C₆).
35. The compound according to any one of embodiments 1 to 34, wherein each R₅ is independently selected from thioalkyl(C₁-C₆), ester, —S(O)-alkyl(C₁-C₄), —COOH, —CN, alkyl(C₁-C₆), alkoxy(C₁-C₆), and halogen.
36. The compound according to any one of embodiments 1 to 35, wherein Y is CH.
37. The compound according to any one of embodiments 1 to 35, wherein Y is N.
38. The compound according to any one of embodiments 1 to 37, wherein X is CH₂.
39. The compound according to embodiment 1 or embodiment 2, wherein the compound is selected from:
3,5-Dimethyl-4-(2-methyl-1-(4-(methylthio)benzyl)-1H-imidazo[4,5-b]pyridin-6-yl)isoxazole;
Methyl 3-((6-(3,5-dimethylisoxazol-4-yl)-2-methyl-1H-imidazo[4,5-b]pyridin-1-yl)methyl)benzoate;
Methyl 4-((6-(3,5-dimethylisoxazol-4-yl)-2-methyl-1H-imidazo[4,5-b]pyridin-1-yl)methyl)benzoate;
3,5-Dimethyl-4-(2-methyl-1-(3-(methylthio)benzyl)-1H-imidazo[4,5-b]pyridin-6-yl)isoxazole;
3,5-Dimethyl-4-(2-methyl-1-(4-(methylsulfinyl)benzyl)-1H-imidazo[4,5-b]pyridin-6-yl)isoxazole;
3-((6-(3,5-Dimethylisoxazol-4-yl)-2-methyl-1H-imidazo[4,5-b]pyridin-1-yl)methyl)benzoic acid;
(4-(1-Benzyl-2-methyl-1H-imidazo[4,5-b]pyridin-6-yl)-3-methylisoxazol-5-yl)methanol;
4-(1-Benzyl-2-methyl-1H-imidazo[4,5-b]pyridin-6-yl)-3-methylisoxazole;
   and stereoisomers, tautomers, pharmaceutically acceptable salts, or hydrates thereof.
40. The compound according to embodiment 1 or embodiment 3, wherein the compound is selected from:
4-(3-((4,4-Difluoropiperidin-1-yl)methy)-1H-indazol-5-yl)-3, methylisoxazole;
3,5-Dimethyl-4-[3-(1-piperidylmethyl)-1H-indazol-5-yl]isoxazole;
3,5-Dimethyl-4-[3-[(4-methylpiperazin-1-yl)methyl]-1H-indazol-5-yl]isoxazole;
1-[[5-(3,5-Dimethylisoxazol-4-yl)-1H-indazol-3-yl]methyl]piperidine-4-carbonitrile;
4-[3-[(4-Methoxy-1-piperidyl)methyl]-1H-indazol-5-yl]-3,5-dimethyl-isoxazole;
4-[[5-(3,5-Dimethylisoxazol-4-yl)-1H-indazol-3-yl]methyl]morpholine;
   and stereoisomers, tautomers, pharmaceutically acceptable salts, or hydrates thereof.
41. A pharmaceutical composition comprising the compound of any one of embodiments 1-40, and a pharmaceutically acceptable carrier.
42. A method for inhibition of BET protein function comprising administering a therapeutically effective amount of the compound of any one of embodiments 1-40 or a pharmaceutical composition according to embodiment 41.
43. A method of treating an autoimmune or inflammatory disorder associated with BET proteins comprising administering a therapeutically effective amount of the compound of any one of embodiments 1-40 or a pharmaceutical composition according to embodiment 41.
44. The method of embodiment 43, wherein the autoimmune or inflammatory disorder is selected from Acute Disseminated Encephalomyelitis, Agammaglobulinemia, Allergic Disease, Ankylosing spondylitis, Anti-GBM/Anti-TBM nephritis, Anti-phospholipid syndrome, Autoimmune aplastic anemia, Autoimmune hepatitis, Autoimmune inner ear disease, Autoimmune myocarditis, Autoimmune pancreatitis, Autoimmune retinopathy, Autoimmune thrombocytopenic purpura, Behcet's Disease, Bullous pemphigoid, Castleman's Disease, Celiac Disease, Churg-Strauss syndrome, Crohn's Disease, Cogan's syndrome, Dry eye syndrome, Essential mixed cryoglobulinemia, Dermatomyositis, Devic's Disease, Encephalitis, Eosinophlic esophagitis, Eosinophilic fasciitis, Erythema nodosum, Giant cell arteritis, Glomerulonephritis, Goodpasture's syndrome, Granulomatosis with Polyangiitis (Wegener's), Graves' Disease, Guillain-Barre syndrome, Hashimoto's thyroiditis, Hemolytic anemia, Henoch-Schonlein purpura, idiopathic pulmonary fibrosis, IgA nephropathy, Inclusion body myositis, Type I diabetes, Interstitial cystitis, Kawasaki's Disease, Leukocytoclastic vasculitis, Uchen planus, Lupus (SLE), Microscopic polyangitis, Multiple sclerosis, Myasthenia gravis, myositis, Optic neuritis, Pemphigus, POEMS syndrome, Polyarteritis nodosa, Primary biliary cirrhosis, Psoriasis, Psoriatic arthritis, Pyoderma gangrenosum, Relapsing polychondritis, Rheumatoid arthritis, Sarcoidosis, Scleroderma, Sjogren's syndrome, Takayasu's arteritis, Transverse myelitis, Ulcerative colitis, Uveitis, and Vitiligo.
45. A method of treating an acute or chronic non-autoimmune inflammatory disorder characterized by disregulation of IL-6 and/or IL-17 comprising administering a therapeutically effective amount of the compound of any one of embodiments 1-40 or a pharmaceutical composition according to embodiment 41.
46. The method of embodiment 45, wherein the acute or chronic non-autoimmune inflammatory disorder is selected from sinusitis, pneumonitis, osteomyelitis, gastritis, enteritis, gingivitis, appendicitis, irritable bowel syndrome, tissue graft rejection, chronic obstructive pulmonary disease (COPD), septic shock, osteoarthritis, acute gout, acute lung injury, acute renal failure, burns, Herxheimer reaction, and SIRS associated with viral infections.
47. The method of embodiment 45, wherein the acute or chronic non-autoimmune inflammatory disorder is selected from rheumatoid arthritis (RA) and multiple sclerosis (MS).
48. A method of treating a cancer associated with overexpression, translocation, amplification, or rearrangement of a myc family oncoprotein that is sensitive to BET inhibition comprising administering a therapeutically effective amount of the compound of any one of embodiments 1-40 or a pharmaceutical composition according to embodiment 41.
49. A method of treating a cancer associated with overexpression, translocation, amplification, or rearrangement of BET proteins comprising administering a therapeutically effective amount of the compound of any one of embodiments 1-40 or a pharmaceutical composition according to embodiment 41.
50. A method of treating a cancer that relies on pTEFb (Cdk9/cyclin T) and BET proteins to regulate oncogenes comprising administering a therapeutically effective amount of the compound of any one of embodiments 1-40 or a pharmaceutical composition according to embodiment 41.
51. A method of treating a cancer associated with upregulation of BET responsive genes CDK6, Bcl2, TYRO3, MYB, and hTERT comprising administering a therapeutically effective amount of the compound of any one of embodiments 1-40 or a pharmaceutical composition according to embodiment 41.
52. A method of treating a cancer associated with a gene regulated by a super enhancer comprising administering a therapeutically effective amount of the compound of any one of embodiments 1-40 or a pharmaceutical composition according to embodiment 41.
53. A method of treating a cancer that is sensitive to effects of BET inhibition comprising administering a therapeutically effective amount of the compound of any one of embodiments 1-40 or a pharmaceutical composition according to embodiment 41.
54. A method of treating a cancer that is resistant to treatment with immunotherapy, hormone-deprivation therapy, and/or chemotherapy comprising administering a therapeutically effective amount of the compound of any one of embodiments 1-40 or a pharmaceutical composition according to embodiment 41.
55. The method of any one of embodiments 42-54, wherein the compound of any one of embodiments 1-40 or a pharmaceutical composition according to embodiment 41 is combined with other therapies, chemotherapeutic agents or antiproliferative agents.
56. The method of embodiment 55, wherein the therapeutic agent is selected from ABT-737, Azacitidine (Vidaza), AZD1152 (Barasertib), AZD2281 (Olaparib), AZD6244 (Selumetinib), BEZ235, Bleomycin Sulfate, Bortezomib (Velcade), Busulfan (Myleran), Camptothecin, Cisplatin, Cyclophosphamide (Clafen), CYT387, Cytarabine (Ara-C), Dacarbazine, DAPT (GSI-IX), Decitabine, Dexamethasone, Doxorubicin (Adriamycin), Etoposide, Everolimus (RAD001), Flavopiridol (Alvocidib), Ganetespib (STA-9090), Gefitinib (Iressa), Idarubicin, Ifosfamide (Mitoxana), IFNa2a (Roferon A), Melphalan (Alkeran), Methazolastone (temozolomide), Metformin, Mitoxantrone (Novantrone), Paclitaxel, Phenformin, PKC412 (Midostaurin), PLX4032 (Vemurafenib), Pomalidomide (CC-4047), Prednisone (Deltasone), Rapamycin, Revlimid (Lenalidomide), Ruxolitinib (INCB018424), Sorafenib (Nexavar), SU11248 (Sunitinib), SU11274, Vinblastine, Vincristine (Oncovin), Vinorelbine (Navelbine), Vorinostat (SAHA), and WP1130 (Degrasyn).
57. A method of treating a benign proliferative or fibrotic disorder, selected from the group consisting of benign soft tissue tumors, bone tumors, brain and spinal tumors, eyelid and orbital tumors, granuloma, lipoma, meningioma, multiple endocrine neoplasia, nasal polyps, pituitary tumors, prolactinoma, pseudotumor cerebri, seborrheic keratoses, stomach polyps, thyroid nodules, cystic neoplasms of the pancreas, hemangiomas, vocal cord nodules, polyps, and cysts, Castleman disease, chronic pilonidal disease, dermatofibroma, pilar cyst, pyogenic granuloma, juvenile polyposis syndrome, idiopathic pulmonary fibrosis, renal fibrosis, post-operative stricture, keloid formation, scleroderma, and cardiac fibrosis comprising administering a therapeutically effective amount of the compound of any one of embodiments 1-40 or a pharmaceutical composition according to embodiment 41.
58. A method of treating a disease or disorder that benefits from up-regulation or ApoA-I transcription and protein expression comprising administering a therapeutically effective amount of the compound of any one of embodiments 1-40 or a pharmaceutical composition according to embodiment 41.
59. The method of embodiment 58, wherein the disease is cardiovascular disease, dyslipidemia, atheroschlerosis, hypercholesterolemia, metabolic syndeome, and Alzheimer's disease.
60. A method of treating a cancer associated with a virus comprising administering a therapeutically effective amount of the compound of any one of embodiments 1-40 or a pharmaceutical composition according to embodiment 41.
61. A method for treating HIV infection comprising administering a therapeutically effective amount of the compound of any one of embodiments 1-40 or a pharmaceutical composition according to embodiment 41 alone or in combination with anti-retroviral therapeutic.
62. A method for treating a disease or disorder selected from Alzheimer's disease, Parkinson's disease, Huntington disease, bipolar disorder, schizophrenia, Rubinstein-Taybi syndrome, and epilepsy comprising administering a therapeutically effective amount of the compound of any one of embodiments 1-40 or a pharmaceutical composition according to embodiment 41.
63. A method of male contraception comprising administering a therapeutically effective amount of the compound of any one of embodiments 1-40 or a pharmaceutical composition according to embodiment 41.

### EXAMPLES

General Methods.

Unless otherwise noted, reagents and solvents were used as received from commercial suppliers. Proton nuclear magnetic resonance spectra were obtained on a Bruker 400 MHz spectrometer. Spectra are given in ppm (δ) and coupling constants, J values, are reported in hertz (Hz). Mass spectra analyses were performed on Shimadzu 2020 Mass Spectrometer in ESI or APCI mode when appropriate.

### Abbreviations

AcOH: acetic acid; DCM: dichloromethane; DMF: dimethylformamide; EtOAc: ethyl acetate; EtOH: ethanol; MeOH: methanol; NBS: N-bromosuccinimide; PE: petroleum ether; THF: tetrahydrofuran; TLC: thin layer chromatography.

### General Procedure A:

### Example 1: Preparation of 3,5-Dimethyl-4-(2-methyl-1-(4-(methylthio)benzyl)-1H-imidazo[4,5-b]pyridin-6-yl)isoxazole

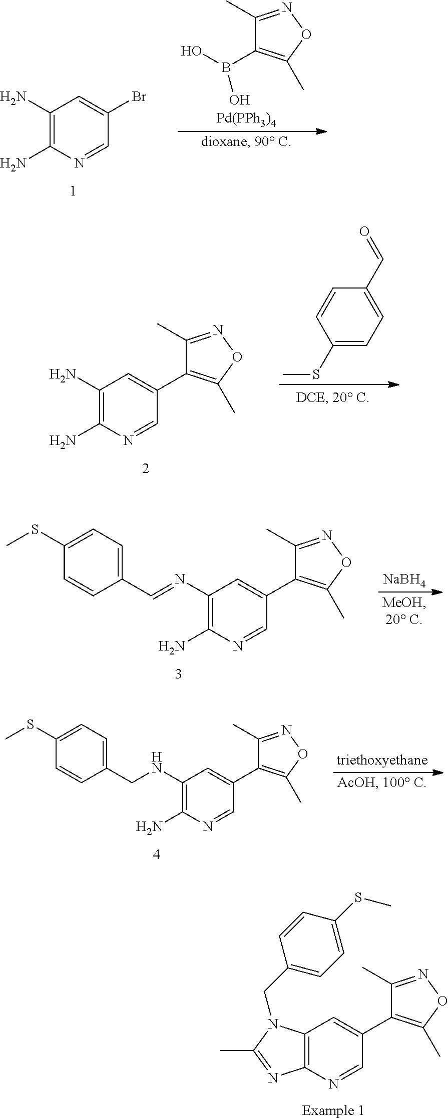

5-Bromopyridine-2,3-diamine (compound 1) (4.0 g, 21.3 mmol, 1.0 eq), (3,5-dimethylisoxazol-4-yl)boronic acid (4.5 g, 31.9 mmol, 1.5 eq), K₂C O₃ (5.9 g, 42.6 mmol, 2.0 eq) and Pd(PPh₃)₄ (1.2 g, 1.1 mmol, 0.05 eq) were combined in a solution of dioxane (60 mL) and H₂O (20 mL). The reaction mixture was degassed with nitrogen and then heated at 90° C. for 16 h. TLC showed the formation of a new more polar product with 15% of 1 remaining. The reaction mixture was partitioned between water (50 mL) and EtOAc (50 mL). The organic phase was separated, washed with water (50 mL), dried over Na₂SO₄, filtered and concentrated under reduced pressure. The residue was purified by column chromatography (50-100% EtOAc/PE) to afford compound 2 (3.2 g, 15.7 mmol, 74% yield) as a yellow solid: ¹H NMR (400 MHz, CDCl₃) δ 2.26 (s, 3H), 2.40 (s, 3H), 3.42 (br. s., 2H), 4.34 (br. s., 2H), 6.80 (d, J=1.88 Hz, 1H), 7.58 (d, J=1.88 Hz, 1H).

To a solution of compound 2 (200 mg, 979 umol, 1.0 eq) and 4-(methylthio)benzaldehyde (149 mg, 979 umol, 1.0 eq) in 1, 2-dichloroethane (20 mL) was added AcOH (200 uL). The mixture was stirred at 20° C. for 16 h. TLC showed the formation of a new less polar product with 30% of 2 remaining. The reaction mixture was partitioned between DCM (15 mL) and sat. aq. NaHCO₃ (15 mL). The organic phase was separated, washed with water (15 mL), dried over Na₂SO₄, filtered and concentrated under reduced pressure. The residue was purified by column chromatography (10-50% EtOAc in PE) to afford compound 3 (150 mg, 443 umol, 45% yield) as a yellow solid.

To a solution of compound 3 (150 mg, 443 umol, 1.0 eq) in MeOH (20 mL) was added NaBH₄ (50 mg, 1.3 mmol, 3.0 eq) in portions at 20° C. The mixture was stirred at 20° C. for 2 h. TLC indicated the consumption of 3 and the clean formation of a new product. The reaction mixture was quenched by addition of water (5 mL) at 0° C. The reaction mixture was diluted with more water (20 mL) and extracted with EtOAc (2×20 mL). The combined organic fractions were washed with water (20 mL), dried over Na₂SO₄, filtered and concentrated under reduced pressure. The residue was purified by column chromatography (50-100% EtOAc in PE) to afford compound 4 (140 mg, 411 umol, 92% yield) as a yellow solid: ¹H NMR (400 MHz, CDCl₃) δ 2.11-2.16 (m, 3H), 2.29 (s, 3H), 2.50 (s, 3H), 3.78 (br. s., 1H), 4.32 (s, 2H), 4.40 (br. s., 2H), 6.59 (d, J=1.88 Hz, 1H), 7.22-7.35 (m, 4H), 7.50 (d, J=1.88 Hz, 1H).

A solution of compound 4 (100 mg, 294 umol, 1.0 eq) in AcOH (3 mL) was added 1,1,1-triethoxyethane (238 mg, 1.5 mmol, 5.0 eq). The mixture was stirred at 100° C. for 1 h. TLC showed the consumption of 4 and the formation of a new spot. The reaction mixture was concentrated under reduced pressure and the residue was partitioned between EtOAc (20 mL) and sat. aq. NaHCO₃ (20 mL). The organic phase was separated, washed with water (20 mL), dried over Na₂SO₄, filtered and concentrated under reduced pressure. The residue was purified by column chromatography (50-100% EtOAc in PE) to afford 3,5-dimethyl-4-(2-methyl-1-(4-(methylthio)benzyl)-1H-imidazo[4,5-b]pyridin-6-yl)isoxazole (Example 1) (55 mg, 149 umol, 51% yield, 98% purity) as a white solid: ¹H NMR (400 MHz, CDCl3) δ 2.21 (s, 3H), 2.37 (s, 3H), 2.48 (s, 3H), 2.75 (s, 3H), 5.34 (s, 2H), 7.03 (d, J=8.41 Hz, 2H), 7.23 (d, J=8.41 Hz, 2H), 7.30 (d, J=2.01 Hz, 1H), 8.41 (d, J=1.76 Hz, 1H); ESI m/z 365.1[M+1]⁺.

### Example 2: Preparation of Methyl 3-((6-(3,5-dimethylisoxazol-4-yl)-2-methyl-1H-imidazo[4,5-b]pyridin-1-yl)methyl)benzoate

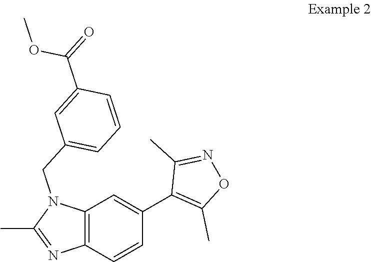 [SMILES: COC(=O)c1cccc(Cn2c(C)nc3ccc(-c4c(C)noc4C)cc32)c1]

Example 2 was synthesized according to General Procedure A substituting methyl 3-formylbenzoate in place of 4-(methylthio)benzaldehyde. 55 mg of white solid isolated: ¹H NMR (400 MHz, CDCl3) δ 2.19 (s, 3H), 2.36 (s, 3H), 2.75 (s, 3H), 3.92 (s, 3H), 5.43 (s, 2H), 7.25 (d, J=7.53 Hz, 1H), 7.31 (d, J=1.76 Hz, 1H), 7.46 (t, J=7.78 Hz, 1H), 7.90 (s, 1H), 8.03 (d, J=7.78 Hz, 1H), 8.42 (d, J=1.76 Hz, 1H); ESI m/z 377.2[M+1]⁺.

### Example 3: Preparation of Methyl 4-((6-(3,5-dimethylisoxazol-4-yl)-2-methyl-1H-imidazo[4,5-b]pyridin-1-yl)methyl)benzoate

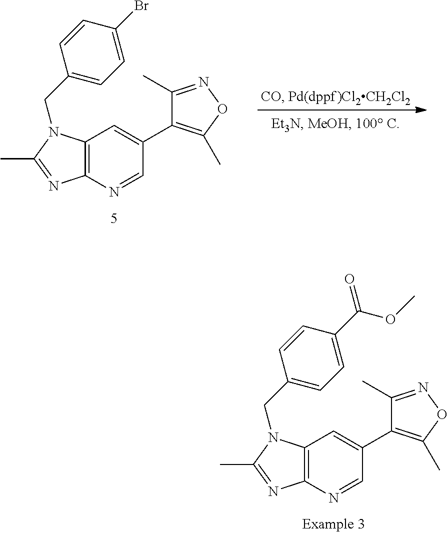 [SMILES: Cc1noc(C)c1-c1cnc2nc(C)n(Cc3ccc(Br)cc3)c2c1 ; COC(=O)c1ccc(Cn2c(C)nc3ncc(-c4c(C)noc4C)cc32)cc1]

Compound 5 was synthesized according to General Procedure A substituting 4-bromobenzaldehyde in place of 4-(methylthio)benzaldehyde. 1.6 g of yellow solid isolated: ¹H NMR (400 MHz, CDCl₃) δ 2.18-2.24 (m, 3H), 2.33-2.39 (m, 3H), 2.67-2.74 (m, 3H), 5.34 (s, 2H), 6.97 (d, J=8.53 Hz, 2H), 7.25-7.32 (m, 2H), 7.47-7.56 (m, 2H), 8.39-8.45 (m, 1H).

To a solution of compound 5 (150 mg, 378 umol, 1.0 eq) and triethylamine (76 mg, 755 umol, 2.0 eq) in methanol (10 mL) was added Pd(dppf)Cl₂.CH₂Cl₂ (31 mg, 38 umol, 0.1 eq) under a nitrogen atmosphere. The suspension was degassed and purged with CO gas 3 times. The mixture was stirred under CO (1 MPa) at 100° C. for 16 h. LC-MS showed about 40% conversion to the expected product. The reaction mixture was cooled, filtered and the filtrate was concentrated under reduced pressure. The residue was purified by preparative HPLC to afford methyl 4-((6-(3,5-dimethylisoxazol-4-yl)-2-methyl-1H-imidazo[4,5-b]pyridin-1-yl)methyl)benzoate hydrochloride (Example 3) (57 mg, 150 umol, 40% yield) as a yellow solid: ¹H NMR (400 MHz, Methanol-d₄) δ 2.22 (s, 3H), 2.40-2.44 (m, 3H), 2.94 (s, 3H), 3.92 (s, 3H), 5.87 (s, 2H), 7.47 (d, J=8.41 Hz, 2H), 8.07 (d, J=8.28 Hz, 2H), 8.23 (d, J=1.63 Hz, 1H), 8.65 (s, 1H); ESI m/z 377.1 [M+1]⁺.

### Example 4: Preparation of 3,5-Dimethyl-4-(2-methyl-1-(3-(methylthio)benzyl)-1H-imidazo[4,5-b]pyridin-6-yl)isoxazole

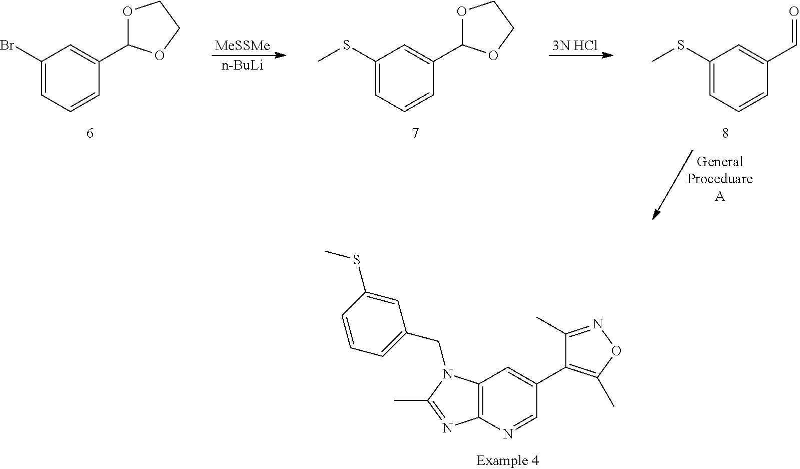

n-BuLi (671 mg, 10.5 mmol, 1.2 eq) was added dropwise to a solution of compound 6 (2.0 g, 8.7 mmol, 1.0 eq) in THF (20 mL) at −78° C. The mixture was stirred at this temperature for 25 min and then 1,2-dimethyldisulfane (1.2 g, 13.1 mmol, 1.5 eq) was added dropwise. The resulting mixture was stirred at −78° C. for 30 min. The reaction mixture was quenched by the addition of saturated aqueous NaHCO₃ (5 mL) at 0° C. The mixture was diluted with more NaHCO₃ (30 mL) and extracted with EtOAc (2×20 mL). The combined organic fractions were washed with brine (20 mL), dried over Na₂SO₄, filtered and concentrated under reduced pressure to afford compound 7 (1.60 g, crude) as yellow oil. The oil was used into the next step without further purification.

A solution of compound 7 (1.6 g, 8.2 mmol, 1.0 eq) in HCl (20 mL, 3M) and CH₃CN (20 mL) was stirred at 20° C. for 2 h. The reaction mixture was adjusted to pH 9 with saturated aqueous NaHCO₃ and extracted with EtOAc (2×20 mL). The combined organic fractions were washed with brine (20 mL), dried over Na₂SO₄, filtered and concentrated under reduced pressure to afford 8, 3-(methylthio)benzaldehyde, (1.2 g, crude) as yellow oil. The oil was used into the next step without further purification: ¹H NMR (400 MHz, CDCl₃) δ 2.54 (s, 1H), 7.41-7.52 (m, 2H), 7.62 (dt, J=7.28, 1.38 Hz, 1H), 7.73 (t, J=1.44 Hz, 1H), 9.98 (s, 1H).

Example 4 was synthesized according to General Procedure A substituting 3-(methylthio)benzaldehyde in place of 4-(methylthio)benzaldehyde. 60 mg of yellow solid was isolated as a hydrochloride salt: ¹H NMR (400 MHz, Methanol-d₄) δ 2.23 (s, 3H), 2.42 (s, 3H), 2.49 (s, 3H), 3.01 (s, 3H), 5.77 (s, 2H), 7.11 (d, J=7.53 Hz, 1H), 7.28-7.33 (m, 1H), 7.33-7.40 (m, 2H), 8.21 (d, J=1.88 Hz, 1H), 8.67 (d, J=1.76 Hz, 1H); ESI m/z 365.2[M+1]⁺.

### Example 5: Preparation of 3,5-Dimethyl-4-(2-methyl-1-(4-(methylsulfinyl)benzyl)-1H-imidazo[4,5-b]pyridin-6-yl)isoxazole

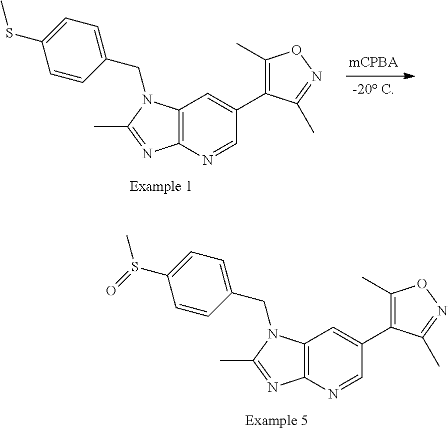 [SMILES: CSc1ccc(Cn2c(C)nc3ncc(-c4c(C)noc4C)cc32)cc1 ; Cc1noc(C)c1-c1cnc2nc(C)n(Cc3ccc(S(C)=O)cc3)c2c1]

A 4 mL vial was charged with Example 1 (35 mg, 96 umol, 1.0 eq) as a solution in DCM (0.75 mL). The vial was cooled to −20′C and a solution of 3-chloroperoxybenzoic acid (21 mg, 106 umol, 1.10 eq) in DCM (0.25 mL) was added. The reaction was stirred at −20° C. for 10 min and was purified by preparative TLC (10% MeOH in EtOAc) to afford Example 5 (18 mg, 46 umol, 48% yield) as a white solid: ¹H NMR (400 MHz, CDCl₃) δ 2.22 (s, 3H) 2.38 (s, 3H) 2.72 (s, 3H) 2.73 (s, 3H) 5.46 (s, 2H) 7.26 (d, J=8.28 Hz, 2H) 7.33 (d, J=2.01 Hz, 1H) 7.67 (d, J=8.41 Hz, 2H) 8.45 (d, J=1.88 Hz, 1H); ESI m/z 381.0 [M+1]⁺.

### Example 6: Preparation of 3-((6-(3,5-Dimethylisoxazol-4-yl)-2-methyl-1H-imidazo[4,5-b]pyridin-1-yl)methyl)benzoic acid

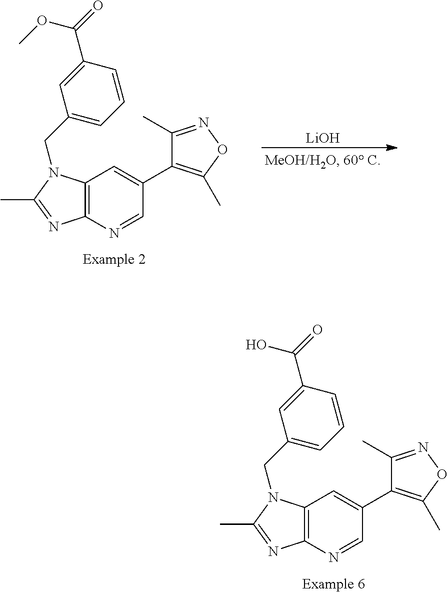 [SMILES: COC(=O)c1cccc(Cn2c(C)nc3ncc(-c4c(C)noc4C)cc32)c1 ; "LiOH" ; Cc1noc(C)c1-c1cnc2nc(C)n(Cc3cccc(C(=O)O)c3)c2c1]

Lithium hydroxide (6 mg, 239 umol, 3.0 eq) was added to a solution of Example 2 (30 mg, 80 umol, 1.0 eq) in methanol (2 mL) and water (500 uL). The mixture was stirred at 60° C. for 10 min and then concentrated under reduced pressure. The residue was purified by preparative HPLC to afford 3-((6-(3,5-dimethylisoxazol-4-yl)-2-methyl-1H-imidazo[4,5-b]pyridin-1-yl)methyl)benzoic acid hydrochloride (Example 6) (20 mg, 55 umol, 68% yield) as a white solid: ¹H NMR (400 MHz, Methanol-d₄) δ 2.24 (s, 3H), 2.42 (s, 3H), 2.99 (s, 3H), 5.87 (s, 2H), 7.52-7.61 (m, 1H), 7.62-7.69 (m, 1H), 8.03-8.10 (m, 2H), 8.32 (d, J=1.76 Hz, 1H), 8.68 (d, J=1.76 Hz, 1H); ESI m/z 363.0 [M+1]⁺.

### Example 7: Preparation of (4-(1-Benzyl-2-methyl-1H-imidazo[4,5-b]pyridin-6-yl)-3-methylisoxazol-5-yl)methanol

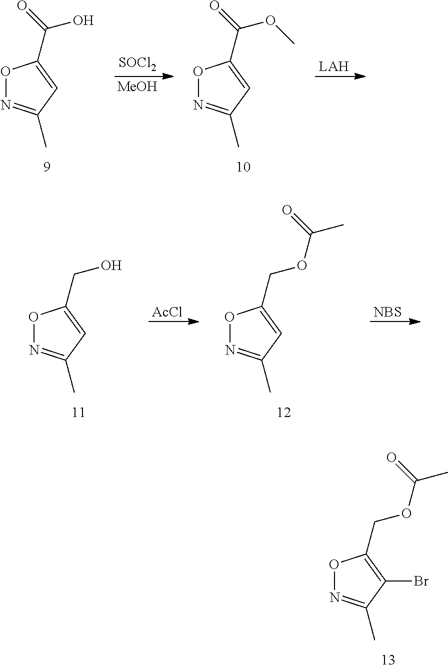

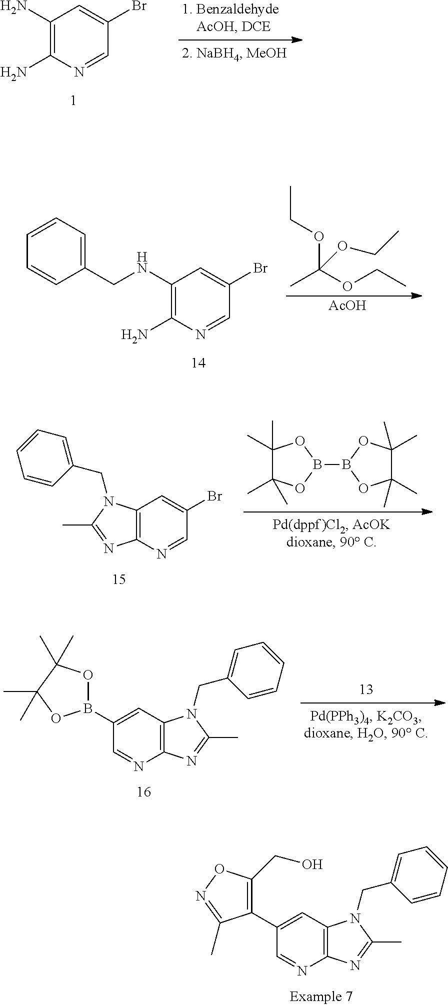

To a solution of compound 9 (1 g, 7.9 mmol, 1.0 eq) in dry MeOH (20 mL) was added SOCl₂ (1 g, 8.7 mmol, 1.1 eq) slowly at 0° C. The reaction mixture was stirred at 25° C. for 15 min and heated to 40° C. for 3 h. The reaction mixture was concentrated under reduced pressure, dissolved in EtOAc (50 mL), washed with brine (2×50 mL), dried over anhydrous Na₂SO₄, filtered and concentrated in vacuum. The residue was purified by column chromatography (0-20% EtOAc in PE) to afford 10 (850 mg, 6.0 mmol, 76% yield) as a white solid: ¹H NMR (400 MHz, CDCl₃) δ 2.40 (s, 3H), 3.98 (s, 3H), 6.82 (s, 1H).

LiAlH₄ (228.5 mg, 6 mmol, 1 eq) was added to a solution of compound 10 (850 mg, 6 mmol, 1 eq) in dry THF (20 mL) at 0° C. under a nitrogen atmosphere. The mixture was stirred at 0° C. for 2 h at which time TLC showed complete consumption of starting material. Water (0.3 mL) was added at 0° C. and the reaction mixture was stirred for 10 min. Then 10% aq. NaOH (0.3 mL) was added and the mixture was stirred for another 10 min. Then more water (0.9 mL) was added. After stirring for 15 min, the reaction mixture was filtered and concentrated under reduced pressure to afford compound 11 (450 mg, 3.9 mmol, 66% yield) as a yellow oil: ¹H NMR (400 MHz, CDCl₃) δ 2.30 (s, 3H), 4.68-4.75 (m, 2H), 6.09 (s, 1H).

To a solution of compound 11 (450 mg, 4 mmol, 1.0 eq) in DCM (20 mL) was added triethylamine (604.1 mg, 6 mmol, 1.5 eq). The reaction was cooled to 0° C. and acetyl chloride (344 mg, 4.4 mmol, 1.1 eq) was added. The reaction mixture was stirred for 3 h at 15° C. Ice water (100 mL) was added and the mixture was stirred for another 10 min. The aqueous phase was extracted with DCM (2×50 mL) and the combined organic fractions were washed with brine (2×100 mL), dried over anhydrous Na₂SO₄, filtered and concentrated under vacuum. The residue was purified by silica gel chromatography (0-20% EtOAc in PE) to afford compound 12 (150 mg, 966.8 μmol, 24% yield) as yellow oil: ¹H NMR (400 MHz, CDCl₃) δ 2.12 (s, 3H), 2.31 (s, 3H), 5.14 (s, 2H), 6.15 (s, 1H).

To a solution of compound 12 (130 mg, 837.9 umol, 1 eq) in acetic acid (1.5 mL) was added NBS (178.9 mg, 1 mmol, 1.2 eq) and sulfuric acid (164 mg, 1.7 mmol, 2 eq). The reaction mixture was heated at 110° C. for 1 h when TLC showed the starting material was consumed completely. The reaction mixture was cooled to 25° C. and carefully poured into a vigorously stirred solution of ice cooled saturated NaHCO₃. The solution (pH 8-9) was extracted with EtOAc (3×20 mL). The combined organic fractions were washed with 2% sodium thiosulfate (50 mL), brine (2×50 mL), dried over anhydrous Na₂SO₄, filtered and concentrated under vacuum. The residue was purified by column chromatography (0-10% EtOAc in PE) to afford compound 13 (60 mg, 256.4 umol, 31% yield) as a yellow oil: ¹H NMR (400 MHz, CDCl₃) δ 2.14 (s, 3H), 2.32 (s, 3H), 5.17 (s, 2H).

A solution of compound 1 (1 g, 5.3 mmol, 1 eq), benzaldehyde (621 mg, 5.8 mmol, 1.1 eq) and acetic acid (700 uL) in DCE (60 mL) was stirred at 15° C. for 15 h. The reaction was diluted with DCM, washed with sat. aq. NaHCO₃ (3×100 mL), dried over anhydrous Na₂SO₄, filtered and concentrated to afford an intermediate imine. The imine was dissolved in MeOH (20 mL), and sodium borohydride (261.6 mg, 6.9 mmol, 1.3 eq) was added. The reaction mixture was stirred at 15° C. for 2 h when TLC showed the imine was consumed. The reaction mixture was concentrated and the residue was purified by column chromatography (20-50% EtOAc in PE) to afford 14 (750 mg, 2.7 mmol, 51% yield) as an orange-yellow solid: ¹H NMR (400 MHz, DMSO-d6) δ 4.30 (d, J=5.65 Hz, 2H), 5.76 (t, J=5.65 Hz, 1H), 5.81 (s, 2H), 6.54 (d, J=2.01 Hz, 1H), 7.28 (d, J=2.13 Hz, 1H), 7.35 (d, J=1.13 Hz, 2H), 7.36 (s, 2H).

A solution of compound 14 (930 mg, 3.3 mmol, 1 eq) and 1,1,1-triethoxyethane (3.1 g, 19.1 mmol, 5.7 eq) in acetic acid (20 mL) was stirred at 130° C. for 3 h. The reaction mixture was concentrated in vacuo. The residue was redissolved in EtOAc (50 mL), washed with sat. aq. NaHCO₃ (2×50 mL), dried over anhydrous Na₂SO₄, filtered and concentrated to afford 15 (940 mg, 3.1 mmol, 93% yield) as a brown solid: ¹H NMR (400 MHz, DMSO-d6) δ 2.56 (s, 3H), 5.53 (s, 2H), 7.15 (d, J=6.90 Hz, 2H), 7.25-7.41 (m, 3H), 8.31 (d, J=2.13 Hz, 1H), 8.42 (d, J=2.13 Hz, 1H).

A mixture containing compound 15 (100 mg, 330.9 μmol, 1 eq), 4,4,5,5-tetramethyl-2-(4,4,5,5-tetramethyl-1,3,2-dioxaborolan-2-yl)-1,3,2-dioxaborolane (126.1 mg, 496.4 μmol, 1.5 eq), KOAc (64.9 mg, 661.9 μmol, 2 eq) and Pd(dppf)Cl₂ (24.2 mg, 33.1 μmol, 0.1 eq) in 1,4-dioxane (2.5 mL) was de-gassed and then heated at 90° C. for 12 h under nitrogen. The reaction mixture was cooled to 25° C., filtered and concentrated under vacuum. The residue was purified by column chromatography (6.5% MeOH in DCM) to afford 16 (130 mg, crude) as a brown solid ¹H NMR (400 MHz, CDCl₃) δ 1.37 (s, 12H), 2.63 (s, 3H), 5.39 (s, 2H), 7.29-7.40 (m, 5H), 8.00 (br. s., 1H), 8.92 (s, 1H).

A solution of compound 13 (130 mg, 286.3 μmol, 1 eq) and compound 16 (73.7 mg, 314.9 μmol, 1.1 eq) in 1, 4-dioxane (2 mL) and water (400 μt) was added Pd(PPh₃)₄ (33 mg, 28.6 μmol, 0.1 eq) and K₂CO₃ (79 mg, 572.6 μmol, 2 eq) in one portion under nitrogen. The reaction mixture was heated at 90° C. for 12 h. The reaction mixture was cooled to 25° C., filtered and concentrated in vacuum. The residue was purified by preparative HPLC to afford (4-(1-benzyl-2-methyl-1H-imidazo[4,5-b]pyridin-6-yl)-3-methylisoxazol-5-yl)methanol hydrochloride (Example 7) (10 mg, 30.8 umol, 11% yield) as a yellow solid: ¹H NMR (400 MHz, methanol-d₄) δ 2.26 (s, 3H), 3.01 (s, 3H), 4.63 (s, 2H), 5.80 (s, 2H), 7.38-7.48 (m, 5H), 8.31 (d, J=1.76 Hz, 1H), 8.74 (d, J=1.63 Hz, 1H). ESI m/z 335.2[M+1]⁺.

### Example 8: Preparation of 4-(1-Benzyl-2-methyl-1H-imidazo[4,5-b]pyridin-6-yl)-3-methylisoxazole

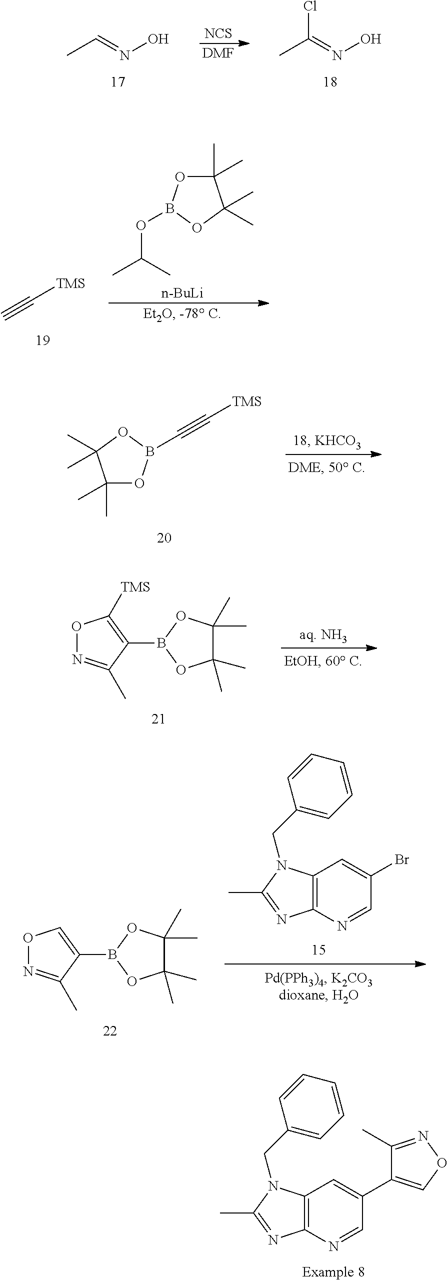

To a solution of compound 17 (1 g, 16.9 mmol, 1 eq) in DMF (15 mL) was added NCS (2.3 g, 17.3 mmol, 1 eq) in one portion at 25° C. under nitrogen. The mixture was heated at 50° C. for 2 h, cooled to room temperature, and poured into ice-water (w/w=1/1) (80 mL) and stirred for 1 min. The aqueous phase was extracted with EtOAc (3×30 mL). The combined organic fractions were washed with water (3×30 mL) and saturated brine (3×30 mL), dried over anhydrous Na₂SO₄, filtered and concentrated under vacuum to afford compound 18 (1.6 g, 16.9 mmol, 100% yield) as a yellow oil: ¹H NMR (400 MHz, CDCl₃) δ 2.29 (s, 3H), 8.06-8.16 (m, 1H).

To a solution of compound 19 (3 g, 30.5 mmol, 1.0 eq) in ethyl ether (30 mL) was added a solution of n-BuLi (2.5 M in hexane, 12.8 mL, 1.05 eq) dropwise at −78° C. over a period of 5 min under a nitrogen atmosphere. The reaction mixture was stirred at −78° C. for 1 h, then a solution of 2-isopropoxy-4,4,5,5-tetramethyl-1,3,2-dioxaborolane (5.7 g, 30.5 mmol, 1.0 eq) in THF (8 mL) was added dropwise. After stirring for 2 h at −78° C., the mixture was poured into cooled sat. aq. NH₄Cl (100 mL) and extracted with EtOAc (3×30 mL). The combined organic fractions were washed with saturated brine (3×30 mL), dried over anhydrous Na₂SO₄, filtered and concentrated in vacuo. The residue was dissolved in petroleum ether (30 mL) and cooled to −60° C. The resulting precipitate was collected by filtration and dried under vacuum to afford compound 20 (3.5 g, 15.6 mmol, 51% yield) as white solid: ¹H NMR (300 MHz, CDCl₃) δ0.21 (s, 9H) 1.30 (s, 12H).

A suspension of compound 20 (1 g, 4.5 mmol, 1.0 eq), compound 18 (421.2 mg, 4.5 mmol, 1.01 eq) and KHCO₃ (893.1 mg, 8.9 mmol, 2.00 eq) in DME (20 mL) was heated at 50° C. for 16 h. After cooling to room temperature, the reaction mixture was filtered and concentrated under vacuum. The residue was purified by column chromatography (0.5-2% EtOAc in PE) to afford compound 21 (1.3 g, 4.1 mmol, 92% yield, 91.9% purity) as a white solid: ¹H NMR (300 MHz, CDCl₃) δ 0.28-0.35 (s, 9H) 1.22-1.30 (s, 12H) 2.32 (s, 3H); ESI m/z 282.3[M+1]⁺.

A mixture of compound 21 (933.1 mg, 1.4 mmol, 1.00 eq) and 25% aqueous NH₃ (17 M, 5.0 mL, 59.8 eq) in ethanol (5 mL) was heated at 60° C. for 6 h. The reaction mixture was concentrated under vacuum to afford 22 (220 mg, crude) as a light yellow oil, which was used in the next step without purification. ESI m/z 210.2[M+1]⁺.

Compound 22 (52 mg, 248.2 umol, 1.5 eq), compound 15 (50 mg, 165.5 umol, 1.0 eq), Pd(PPh₃)₄ (19.1 mg, 16.6 umol, 0.1 eq) and K₂CO₃ (45.7 mg, 330.9 umol, 2 eq) in 1,4-dioxane (2.5 mL) and water (0.5 mL) was degassed and then heated at 95° C. for 10 h under a nitrogen atmosphere. After cooling to room temperature, the reaction mixture was filtered through a short plug of silica gel and the filtrate was concentrated to give a crude oil. The oil was purified by preparative HPLC, acidified with a drop of conc. HCl and lyophilized to afford 4-(1-benzyl-2-methyl-1H-imidazo[4,5-b]pyridin-6-yl)-3-methylisoxazole hydrochloride (Example 8) (14 mg, 45.6 umol, 28% yield, 99% purity) as a white solid: ¹H NMR (400 MHz, MeOH-d₄) δ 2.35 (s, 3H) 2.90-3.04 (m, 3H) 5.75-5.88 (m, 2H) 7.34-7.50 (m, 5H) 8.34 (d, J=1.76 Hz, 1H) 8.80 (d, J=1.63 Hz, 1H) 8.96-9.05 (m, 1H); ESI m/z 305.2[M+1]⁺.

### General Procedure B:

### Example 9: Preparation of 4-(3-((4,4-Difluoropiperidin-1-yl)methyl)-1H-indazol-5-yl)-3,5-dimethylisoxazole

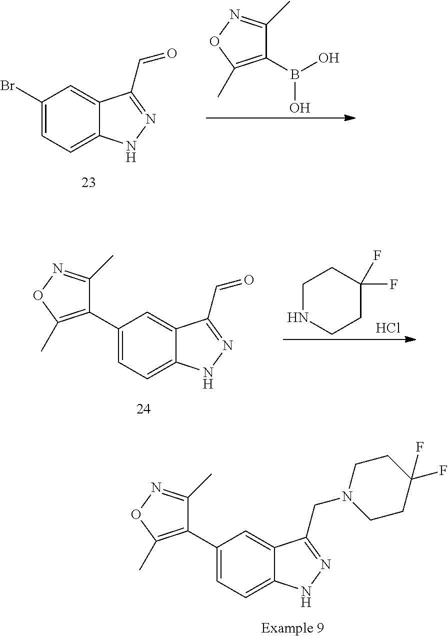

A mixture of compound 23 (500 mg, 2.22 mmol, 1.0 eq), (3,5-dimethylisoxazol-4-yl)boronic acid (469 mg, 3.33 mmol, 1.5 eq), Pd(dppf)Cl₂ (162 mg, 222 umol, 0.1 eq), K₂CO₃ (614 mg, 4.44 mmol, 2 eq) in dioxane (2 mL) and water (0.5 mL) was degassed and purged with nitrogen 3 times. Then the reaction mixture was stirred at 90° C. for 2 h under nitrogen atmosphere. TLC showed that the starting material was consumed completely, then the reaction cooled to room temperature. The solvent was removed under vacuum and the residue was suspended in THF (50 mL). The solid was removed by filtration and the filtrate was concentrated to give a brown solid. The solid was washed with petroleum ether (2×50 mL) and dried under vacuum to give compound 24 (300 mg, 1.24 mmol, 56% yield) as a brown solid: ¹H NMR (400 MHz, DMSO-d₆) δ 2.23 (s, 3H), 2.41 (s, 3H), 3.17 (s, 2H), 7.40 (d, J=8.16 Hz, 1H), 7.80 (d, J=8.66 Hz, 1H), 8.01 (s, 1H), 10.19 (s, 1H)

To a solution of compound 24 (100 mg, 414.52 umol, 1 eq) in THF (5 mL) and acetic acid (0.1 mL) was added 4,4-difluoropiperidine hydrochloride (65 mg, 0.81 eq). The mixture was stirred at 25° C. for 16 h, then NaBH(OAc)₃ (131 mg, 621 umol, 1.5 eq) was added. The mixture was stirred at 25° C. for 16 h at which time LC/MS showed that most of starting material was consumed. The reaction was quenched with MeOH (5 mL) and the solvent was removed under vacuum. The residue was purified by preparative HPLC and lyophilized to afford 4-(3-((4,4-difluoropiperidin-1-yl)methyl)-1H-indazol-5-yl)-3,5-dimethylisoxazole (Example 9) (15 mg, 10% yield) as a white solid: ¹H NMR (300 MHz, methanol-d4) δ 0.31-0.52 (m, 4H), 0.72 (s, 3H), 0.88 (s, 3H), 1.12 (br. s., 4H), 2.45 (s, 2H), 5.80 (d, J=8.67 Hz, 1H), 6.05 (d, J=8.85 Hz, 1H) 6.29 (s, 1H), ESI m/z 347.2[M+H]⁺.

### Example 10: Preparation of 3,5-Dimethyl-4-[3-(1-piperidylmethyl)-1H-indazol-5-yl]isoxazole

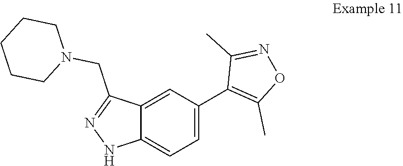 [SMILES: Cc1noc(C)c1-c1ccc2[nH]nc(CN3CCCCC3)c2c1]

Example 10 was synthesized according to General Procedure B substituting piperidine for 4,4-difluoropiperidine hydrochloride. 37 mg of white solid was isolated as a hydrochloride salt: ¹H NMR (400 MHz, Methanol-d₄) δ 1.44-1.61 (m, 1H), 1.71-1.89 (m, 3H), 1.97 (d, J=14.31 Hz, 2H), 2.30 (s, 3H), 2.46 (s, 3H), 3.13 (t, J=12.55 Hz, 2H), 3.67 (d, J=11.80 Hz, 2H), 4.76 (s, 2H), 7.45 (d, J=8.53 Hz, 1H), 7.73 (d, J=8.66 Hz, 1H), 7.94 (s, 1H); ESI m/z 311.2 [M+1]⁺.

### Example 11: Preparation of 3,5-Dimethyl-4-[3-[(4-methylpiperazin-1-yl)methyl]-1H-indazol-5-yl]isoxazole

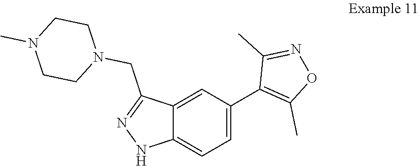 [SMILES: Cc1noc(C)c1-c1ccc2[nH]nc(CN3CCN(C)CC3)c2c1]

Example 11 was synthesized according to General Procedure B substituting 1-methylpiperazine for 4,4-difluoropiperidine hydrochloride. 27 mg of white solid was isolated as a hydrochloride salt: ¹H NMR (400 MHz, Methanol-d₄) δ 2.32 (s, 3H), 2.47 (s, 3H), 3.04 (s, 3H), 3.44-4.18 (m, 8H), 5.00 (s, 2H), 7.46 (d, J=8.53 Hz, 1H), 7.74 (d, J=8.66 Hz, 1H), 8.02 (s, 1H); ESI m/z 326.3 [M+1]⁺.

### Example 12: Preparation of 1-[[5-(3,5-Dimethylisoxazol-4-yl)-1H-indazol-3-yl]methyl]piperidine-4-carbonitrile

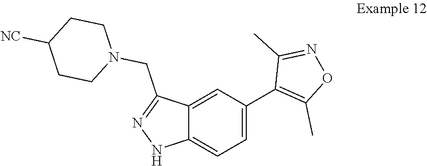 [SMILES: Cc1noc(C)c1-c1ccc2[nH]nc(CN3CCC(C#N)CC3)c2c1]

Example 12 was synthesized according to General Procedure B substituting piperidine-4-carbonitrile for 4,4-difluoropiperidine hydrochloride. 25 mg of a light yellow gum was isolated: ¹H NMR (400 MHz, CDCl₃) δ 1.81-2.04 (m, 4H), 2.31 (s, H), 2.38-2.58 (m, 5H), 2.61-2.90 (m, 3H), 3.97 (s, 2H), 7.31 (d, J=1.25 Hz, 1H), 7.56 (d, J=8.53 Hz, 1H), 7.75 (s, 1H), 10.25 (br s, 1H); ESI m/z 336.2 [M+1]⁺.

### Example 13: Preparation of 4-[3-[(4-Methoxy-1-piperidyl)methyl]-1H-indazol-5-yl]-3,5-dimethyl-isoxazole

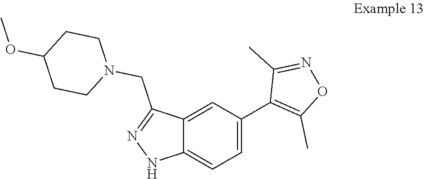 [SMILES: COC1CCN(Cc2n[nH]c3ccc(-c4c(C)noc4C)cc23)CC1]

Example 13 was synthesized according to General Procedure B substituting 4-methoxypiperidine for 4,4-difluoropiperidine hydrochloride. 27 mg of white solid was isolated as a hydrochloride salt: ¹H NMR (400 MHz, Methanol-d) δ 1.63-1.79 (m, 1H), 1.88-2.03 (m, 1H), 2.16 (d, 1=14.81 Hz, 1H), 2.23-2.36 (m, 4H), 2.46 (s, 3H), 3.15-3.30 (m, 1H), 3.34-3.41 (m, 4H), 3.43-3.80 (m, 3H), 4.70-4.83 (m, 2H), 7.44 (d, J=8.53 Hz, 1H), 7.73 (d, J=8.53 Hz, 1H), 7.94 (s, 1H); ESI m/z 341.0 [M+1]⁺.

### Example 14: Preparation of 4-[[S-(3,5-Dimethylisoxazol-4-yl)-1H-indazol-3-yl]methyl]morpholine

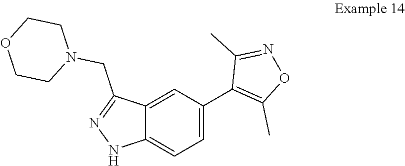 [SMILES: Cc1noc(C)c1-c1ccc2[nH]nc(CN3CCOCC3)c2c1]

Example 14 was synthesized according to General Procedure B substituting morpholine for 4,4-difluoropiperidine hydrochloride. 25 mg of yellow solid was isolated as a hydrochloride salt: ¹H NMR (300 MHz, Methanol-d₄) δ 7.86-7.91 (m, 1H), 7.69-7.76 (m, 1H), 7.41-7.48 (m, 1H), 4.79-4.83 (m, 2H), 4.07 (d, J=11.1 Hz, 2H), 3.75 (t, J=12.2 Hz, 2H), 3.59 (d, J=12.4 Hz, 2H), 3.39 (br. s., 2H), 2.44 (s, 3H), 2.28 ppm (s, 3H); ESI m/z 313.0[M+1]⁺.

### Example 15: Inhibition of tetra-acetylated histoneH4 binding individual BET Bromodomains

Proteins were cloned and overexpressed with a N-terminal 6×His tag, then purified by nickel affinity followed by size exclusion chromatography. Briefly, E. coli BL21(DE3) cells were transformed with a recombinant expression vector encoding N-terminally Nickel affinity tagged bromodomains from Brd2, Brd3, Brd4. Cell cultures were incubated at 37° C. with shaking to the appropriate density and induced overnight with IPTG. The supernatant of lysed cells was loaded onto Ni-IDA column for purification. Eluted protein was pooled, concentrated and further purified by size exclusion chromatography. Fractions representing monomeric protein were pooled, concentrated, aliquoted, and frozen at −80° C. for use in subsequent experiments.

Binding of tetra-acetylated histone H4 peptide (Millipore) and BET bromodomains was confirmed by Amplified Luminescent Proximity Homogenous Assay (AlphaScreen). N-terminally His-tagged bromodomains (BRD4(1) at 20 nM and BRD4(2) at 100 nM) and biotinylated tetra-acetylated histone H4 (10-25 nM) were incubated in the presence of nickel chelate acceptor beads and streptavidin donor beads (PerkinAlmer, 6760000K) added to a final concentration of 2 μg/ml under green light in a white 96 well microtiter plate (Greiner). For inhibition assays, serially diluted compounds were added to the reaction mixtures in a 0.1% final concentrations of DMSO. Final buffer concentrations were 50 mM HEPES, 100 mM NaCl and 0.1% BSA buffer, pH 7.4 and optimized to 30 min incubation time. Assay plates were read at 570 nM on a Synergy H4 Plate Reader (Biotek). IC₅₀ values were determined from a dose response curve.

Compounds with an IC₅₀ value less than or equal to 0.3 μM were deemed to be highly active (+++); compounds with an IC₅₀ value between 0.3 and 3 μM were deemed to be very active (++); compounds with an IC₅₀ value between 3 and 30 μM were deemed to be active (+).

TABLE 2
Inhibition of Tetra-acetylated Histone H4 Binding to Brd4
bromodomain 1 (BRD4(1) as Measured by AlphaScreen

| | AlphaScreen | | | | AlphaScreen | | |
|---|---|---|---|---|---|---|---|
| Example | activity | Example | AlphaScreen | Example | activity | Example | AlphaScreen |
| Number | BRD4(1) | Number | activityBRD4(1) | Number | BRD4(1) | Number | activityBRD4(1) |
| 1 | +++ | 2 | +++ | 3 | +++ | 4 | +++ |
| 5 | +++ | 6 | + | 7 | +++ | 8 | +++ |
| 9 | +++ | 10 | +++ | 11 | ++ | 12 | +++ |
| 13 | ++ | 14 | ++ | — | — | — | — |

### Example 16: Inhibition of cMYC Expression in Cancer Cell Lines

MV4-11 cells (CRL-9591) are plated at a density of 2.5×10′ cells per well in 96 well U-bottom plates and are treated with increasing concentrations of test compound or DMSO (0.1%) in IMDM media containing 10% FBS and penicillin/streptomycin, and are incubated for 3 h at 37° C. Triplicate wells are used for each concentration. Cells are pelleted by centrifugation and are harvested using the mRNA Catcher PLUS kit according to manufacturer's instructions. The eluted mRNA isolated is then used in a one-step quantitative real-time PCR reaction, using components of the RNA UltraSense™ One-Step Kit (Life Technologies) together with Applied Biosystems TaqMan® primer-probes for cMYC and Cyclophilin. Real-time PCR plates are run on a ViiA™7 real time PCR machine (Applied Biosystems). The data is analyzed, and the Ct values for cMYC are normalized to an internal control prior to determining the fold expression of each sample, relative to the control.

Compounds with an IC₅₀ value less than or equal to 0.3 μM are deemed to be highly active (+++); compounds with an IC₅₀ value between 0.3 and 3 μM are deemed to be very active (++); compounds with an IC₅₀ value between 3 and 30 μM are deemed to be active (+).

### Example 17: Inhibition of Cell Proliferation in Cancer Cell Lines

MV4-11 cells (CRL-9591) are plated at a density of 5×10⁴ cells per well in 96 well flat bottom plates and treated with increasing concentrations of test compound or DMSO (0.1%) in IMDM media containing 10% FBS and penicillin/streptomycin. Triplicate wells are used for each concentration and a well containing only media was used as a control. Plates are incubated at 37° C., 5% CO₂ for 72 h before adding 20 μL of the CellTiter Aqueous One Solution (Promega) to each well and are incubated at 37° C., 5% CO₂ for an additional 3-4 h. The absorbance is read at 490 nm in a spectrophotometer and the percentage of cell titer relative to DMSO-treated cells is calculated after correcting for background by subtracting the blank well's signal. IC₅₀ values are calculated using the GraphPad Prism software.

Compounds with an IC₅₀ value less than or equal to 0.3 μM are deemed to be highly active (+++); compounds with an IC₅₀ value between 0.3 and 3 μM are deemed to be very active (++); compounds with an IC₅₀ value between 3 and 30 μM are deemed to be active (+).

### Example 18: Inhibition of hIL-6 mRNA Transcription

Human leukemic monocyte lymphoma U937 cells (CRL-1593.2) were plated at a density of 3.2×104 cells per well in a 96-well plate in 100 μL RPMI-1640 containing 10% FBS and penicillin/streptomycin, and were differentiated into macrophages for 3 days in 60 ng/mL PMA (phorbol-13-myristate-12-acetate) at 37° C. in 5% CO2 prior to the addition of compound. The cells were pretreated for 1 h with increasing concentrations of test compound in 0.1% DMSO prior to stimulation with 1 ug/mL lipopolysaccharide from Escherichia coli. Triplicate wells were used for each concentration. The cells were incubated at 37′C, 5% CO₂ for 3 h before the cells were harvested. At time of harvest, media was removed and cells were rinsed in 200 μL PBS. Cells were harvested using the mRNA Catcher PLUS kit according to manufacturer's instructions. The eluted mRNA was then used in a one-step quantitative real-time PCR reaction using components of the RNA UltraSense™ One-Step Kit (Life Technologies) together with Applied Biosystems TaqMan® primer-probes for hIL-6 and Cyclophilin. Real-time PCR plates were run on a ViiA™7 real time PCR machine (Applied Biosystems). The data was analyzed, and the Ct values for hIL-6 were normalized to an internal control prior to determining the fold expression of each sample, relative to the control.

Compounds with an IC₅₀ value less than or equal to 0.3 μM were deemed to be highly active (+++); compounds with an IC₅₀ value between 0.3 and 3 μM were deemed to be very active (++); compounds with an IC₅₀ value between 3 and 30 μM were deemed to be active (+).

TABLE 3
Inhibition of hIL-6 mRNA Transcription

| Example | IL-6 | Example | IL-6 | Example | IL-6 | Example | IL-6 |
|---|---|---|---|---|---|---|---|
| Number | activity | Number | activity | Number | activity | Number | activity |
| 1 | ++ | 2 | ++ | 3 | ++ | 4 | ++ |
| 5 | not | 6 | not | 7 | ++ | 8 | ++ |
| | active | | active | | | | |
| 9 | ++ | 10 | ++ | 11 | + | 12 | ++ |
| 13 | ++ | 14 | + | — | — | — | — |

### Example 19: Inhibition of hIL-17 mRNA Transcription

Human peripheral blood mononuclear cells were plated (2.0×10⁵ cells per well) in a 96-well plate in 45 μL OpTimizer T Cell expansion media (Life Technologies) containing 20 ng/ml IL-2 and penicillin/streptomycin. The cells were treated with increasing concentrations of the test compound or DMSO (0.1%), and were incubated at 37° C., 5% CO₂ for 1 h before addition of 10× stock OKT3 antibody at 10 ug/ml in media. Triplicate wells were used for each concentration. Cells were incubated at 37° C., 5% CO₂ for 6 h before the cells were harvested. At time of harvest, cells were pelleted by centrifugation at 800 rpm for 5 min. Cells were harvested using the mRNA Catcher PLUS kit according to manufacturer's instructions. The eluted mRNA was then used in a one-step quantitative real-time PCR reaction, using components of the RNA UltraSense™ One-Step Kit (Life Technologies) together with Applied Biosystems TaqMan® primer-probes for hIL-17 and Cyclophilin. Real-time PCR plates were run on a ViiA™7 real time PCR machine (Applied Biosystems). The data was analyzed, and the Ct values for hIL-17 were normalized to an internal control prior to determining the fold induction of each unknown sample, relative to the control.

Compounds with an IC₅₀ value less than or equal to 0.3 μM were deemed to be highly active (+++); compounds with an IC₅₀ value between 0.3 and 3 μM were deemed to be very active (++); compounds with an IC₅₀ value between 3 and 30 μM were deemed to be active (+).

TABLE 4
Inhibition of hIL-17 mRNA Transcription

| Example | IL-7 | Example | IL-7 | Example | IL-17 | Example | IL-7 |
|---|---|---|---|---|---|---|---|
| Number | activity | Number | activity | Number | activity | Number | activity |
| 1 | ++ | 2 | ++ | 4 | + | 5 | Not |
| | | | | | | | active |
| 6 | Not | 9 | ++ | 10 | ++ | 11 | ++ |
| | active | | | | | | |

### Example 20: Inhibition of hVCAM mRNA Transcription

Human umbilical vein endothelial cells (HUVECs) are plated in a 96-well plate (4.0×10³ cells per well) in 100 μL EGM media and incubated for 24 h prior to the addition of increasing concentrations of the compound of interest or DMSO (0.1%). Triplicate wells are used for each concentration. The cells are pretreated for 1 h with the test compound prior to stimulation with tumor necrosis factor-α when they are incubated for an additional 24 h before the cells are harvested. At time of harvest, the spent media is removed and HUVECs are rinsed in 200 μL PBS. Cells are harvested using the mRNA Catcher PLUS kit according to manufacturer's instructions. The eluted mRNA is then used in a one-step quantitative real-time PCR reaction, using components of the RNA UltraSense™ One-Step Kit (Life Technologies) together with Applied Biosystems TaqMan® primer-probes for hVCAM and Cyclophilin. Real-time PCR plates are run on a ViiA™7 real time PCR machine (Applied Biosystems). The data is analyzed, and the Ct values for cMYC are normalized to an internal control prior to determining the fold expression of each sample, relative to the control.

### Example 21: Inhibition of hMCP-1 mRNA Transcription

Human Peripheral Blood Mononuclear Cells are plated at a density of 1.0×10⁵ cells per well in a 96-well plate in RPMI-1640 containing 10% FBS and penicillin/streptomycin. The cells are treated with increasing concentrations of the compound or DMSO (0.1%), and are incubated at 37° C., 5% CO2 for 3 h before the cells are harvested. At time of harvest, cells are transferred to V-bottom plates and pelleted by centrifugation at 800 rpm for 5 min. Cells are harvested using the mRNA Catcher PLUS kit according to manufacturer's instructions. The eluted mRNA is then used in a one-step quantitative real-time PCR reaction, using components of the RNA UltraSense™ One-Step Kit (Life Technologies) together with Applied Biosystems TaqMan® primer-probes for hMCP-1 and Cyclophilin. Real-time PCR plates are run on a ViiA™7 real time PCR machine (Applied Biosystems). The data is analyzed, and the Ct values for cMYC are normalized to an internal control prior to determining the fold expression of each sample, relative to the control.

### Example 22: Up-regulation of hApoA-1 mRNA Transcription

In this example, hApoA-I mRNA in tissue culture cells is quantitated to measure the transcriptional up-regulation of hApoA-I when treated with a compound of the present disclosure. Huh7 cells (2.5×10⁵ per well) are plated in a 96-well plate using 100 μL DMEM per well, (Gibco DMEM supplemented with penicillin/streptomycin and 10% FBS), 72 h before the addition of the compound. The cells are treated with increasing concentrations of the compound or DMSO (0.1%), and incubated at 37° C., 5% CO2 for 48 h. Spent media is removed from the Huh-7 cells and placed on ice for immediate use with the “LDH cytotoxicity assay Kit II” from Abcam. The cells remaining in the plate are rinsed with 100 μL PBS. Cells were harvested using the mRNA Catcher PLUS kit according to manufacturer's instructions. The eluted mRNA is then used in a one-step quantitative real-time PCR reaction, using components of the RNA UltraSense™ One-Step Kit (Life Technologies) together with Applied Biosystems TaqMan® primer-probes for hApoA-I and Cyclophilin. Real-time PCR plates are run on a ViiA™7 real time PCR machine (Applied Biosystems). The data is analyzed, and the Ct values for cMYC are normalized to an internal control prior to determining the fold expression of each sample, relative to the control.

Compounds with an EC₁₇₀ value less than or equal to 0.3 μM are deemed to be highly active (+++); compounds with an EC₁₇₀ value between 0.3 and 3 μM are deemed to be very active (++); compounds with an EC₁₇₀ value between 3 and 30 μM are deemed to be active (+).

### Examples 23: In Vivo Efficacy in Athymic Nude Mouse Strain of an Acute Myeloid Leukemia Xenograft Model Using MV4-11 Cells

MV4-11 cells (ATCC) are grown under standard cell culture conditions and (NCr) nu/nu fisol strain of female mice age 6-7 weeks are injected with 5×10⁶ cells/animal in 100 μL PBS+100 μL Matrigel in the lower left abdominal flank. By approximately day 18-21 after MV4-11 cells injection, mice are randomized based on tumor volume (L×W×H)/2) of average ˜100-300 mm³. Mice are dosed orally with compound at 5 to 120 mg/kg b.i.d and/or q.d. on a continuous dosing schedule and at 2.5 to 85 mg/kg q.d. on a 5 day on 2 day off, 100 mg/kg q.d. on a 4 day on and 3 day off, 135 mg/kg q.d. on a 3 day on and 4 day off, 180 mg/kg on a 2 day on and 5 day off and 240 mg/kg on a 1 day on and 6 days off dosing schedules in EA006 formulation at 10 mL/kg body weight dose volume. Tumor measurements are taken with electronic micro calipers and body weights measured on alternate days beginning from dosing period. The average tumor volumes, percent Tumor Growth Inhibition (TGI) and % change in body weights are compared relative to Vehicle control animals. The means, statistical analysis and the comparison between groups are calculated using Student's t-test in Excel.

### Example 24: In Vivo Efficacy in Athymic Nude Mouse Strain of an Acute Myeloid Leukemia Xenograft Model Using OCI-3 AML Cells

OCI-3 AML cells (DMSZ) are grown under standard cell culture conditions and (NCr) nu/nu fisol strain of female mice age 6-7 weeks are injected with 10×0 cells/animal in 100 μL PBS+100 μL Matrigel in the lower left abdominal flank. By approximately day 18-21 after OCI-3 AML cells injection, mice are randomized based on tumor volume (L×W×H)/2) of average ˜100-300 mm³. Mice are dosed orally with compound at 30 mg/kg b.i.d on a continuous dosing schedule and at 2.5 to 45 mg/kg q.d. on a 5 day on and 2 day off dosing schedule in EA006 formulation at 10 ml/kg body weight dose volume. Tumor measurements are taken with electronic micro calipers and body weights measured on alternate days beginning from dosing period. The average tumor volumes, percent Tumor Growth Inhibition (TGI) and % change in body weights are compared relative to Vehicle control animals. The means, statistical analysis and the comparison between groups are calculated using Student's t-test in Excel.

### Example 25: Evaluation of Target Engagement

MV4-11 and MM1.s cells (ATCC) are grown under standard cell culture conditions and (NCr) nu/nu fisol strain of female mice age 6-7 weeks are injected with 5×10⁶ cells/animal in 100 μL PBS+100 μL Matrigel in the lower left abdominal flank. By approximately day 28 after MV4-11 and MM1.s cells injection, mice are randomized based on tumor volume (L×W×H)/2) of average ˜500 mm³. Mice are dosed orally with compound in EA006 formulation at 10 mL/kg body weight dose volume and tumors harvested 3, 6, 12, 24 hrs post dose for Bcl2 and c-myc gene expression analysis as PD biomarkers.

### Example 26: In Vivo Efficacy in Mouse Endotoxemia Model Assay

Sub lethal doses of Endotoxin (E. Coli bacterial lipopolysaccharide) are administered to animals to produce a generalized inflammatory response which is monitored by increases in secreted cytokines. Compounds are administered to C57/BI16 mice at T=4 hours orally at 75 mg/kg dose to evaluate inhibition in IL-6 and IL-17 and MCP-1 cytokines post 3-h challenge with lipopolysaccharide (LPS) at T=0 hours at 0.5 mg/kg dose intraperitoneally.

### Example 27: In Vivo Efficacy in Rat Collagen-Induced Arthritis

Rat collagen-induced arthritis is an experimental model of polyarthritis that has been widely used for preclinical testing of numerous anti-arthritic agents. Following administration of collagen, this model establishes a measurable polyarticular inflammation, marked cartilage destruction in association with pannus formation and mild to moderate bone resorption and periosteal bone proliferation. In this model, collagen are administered to female Lewis strain of rats on Day 1 and 7 of study and dosed with compounds from Day 11 to Day 17. Test compounds are administered at 25 mg/kg to 120 mg/kg b.i.d and 7.5 mg/kg to 30 mg/kg q.d dose to assess the potential to inhibit the inflammation (including paw swelling), cartilage destruction and bone resorption in arthritic rats, using a model in which the treatment is administered after the disease has been established.

### Example 28: In Vivo Efficacy in Experimental Autoimmune Encephalomyelitis (EAE) Model of MS

Experimental autoimmune encephalomyelitis (EAE) is a T-cell-mediated autoimmune disease of the CNS which shares many clinical and histopathological features with human multiple sclerosis (MS). EAE is the most commonly used animal model of MS. T cells of both Th1 and Th17 lineage have been shown to induce EAE. Cytokines IL-23, IL-6 and IL-17, which are either critical for Th1 and Th17 differentiation or produced by these T cells, play a critical and non-redundant role in EAE development. Therefore, drugs targeting production of these cytokines are likely to have therapeutic potential in treatment of MS.

Compounds of Formula I (including Formula Ia and Formula Ib) are administered at 50 to 125 mg/kg b.i.d. from time of immunization to EAE mice to assess anti-inflammatory activity. In this model, EAE is induced by MOG₃₅₋₅₅/CFA immunization and pertussis toxin injection in female C57BI/6 mice.

### Example 29: Ex Vivo Effects on T Cell Function from Splenocyte and Lymphocyte Cultures Stimulated with External MOG Stimulation

Mice are immunized with MOG/CFA and simultaneously treated with the compound for 11 days on a b.i.d regimen. Inguinal Lymph node and spleen are harvested, cultures are set up for lymphocytes and splenocytes and stimulated with external antigen (MOG) for 72 hours. Supernatants from these cultures are analyzed for TH1, Th2 and Th17 cytokines using a Cytometric Bead Array assay.

### Example 30: In Vivo Efficacy in Athymic Nude Mouse Strain of Multiple Myeloma Xenograft Model Using MM1.s Cells

MM1.s cells (ATCC) are grown under standard cell culture conditions and SCID-Beige strain of female mice age 6-7 weeks are injected with 10×10⁶ cells/animal in 100 μL PBS+100 μL Matrigel in the lower left abdominal flank. By approximately day 21 after MM1.s cells injection, mice are randomized based on tumor volume (L×W×H)/2) of average ˜120 mm³. Mice are dosed orally with compound at 25 to 90 mg/kg b.i.d and or q.d in EA006 formulation at 10 mL/kg body weight dose volume. Tumor measurements are taken with electronic micro calipers and body weights measured on alternate days beginning from dosing period. The average tumor volumes, percent Tumor Growth Inhibition (TGI) and % change in body weights are compared relative to Vehicle control animals. The means, statistical analysis and the comparison between groups are calculated using Student's t-test in Excel.

Other embodiments of the present disclosure will be apparent to those skilled in the art from consideration of the specification and practice of the present disclosure disclosed herein. It is intended that the specification and examples be considered as exemplary only, with a true scope and spirit of the present disclosure being indicated by the following claims.

1-63. (canceled)

64. A compound of Formula I: 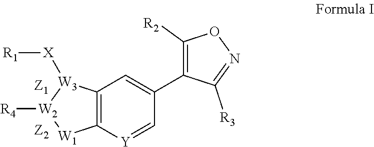
or a stereoisomer, tautomer, pharmaceutically acceptable salt, or hydrate thereof,
wherein:
W₁ is selected from N and NH;
W₂, and W₃ are selected from C and N;
Z₁ and Z₂ are selected from a single bond and a double bond;
R₁ is selected from carbocycle (C₅-C₁₀) and heterocycle (C₂-C₁₀) optionally substituted with 1 to 5 groups independently selected from R₅;
R₂ is selected from hydrogen and alkyl (C₁-C₆) optionally substituted with halogen and/or hydroxyl;
R₃ is selected from alkyl (C₁-C₆) optionally substituted with halogen and/or hydroxyl, with the proviso that if R₂ and R₃ are methyls, then R₁ is different from: 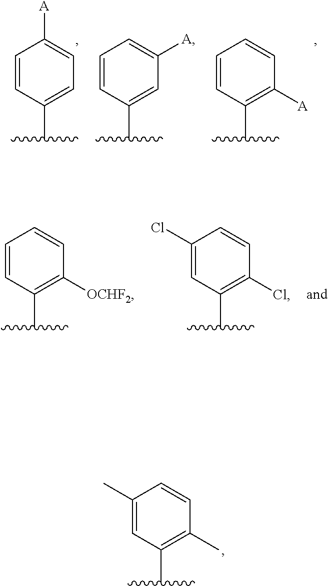
wherein A is selected from hydrogen, halogen, methoxy, —CN, —NO₂, —COOMe, and —CONMe₂;
R₄ if present, is selected from hydrogen, alkyl (C₁-C₁₀), carbocycle (C₃-C₁₀), and heterocycle (C₂-C₁₀) optionally substituted with 1 to 5 groups independently selected from R₅;
each R₅ is independently selected from deuterium, alkyl(C₁-C₆), alkoxy(C₁-C₆), amino, —NHC(O)NH-alkyl(C₁-C₆), halogen, amide, —CF₃, —CN, —N₃, ketone (C₁-C₆), —S(O)-alkyl(C₁-C₄), —SO₂-alkyl(C₁-C₆), thioalkyl(C₁-C₆), —COOH, and ester, each of which may be optionally substituted with hydrogen, F, Cl, Br, —OH, —NH₂, —NHMe, —OMe, —SMe, oxo, and/or thio-oxo;
X is selected from —CH₂— optionally substituted with 1 to 2 groups independently selected from R₅;
Y is selected from N and CH;
wherein if W₃ is C, then W₂ is N, W₁ is NH, Z₁ is a double bond, Z₂ is a single bond, and R₄ is absent; and
wherein if W₃ is N, then W₂ is C, W₁ is N, Z₁ is a single bond and Z₂ is a double bond.

65. The compound according to claim 64, wherein the compound is a compound of Formula Ia: 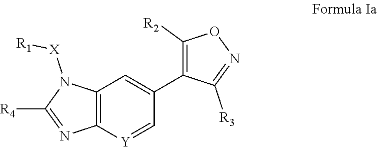
or a stereoisomer, tautomer, pharmaceutically acceptable salt, or hydrate thereof,
wherein:
R₁ is selected from carbocycle (C₅-C₁₀) and heterocycle (C₂-C₁₀) optionally substituted with 1 to 5 groups independently selected from R₅;
R₂ is selected from hydrogen and alkyl (C₁-C₆) optionally substituted with halogen and/or hydroxyl;
R₃ is selected from alkyl (C₁-C₆) optionally substituted with halogen and/or hydroxyl, with the proviso that if R₂ and R₃ are methyls, then R₁ is different from: 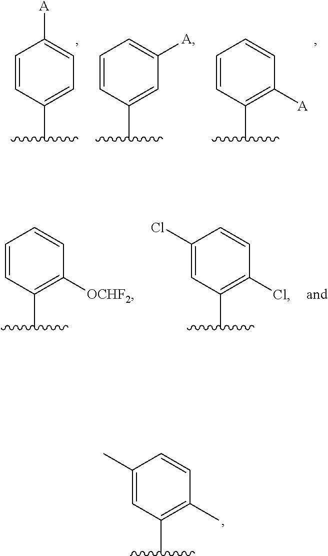
wherein A is selected from hydrogen, halogen, methoxy, —CN, —NO₂, —COOMe, and —CONMe₂;
R₄ is selected from hydrogen, alkyl (C₁-C₁₀), carbocycle (C₃-C₁₀), and heterocycle (C₂-C₁₀) optionally substituted with 1 to 5 groups independently selected from R₅;
each R₅ is independently selected from deuterium, alkyl(C₁-C₆), alkoxy(C₁-C₆), amino, —NHC(O)NH-alkyl(C₁-C₆), halogen, amide, —CF₃, —CN, —N₃, ketone (C₁-C₆), —S(O)-alkyl(C₁-C₄), —SO₂-alkyl(C₁-C₆), thioalkyl(C₁-C₆), —COOH, and ester, each of which may be optionally substituted with hydrogen, F, Cl, Br, —OH, —NH₂, —NHMe, —OMe, —SMe, oxo, and/or thio-oxo;
X is selected from —CH₂— optionally substituted with 1 to 2 groups independently selected from R₅; and
Y is selected from N and CH.

66. The compound according to claim 64, wherein the compound is a compound of Formula Ib: 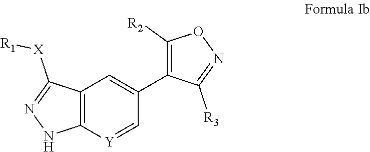
or a stereoisomer, tautomer, pharmaceutically acceptable salt, or hydrate thereof,
wherein:
R₁ is selected from carbocycle (C₅-C₁₀) and heterocycle (C₂-C₁₀) optionally substituted with 1 to 5 groups independently selected from R₅;
R₂ is selected from hydrogen and alkyl (C₁-C₆) optionally substituted with halogen and/or hydroxyl;
R₃ is selected from alkyl (C₁-C₆) optionally substituted with halogen and/or hydroxyl, with the proviso that if R₂ and R₃ are methyls, then R₁ is different from: 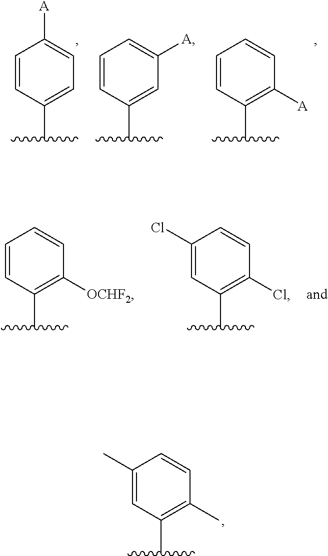
wherein A is selected from hydrogen, halogen, methoxy, —CN, —NO₂, —COOMe, and —CONMe₂;
each R₅ is independently selected from deuterium, alkyl(C₁-C₆), alkoxy(C₁-C₆), amino, —NHC(O)NH-alkyl(C₁-C₆), halogen, amide, —CF₃, —CN, —N₃, ketone (C₁-C₆), —S(O)-alkyl(C₁-C₄), —SO₂-alkyl(C₁-C₆), thioalkyl(C₁-C₆), —COOH, and ester, each of which may be optionally substituted with hydrogen, F, Cl, Br, —OH, —NH₂, —NHMe, —OMe, —SMe, oxo, and/or thio-oxo;
X is selected from —CH₂— optionally substituted with 1 to 2 groups independently selected from R₅; and
Y is selected from N and CH.

67. The compound according to claim 64, wherein R₁ is selected from phenyl groups optionally substituted with 1 to 5 groups independently selected from R₅.

68. The compound according to claim 67, wherein R₁ is selected from phenyl groups substituted with 1 to 5 groups independently selected from thioalkyl(C₁-C₆), ester, —S(O)-alkyl(C₁-C₄), and —COOH.

69. The compound according claim 67, wherein R₁ is unsubstituted phenyl.

70. The compound according to claim 64, wherein R₁ is selected from: 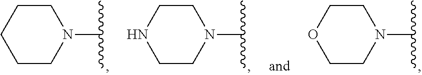
each of which is optionally substituted with 1 to 5 groups independently selected from R₅.

71. The compound according to claim 70, wherein R₁ is selected from: 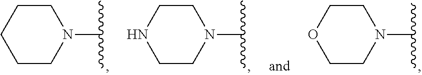
each of which is optionally substituted with 1 to 5 groups independently selected from —CN, alkyl(C₁-C₆), alkoxy(C₁-C₆), and halogen.

72. The compound according to claim 64, wherein R₂ and R₃ are methyl groups independently and optionally substituted with halogen and/or hydroxyl.

73. The compound according to claim 72, wherein R₂ and R₃ are methyl groups.

74. The compound according to claim 64, wherein R₄ is selected from alkyl (C₁-C₆) optionally substituted with 1 to 5 groups independently selected from R₅.

75. The compound according to claim 74, wherein R₄ is methyl.

76. The compound according to claim 64, wherein Y is CH.

77. The compound according to claim 64, wherein Y is N.

78. The compound according to claim 64, wherein X is CH₂.

79. The compound according to claim 64, wherein the compound is selected from:
3,5-Dimethyl-4-(2-methyl-1-(4-(methylthio)benzyl)-1H-imidazo[4,5-b]pyridin-6-yl)isoxazole;
Methyl 3-((6-(3,5-dimethylisoxazol-4-yl)-2-methyl-1H-imidazo[4,5-b]pyridin-1-yl)methyl)benzoate;
Methyl 4-((6-(3,5-dimethylisoxazol-4-yl)-2-methyl-1H-imidazo[4,5-b]pyridin-1-yl)methyl)benzoate;
3,5-Dimethyl-4-(2-methyl-1-(3-(methylthio)benzyl)-1H-imidazo[4,5-b]pyridin-6-yl)isoxazole;
3,5-Dimethyl-4-(2-methyl-1-(4-(methylsulfinyl)benzyl)-1H-imidazo[4,5-b]pyridin-6-yl)isoxazole;
3-((6-(3,5-Dimethylisoxazol-4-yl)-2-methyl-1H-imidazo[4,5-b]pyridin-1-yl)methyl)benzoic acid;
(4-(1-Benzyl-2-methyl-1H-imidazo[4,5-b]pyridin-6-yl)-3-methylisoxazol-5-yl)methanol;
4-(1-Benzyl-2-methyl-1H-imidazo[4,5-b]pyridin-6-yl)-3-methylisoxazole;
4-(3-((4,4-Difluoropiperidin-1-yl)methyl)-1H-indazol-5-yl)-3,5-dimethylisoxazole;
3,5-Dimethyl-4-[3-(1-piperidylmethyl)-1H-indazol-5-yl]isoxazole;
3,5-Dimethyl-4-[3-[(4-methylpiperazin-1-yl)methyl]-1H-indazol-5-yl]isoxazole;
1-[[5-(3,5-Dim ethyl isoxazol-4-yl)-1H-indazol-3-yl]methyl]piperidine-4-carbonitrile;
4-[3-[(4-Methoxy-1-piperidyl)methyl]-1H-indazol-5-yl]-3,5-dimethyl-isoxazole;
4-[[5-(3,5-Dimethylisoxazol-4-yl)-1H-indazol-3-yl]methyl]morpholine;
and stereoisomers, tautomers, pharmaceutically acceptable salts, or hydrates thereof.

80. A pharmaceutical composition comprising the compound of claim 64, and a pharmaceutically acceptable carrier.

81. A pharmaceutical composition comprising the compound of claim 79, and a pharmaceutically acceptable carrier.

82. A method for inhibition of BET protein function comprising administering a therapeutically effective amount of the compound of claim 64.

83. A method of treating a disease or disorder selected from:
autoimmune diseases or disorders;
inflammatory diseases or disorders;
acute non-autoimmune inflammatory diseases or disorders; and
chronic non-autoimmune inflammatory diseases or disorders;
comprising administering a therapeutically effective amount of the compound of claim 64.

84. The method of claim 83, wherein:
the autoimmune or inflammatory disease or disorder is selected from Acute Disseminated Encephalomyelitis, Agammaglobulinemia, Allergic Disease, Ankylosing spondylitis, Anti-GBM/Anti-TBM nephritis, Anti-phospholipid syndrome, Autoimmune aplastic anemia, Autoimmune hepatitis, Autoimmune inner ear disease, Autoimmune myocarditis, Autoimmune pancreatitis, Autoimmune retinopathy, Autoimmune thrombocytopenic purpura, Behcet's Disease, Bullous pemphigoid, Castleman's Disease, Celiac Disease, Churg-Strauss syndrome, Crohn's Disease, Cogan's syndrome, Dry eye syndrome, Essential mixed cryoglobulinemia, Dermatomyositis, Devic's Disease, Encephalitis, Eosinophlic esophagitis, Eosinophilic fasciitis, Erythema nodosum, Giant cell arteritis, Glomerulonephritis, Goodpasture's syndrome, Granulomatosis with Polyangiitis (Wegener's), Graves' Disease, Guillain-Barre syndrome, Hashimoto's thyroiditis, Hemolytic anemia, Henoch-Schonlein purpura, idiopathic pulmonary fibrosis, IgA nephropathy, Inclusion body myositis, Type I diabetes, Interstitial cystitis, Kawasaki's Disease, Leukocytoclastic vasculitis, Lichen planus, Lupus (SLE), Microscopic polyangitis, Multiple sclerosis, Myasthenia gravis, myositis, Optic neuritis, Pemphigus, POEMS syndrome, Polyarteritis nodosa, Primary biliary cirrhosis, Psoriasis, Psoriatic arthritis, Pyoderma gangrenosum, Relapsing polychondritis, Rheumatoid arthritis, Sarcoidosis, Scleroderma, Sjogren's syndrome, Takayasu's arteritis, Transverse myelitis, Ulcerative colitis, Uveitis, and Vitiligo; and
the acute or chronic non-autoimmune inflammatory disease or disorder is selected from sinusitis, pneumonitis, osteomyelitis, gastritis, enteritis, gingivitis, appendicitis, irritable bowel syndrome, tissue graft rejection, chronic obstructive pulmonary disease (COPD), septic shock, osteoarthritis, acute gout, acute lung injury, acute renal failure, burns, Herxheimer reaction, and SIRS associated with viral infections.

85. A method of treating cancer comprising administering a therapeutically effective amount of the compound according to claim 64.

86. The method of claim 85, wherein the cancer is selected from Burkitt's lymphoma, Hodgkin's lymphoma, non-Hodgkin's lymphoma, diffuse large B-cell lymphoma, follicular lymphoma, multiple myeloma, bladder cancer, breast cancer, colon cancer, melanoma, ovarian cancer, prostate cancer, small cell lung carcinoma, non-small cell lung cancer, NUT midline carcinoma, acute B-cell lymphoma, and head and neck squamous cell carcinoma.

87. The method of claim 85, wherein the cancer is selected from:
a cancer associated with overexpression, translocation, amplification, or rearrangement of a myc family oncoprotein that is sensitive to BET inhibition;
a cancer associated with overexpression, translocation, amplification, or rearrangement of BET proteins;
a cancer that relies on pTEFb (Cdk9/cyclin T) and BET proteins to regulate oncogenes;
a cancer associated with upregulation of BET responsive genes selected from CDK6, Bcl2, TYRO3, MYB, and hTERT;
a cancer that is sensitive to effects of BET inhibition;
a cancer associated with a virus; and
a cancer associated with a gene regulated by a super enhancer.

88. The method of claim 87, wherein:
the cancer associated with overexpression, translocation, amplification, or rearrangement of a myc family oncoprotein is selected from B-acute lymphocytic leukemia, Burkitt's lymphoma, Diffuse large B-cell lymphoma, Multiple myeloma, Primary plasma cell leukemia, Atypical carcinoid lung cancer, Bladder cancer, Breast cancer, Cervix cancer, Colon cancer, Gastric cancer, Glioblastoma, Hepatocellular carcinoma, Large cell neuroendocrine carcinoma, Medulloblastoma, Melanoma, nodular, Melanoma, superficial spreading, Neuroblastoma, esophageal squamous cell carcinoma, Osteosarcoma, Ovarian cancer, Prostate cancer, Renal clear cell carcinoma, Retinoblastoma, Rhabdomyosarcoma, and Small cell lung carcinoma;
the cancer associated with overexpression, translocation, amplification, or rearrangement of BET proteins is selected from NUT midline carcinoma, B-cell lymphoma, non-small cell lung cancer, esophageal cancer, head and neck squamous cell carcinoma, breast cancer, prostate cancer, and colon cancer;
the cancer that relies on pTEFb (Cdk9/cyclin T) and BET proteins to regulate oncogenes is selected from chronic lymphocytic leukemia, multiple myeloma, follicular lymphoma, diffuse large B-cell lymphoma, Burkitt's lymphoma, Hodgkin's lymphoma, anaplastic large cell lymphoma, neuroblastoma and primary neuroectodermal tumor, rhabdomyosarcoma, prostate cancer, and breast cancer;
the cancer associated with upregulation of BET responsive genes CDK6, Bcl2, TYRO3, MYB, and/or hTERT is selected from pancreatic cancer, breast cancer, colon cancer, glioblastoma, adenoid cystic carcinoma, T-cell prolymphocytic leukemia, malignant glioma, bladder cancer, medulloblastoma, thyroid cancer, melanoma, multiple myeloma, Barret's adenocarcinoma, hepatoma, prostate cancer, pro-myelocytic leukemia, chronic lymphocytic leukemia, mantle cell lymphoma, diffuse large B-cell lymphoma, small cell lung cancer, and renal carcinoma;
the cancer sensitive to effects of BET inhibition is selected from NUT-midline carcinoma (NMC), acute myeloid leukemia (AML), acute B lymphoblastic leukemia (B-ALL), Burkitt's Lymphoma, acute B-cell Lymphoma, Melanoma, mixed lineage leukemia, multiple myeloma, pro-myelocytic leukemia (PML), non-Hodgkin's lymphoma, Neuroblastoma, Medulloblastoma, lung carcinoma (NSCLC, SCLC), breast cancer, prostate cancer, and colon carcinoma; and
the cancer associated with a virus is associated with a virus selected from Epstein-Barr Virus (EBV), hepatitis B virus (HBV), hepatitis C virus (HCV), Kaposi's sarcoma associated virus (KSHV), human papilloma virus (HPV), Merkel cell polyomavirus, and human cytomegalovirus (CMV).

89. The method of claim 85, wherein the cancer is resistant to treatment with immunotherapy, hormone-deprivation therapy, and/or chemotherapy.

90. The method of claim 89, wherein administration of a therapeutically effective amount of said compound:
restores sensitivity to immunotherapy, hormone-deprivation therapy, and/or chemotherapy; and/or
inhibits proliferation of cancer cells, and/or induces cancer cell death or senescence.

91. The method of claim 85, wherein said compound of is combined with other therapies, chemotherapeutic agents, or antiproliferative agents.

92. A method of treating a benign proliferative or fibrotic disorder, selected from benign soft tissue tumors, bone tumors, brain and spinal tumors, eyelid and orbital tumors, granuloma, lipoma, meningioma, multiple endocrine neoplasia, nasal polyps, pituitary tumors, prolactinoma, pseudotumor cerebri, seborrheic keratoses, stomach polyps, thyroid nodules, cystic neoplasms of the pancreas, hemangiomas, vocal cord nodules, polyps, and cysts, Castleman disease, chronic pilonidal disease, dermatofibroma, pilar cyst, pyogenic granuloma, juvenile polyposis syndrome, idiopathic pulmonary fibrosis, renal fibrosis, post-operative stricture, keloid formation, scleroderma, and cardiac fibrosis comprising administering a therapeutically effective amount of the compound of claim 64.

93. A method of treating a disease or disorder selected from:
a disease or disorder that benefits from up-regulation of ApoA-I transcription and protein expression;
a metabolic disease or disorder; and
a neurological disease or disorder;
comprising administering a therapeutically effective amount of the compound of claim 64.

94. The method of claim 93, wherein:
the disease or disorder that benefits from up-regulation of ApoA-I transcription and protein expression is selected from cardiovascular disease, dyslipidemia, atherosclerosis, hypercholesterolemia, metabolic syndrome, and Alzheimer's disease;
the metabolic disease or disorder is selected from obesity-associated inflammation, type II diabetes, and insulin resistance; and
the neurological disease or disorder is selected from Alzheimer's disease, Parkinson's disease, Huntington disease, bipolar disorder, schizophrenia, Rubinstein-Taybi syndrome, and epilepsy.
